(19) United States
(12) Patent Application Publication
(10) Pub. No.: US 2024/0034760 A1
(43) Pub. Date: Feb. 1, 2024

(54) FUSION PROTEINS COMPRISING MILK PROTEINS AND COMPOSITIONS THEREOF

(71) Applicant: Perfect Day, Inc., Berkeley, CA (US)

(72) Inventors: Timothy Scott Johnson, Oakland, CA (US); Timothy Geistlinger, Oakland, CA (US); Ty B. Wagoner, Oakland, CA (US); Janine Tsan-Huei Lin, Davis, CA (US)

(73) Assignee: Perfect Day, Inc., Berkeley, CA (US)

(21) Appl. No.: 18/040,800

(22) PCT Filed: Aug. 5, 2021
(86) PCT No.: PCT/US21/44696

§ 371 Date: Feb. 6, 2023

Related U.S. Application Data
(60) Provisional application No. 63/061,656, filed on Aug. 5, 2020.

(51) Int. Cl. C07K 14/47; A61K 8/64; A61Q 19/00; C12P 21/02; C12N 15/80; A23L 33/19; A23J 1/20; A23J 1/18

(52) U.S. Cl. CPC C07K 14/4732; C07K 14/47; A61K 8/64; A61Q 19/00; C12P 21/02; C12N 15/80; A23L 33/19; A23J 1/202; A23J 1/18; C12N 2800/102

(57) ABSTRACT

The present invention relates generally to a recombinant protein, compositions comprising such recombinant protein, and to methods for producing such recombinant protein and compositions.

### RELATED APPLICATIONS

This application claims priority to U.S. Provisional Patent Application Ser. No. 63/061,656, filed on Aug. 5, 2020, which is incorporated herein by reference, in its entirety.

### FIELD OF THE INVENTION

The present invention relates generally to recombinant proteins, compositions comprising such recombinant proteins, and to methods for producing such recombinant proteins and compositions.

### BACKGROUND OF THE INVENTION

Petroleum-based polymers are used extensively in modern societies, for example, in industrial and consumer products. However, production, use, and disposal of petroleum-based polymers leave a large environmental footprint: the polymers are produced from monomers derived from non-renewable petroleum or natural gas sources, can leach harmful compounds, and are typically disposed of in landfill sites. Disposal via recycling has increased in recent years, but recycling rates remain low. Moreover, recycling of products that comprise different types of petroleum-based polymers and/or processing additives (e.g., fillers, coloring agents, plasticizers) is complex and not economical. The problems are not solved by newly developed photodegradable polymers, which are also made from petroleum-derived components, and which typically can be degraded by sunlight only to smaller particles of plastic rather than be decomposed completely.

Use of biopolymers for production of consumer goods dates back to ancient times. Biopolymers are derived from renewable materials (e.g., agricultural byproducts), are typically safe, and in many cases are fully biodegradable (i.e., degradable by bacteria, fungi, or other microorganisms). Traditional starting materials for producing biopolymers include milk proteins, starch, and cellulose.

Despite their favorable properties, milk proteins fell from favor as starting materials for biopolymers due to their expensive production via extraction from milk obtained from lactating mature animals. Moreover, such production yields fractions of mixed proteins (e.g., whey or casein protein isolates or protein concentrates comprising mixtures of whey proteins or caseins, respectively, as well as other components of milk (e.g., carbohydrates, minerals, lipids), making studying and predicting of behaviors in polymerization reactions and/or properties of resulting polymers difficult. Lastly, chemically produced monomers can be more easily modified in reaction vessels than milk proteins produced by whole organisms, adding to the versatility of petroleum-based polymers compared to biopolymers.

Such defects are overcome by recombinant production of milk proteins since biopolymers comprising recombinant milk proteins can better compete with petroleum-based polymers on cost of production and ability to predict and/or control polymerization reactions. Moreover, recombinant production of milk proteins provides opportunities for recombinant production of modified milk proteins, and production of biopolymers therefrom that have new attributes.

Therefore, a need exists for biopolymers made from recombinantly produced milk proteins.

### INCORPORATION BY REFERENCE

All publications, patents, patent applications, sequences, database entries, scientific publications, and other references mentioned herein are incorporated by reference in their entireties to the same extent as if each individual publication, patent, patent application, sequence, database entry, scientific publication, or other reference was specifically and individually indicated to be incorporated by reference. To the extent the material incorporated by reference contradicts or is inconsistent with the present disclosure, the present disclosure, including definitions, will supersede any such material.

### SUMMARY OF THE INVENTION

Paragraph 1: In various aspects, provided herein is a protein, wherein the protein is a recombinant protein having a structure:
(A) (milk protein 1)-(linker peptide 1)-(milk protein 2),
or
(B) (milk protein 1)-(linker peptide 1)-(milk protein 2)-(linker peptide 2)-(milk protein 3),
or
(C) (milk protein 1)-(linker peptide 1)-(milk protein 2)-(linker peptide 2)-(milk protein 3)-(linker peptide 3)-(milk protein 4);
wherein:
(i) the milk protein 1, the milk protein 2, the milk protein 3, the milk protein 4, the linker peptide 1, the linker peptide 2, and the linker peptide 3 are linked via peptide bonds; and
(ii) at least one of the milk protein 1, the milk protein 2, the milk protein 3, and the milk protein 4 is a milk protein comprising a free thiol group (e.g., a β-lactoglobulin comprising a free thiol group, a serum albumin comprising a free thiol group, or a lactoperoxidase comprising a free thiol group).

Paragraph 2: The recombinant protein of Paragraph 1 may have a structure (A), wherein the milk protein 1 is a milk protein comprising a free thiol group, and the milk protein 2 is a milk protein not comprising a free thiol group.

Paragraph 3: The recombinant protein of Paragraph 1 may have a structure (B), wherein the milk protein 1 is a milk protein comprising a free thiol group, and each of the milk protein 2 and the milk protein 3 is a milk protein not comprising a free thiol group.

Paragraph 4: The recombinant protein of Paragraph 1 may have a structure (B), wherein the milk protein 2 is a milk protein comprising a free thiol group, and each of the milk protein 1 and the milk protein 3 is a milk protein not comprising a free thiol group.

Paragraph 5: The recombinant protein of Paragraph 1 may have a structure (C), wherein the milk protein 1 is a milk protein comprising a free thiol group, and each of the milk protein 2, the milk protein 3, and the milk protein 4 is a milk protein not comprising a free thiol group.

Paragraph 6: The recombinant protein of Paragraph 1 may have a structure (C), wherein the milk protein 2 is a milk protein comprising a free thiol group, and each of the milk protein 1, the milk protein 3, and the milk protein 4 is a milk protein not comprising a free thiol group.

Paragraph 7: The recombinant protein of Paragraph 1 may have a structure (A), wherein each of the milk protein 1 and the milk protein 2 is a milk protein comprising a free thiol group.

Paragraph 8: The recombinant protein of Paragraph 1 may have a structure (B), wherein each of the milk protein 1 and the milk protein 2 is a milk protein comprising a free thiol group, and the milk protein 3 is a milk protein not comprising a free thiol group.

Paragraph 9: The recombinant protein of Paragraph 1 may have a structure (B), wherein each of the milk protein 1 and the milk protein 3 is a milk protein comprising a free thiol group, and the milk protein 2 is a milk protein not comprising a free thiol group.

Paragraph 10: The recombinant protein of Paragraph 1 may have a structure (C), wherein each of the milk protein 1 and the milk protein 2 is a milk protein comprising a free thiol group, and each of the milk protein 3 and the milk protein 4 is a milk protein not comprising a free thiol group.

Paragraph 11: The recombinant protein of Paragraph 1 may have a structure (C), wherein each of the milk protein 1 and the milk protein 3 is a milk protein comprising a free thiol group, and each of the milk protein 2 and the milk protein 4 is a milk protein not comprising a free thiol group.

Paragraph 12: The recombinant protein of Paragraph 1 may have a structure (C), wherein each of the milk protein 1 and the milk protein 4 is a milk protein comprising a free thiol group, and each of the milk protein 2 and the milk protein 3 is a milk protein not comprising a free thiol group.

Paragraph 13: The recombinant protein of Paragraph 1 may have a structure (C), wherein each of the milk protein 2 and the milk protein 3 is a milk protein comprising a free thiol group, and each of the milk protein 1 and the milk protein 4 is a milk protein not comprising a free thiol group.

Paragraph 14: The recombinant protein of Paragraph 1 may have a structure (B), wherein each of the milk protein 1, the milk protein 2, and the milk protein 3 is a milk protein comprising a free thiol group.

Paragraph 15: The recombinant protein of Paragraph 1 may have a structure (C), wherein each of the milk protein 1, the milk protein 2, and the milk protein 3 is a milk protein comprising a free thiol group, and the milk protein 4 is a milk protein not comprising a free thiol group.

Paragraph 16: The recombinant protein of Paragraph 1 may have a structure (C), wherein each of the milk protein 1, the milk protein 2, the milk protein 4 is a milk protein comprising a free thiol group, and the milk protein 3 is a milk protein not comprising a free thiol group.

Paragraph 17: The recombinant protein of Paragraph 1 may have a structure (C), wherein each of the milk protein 1, the milk protein 2, the milk protein 3, and the milk protein 4 is a milk protein comprising a free thiol group.

Paragraph 18: In various aspects, provided herein is a recombinant expression construct consisting of a polynucleotide comprising a promoter sequence, an optional secretion signal sequence, a polynucleotide sequence encoding a recombinant protein of any of Paragraphs 1 through 17, and a termination sequence; wherein the promoter sequence is operably linked in sense orientation to the optional secretion signal sequence and the polynucleotide sequence encoding the recombinant protein, the optional secretion signal sequence is operably linked in sense orientation to the polynucleotide sequence encoding the recombinant protein, and the one or more terminator sequences are operably linked to the polynucleotide sequence encoding the recombinant protein.

Paragraph 19: In various aspects, provided herein is a recombinant vector comprising the recombinant expression construct of Paragraph 18.

Paragraph 20: In various aspects, provided herein is a recombinant host cell comprising one or more (e.g., one, two, three, four, five, six, seven, eight, nine, ten, or more) of the recombinant expression construct of Paragraph 18. The recombinant host cell may be derived from a bacterium, fungus (e.g., yeast, filamentous fungus [e.g., a member of the genera Aspergillus, Trichoderma, Myceliophthora]), archaea, protista, animal, plant, algae, protozoan, or chromista.

Paragraph 21: In various aspects, provided herein is a method for obtaining the recombinant host cell of Paragraph 20, wherein the method comprises obtaining a polynucleotide encoding the recombinant protein, or obtaining the recombinant expression construct of Paragraph 18, or obtaining the recombinant vector of Paragraph 19; and introducing the polynucleotide, recombinant expression construct, or recombinant vector into a host cell.

Paragraph 22: In various aspects, provided herein is a method for producing the recombinant protein of any of Paragraphs 1 through 17, wherein the method comprises fermenting the recombinant host cell of Paragraph 20 in a culture medium under conditions suitable for production of the recombinant protein. The method may further comprise purifying the recombinant protein to obtain a preparation comprising the recombinant protein (e.g., a preparation having a purity of the recombinant protein of greater than 50% by mass of total protein) and/or post-processing the recombinant protein.

Paragraph 23: In various aspects, provided herein is a method for producing a casein (e.g., κ-casein, β-casein. α-S1-casein, α-S2-casein), wherein the method comprises: fermenting a recombinant host cell capable of producing a recombinant protein having a structure
(A) (milk protein 1)-(linker peptide 1)-(milk protein 2),
or
(B) (milk protein 1)-(linker peptide 1)-(milk protein 2)-(linker peptide 2)-(milk protein 3),
or
(C) (milk protein 1)-(linker peptide 1)-(milk protein 2)-(linker peptide 2)-(milk protein 3)-(linker peptide 3)-(milk protein 4);
wherein:
(i) the milk protein 1, the milk protein 2, the milk protein 3, the milk protein 4, the linker peptide 1, the linker peptide 2, and the linker peptide 3 are linked via peptide bonds,
(ii) at least one of the milk protein 1, the milk protein 2, the milk protein 3, and the milk protein 4 is a β-lactoglobulin,
(iii) at least one of the milk protein 1, the milk protein 2, the milk protein 3, and the milk protein 4 is the casein, and
(iv) wherein the casein is adjacent to one or more linker peptides comprising a recognition or cleavage sequence for a protease.

Paragraph 24: In the method of Paragraph 23 the recombinant protein may have a structure (A), wherein the milk protein 1 is a β-lactoglobulin, the milk protein 2 is the casein, and he linker peptide 1 comprises a recognition or cleavage sequence for a protease.

Paragraph 25: In the method of Paragraph 23 the recombinant protein may have a structure (B), wherein at least one of the milk protein 1 and the milk protein 2 is a β-lactoglobulin, the milk protein 2 is the casein, and the linker peptide 1 and the linker peptide 2 comprise a recognition or cleavage sequence for a protease.

Paragraph 26: In the method of Paragraph 23 the recombinant protein may have a structure (C), wherein at least one of milk protein 1 and milk protein 4 is a β-lactoglobulin; the milk protein 2 and the milk protein 3 is the casein; and the linker peptide 1, the linker peptide 2, and the linker peptide 3 comprise a recognition or cleavage sequence for a protease.

Paragraph 27: In the method of Paragraph 23 the recombinant protein may have a structure (C), wherein at least one of milk protein 1 and milk protein 3 is a β-lactoglobulin; the milk protein 2 and the milk protein 4 is the casein; and the linker peptide 1, the linker peptide 2, and the linker peptide 3 comprise a recognition or cleavage sequence for a protease.

Paragraph 28: In the method of Paragraph 23 the recombinant protein may have a structure (C), wherein the milk protein 1 is a β-lactoglobulin; the milk protein 2, the milk protein 3, and the milk protein 4 is the casein; and the linker peptide 1, the linker peptide 2, and the linker peptide 3 comprise a recognition or cleavage sequence for a protease (e.g., chymosin, trypsin, pepsin, matrix metalloproteinases, GI proteases, subtilisin, kex2 endoprotease, TEV protease).

Paragraph 29: The method of Paragraph 23 may further comprise cleaving the one or more linker peptides comprising the recognition or cleavage sequence for a protease with the protease, thereby releasing the casein.

Paragraph 30: In various aspects, provided herein is a polymer, wherein the polymer comprises a protein polymer, wherein the protein polymer comprises protein monomers linked via disulfide bonds, and wherein the protein monomers include one or more recombinant proteins (e.g., one recombinant protein, two or more recombinant proteins) of any of Paragraphs 1 through 17.

Paragraph 31: In various aspects, provided herein is a method for producing the polymer of Paragraph 30, wherein the method comprises: obtaining the one or more recombinant proteins of any of Paragraphs 1 through 17 (e.g., using the method of Paragraph 22), and mildly denaturing the one or more recombinant proteins (e.g., by heating the one or more recombinant proteins to a temperature [e.g., 10-30° C. below the Tm of the recombinant protein] at a pH at which the conformation of the one or more recombinant proteins is relatively stable or slightly unstable or unstable) such that one or more free thiol groups comprised in the one or more recombinant proteins form one or more intra- and/or inter-molecular disulfide bonds.

Paragraph 32: In various aspects, provided herein is a composition (e.g., a food product [e.g., a supplemented food product (e.g., supplemented dairy product, supplemented animal meat or animal meat product, supplemented egg or egg product), a substitute food product (e.g., substitute dairy product, substitute animal meat or animal meat product, substitute egg or egg product)], a cosmetic or personal care composition) comprising the recombinant protein of any of Paragraphs 1 through 17 and/or the polymer of any of Paragraph 30.

### BRIEF DESCRIPTION OF THE FIGURES

Example embodiments will be described and explained with additional specificity and detail through the use of the accompanying drawings in which:

FIG. 1 is a map of a targeting vector used for production of recombinant milk proteins provided herein, in accordance with representative embodiments of the present invention.

FIG. 2 is a plot that charts production of β-casein by recombinant fungal host cells comprising expression constructs encoding β-casein or encoding a recombinant protein having a structure of: (β-lactoglobulin)-(linker peptide 1)-(β-casein), in accordance with representative embodiments of the present invention.

### DETAILED DESCRIPTION OF THE INVENTION

The subsequent discussion of the invention is presented for purposes of illustration and description, and is not intended to limit the scope of the invention to the embodiments disclosed herein. As such, variations and modifications of the disclosed embodiments are within the scope of the invention, e.g., as may be within the skill and knowledge of those in the art after understanding the present disclosure. It is intended to obtain rights which include alternative embodiments to the extent permitted, including alternate, interchangeable and/or equivalent structures, functions, ranges or steps to those disclosed herein, and without intending to publicly dedicate any patentable subject matter. Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

The terms “a” and “an” and “the” and similar references as used herein refer to both the singular and the plural (e.g., meaning “at least one” or “one or more”), unless otherwise indicated herein or clearly contradicted by context. For example, the term “a compound” is synonymous with the terms “at least one compound” and “one or more compounds”, and may refer to a single compound or to a plurality of compounds, including mixtures thereof.

The term “and/or” as used herein refers to multiple components in combination with or exclusive of one another. For example, “x, y, and/or z” may refer to “x” alone, “y” alone, “z” alone, “x, y, and z”, “(x and y) or z”, “(x and z) or y”, “(y and z) or x”, “x and y” alone, “x and z” alone, “y and z” alone, or “x or y or z”.

The term “at least” or “one or more” as used herein refers to one, two, three, four, five, six, seven, eight, nine, ten, at least one, at least two, at least three, at least four, at least five, at least six, at least seven, at least eight, at least nine, at least ten, or more, or all of the elements subsequently listed.

The term “casein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a casein natively found in a mammal-produced milk (i.e., a casein that is native to a mammal-produced milk; e.g., a native casein). Examples of caseins include β-casein, κ-casein, α-S1-casein, and α-S2-casein. Accordingly, the terms “β-casein”, “κ-casein”, “α-S1-casein”, and “α-S2-casein” as used herein refer to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a β-casein, κ-casein, α-S1-casein, and α-S2-casein, respectively, natively found in a mammal-produced milk (e.g., Bos taurus β-casein (amino acids 16 to 224 of UniProt sequence P02666), Bos taurus κ-casein (amino acids 22 to 190 of UniProt sequence P02668), Bos taurus α-S1-casein (amino acids 16 to 214 of UniProt sequence P02662), and Bos taurus α-S2-casein (amino acids 16 to 222 of UniProt sequence P02663), respectively). Differences between the amino acid sequences of a casein and that of a casein natively found in a mammal-produced milk may be due to conservative amino acid substitutions (i.e., replacement of amino acids with chemically similar amino acids; conservative substitution tables providing functionally similar amino acids are well known in the art), non-conservative amino acid substitutions, and amino acid insertions, amino acid deletions, and polypeptide truncations (e.g., providing a fragment of a casein) that do not materially alter structure and/or one or more functions of the casein. Such amino acid substitutions/deletions/insertions may be identified using methods known in the art, which involve producing a casein comprising a candidate amino acid substitution/deletion/insertion identified through molecular modeling approaches (using, for example, PyMol [Schrödinger, New York, NY]) or multi-sequence alignments (e.g., of orthologs of native caseins; using, for example, MUSCLE [Edgar, 2004, Nucleic Acids Res 32: 1792-1797]) as not materially altering protein structure and/or function, and testing such casein using well-known methods for determining protein structure and/or function. Such methods may also be employed to determine whether a casein is useful in the present invention, and to identify a casein that has an improved function in a specific applications.

The term “encoding” as used herein in context of a polynucleotide refers to a polynucleotide that comprises coding a sequence that when placed under the control of appropriate regulatory sequences is transcribed into mRNA that can be translated into a polypeptide. A coding sequence generally starts at a start codon (e.g., ATG) and ends at a stop codon (e.g., UAA, UAG and UGA). A coding sequence may contain a single open reading frame, or several open reading frames (e.g., separated by introns).

The term “essentially free of” as used herein refers to the indicated component being either not detectable in the indicated composition by common analytical methods, or to the indicated component being present in such trace amount as to not be functional. The term “functional” as used in this context refers to not materially contributing to properties of the composition comprising the trace amount of the indicated component, or to not having material activity (e.g., chemical activity, enzymatic activity) in the indicated composition comprising the trace amount of the indicated component, or to not having health-adverse effects upon use or consumption of the composition comprising the trace amount of the indicated component. The term “materially contributing” as used herein refers to the indicated component contributing to an attribute of a composition to such extent that in the absence of the component (e.g., in a reference composition that is identical to the composition except that it lacks the indicated component) the attribute is at least 10%, at least 20%, at least 30%, at least 40%, or at least 50% less present/active/measurable.

The term “filamentous fungus” as used herein refers to an organism from the filamentous form of the subdivision Eumycota and Oomycota (as defined by Hawksworth et al., In, Ainsworth and Bisby's Dictionary of The Fungi, 8th edition, 1995, CAB International, University Press, Cambridge, UK). A filamentous fungus is distinguished from a yeast by its hyphal elongation during vegetative growth.

The term “food product” as used herein refers to a composition that can be ingested by a human or an animal for dietary purposes (i.e., without ill health effects but with significant nutritional and/or caloric intake due to uptake of digested material in the gastrointestinal tract), including a domesticated animal (e.g., dog, cat), farm animal (e.g., cow, pig, horse), and wild animal (e.g., non-domesticated predatory animal). The term includes compositions that may be combined with or added to one or more other ingredients to make a food product that can be ingested by a human or an animal.

The term “free thiol group” as used herein refers to the thiol-containing sidechain of a cysteine residue in which the hydrogen atom in the thiol group is not replaced by another atom. For example, a milk protein that comprises an odd number of cysteine residues may both in reducing and in non-reducing conditions comprise a free thiol group.

The term “fungus” as used herein refers to organisms of the phyla Ascomycotas, Basidiomycota, Zygomycota, and Chythridiomycota, Oomycota, and Glomeromycota. It is understood, however, that fungal taxonomy is continually evolving, and therefore this specific definition of the fungal kingdom may be adjusted in the future. The term “fungal host cell” as used herein refers to a host cell that is obtained from a fungus.

The term “homolog” as used herein refers to a protein that comprises an amino acid sequence that is at least 40% (e.g., at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100%) identical to a sequence of amino acids of a similar length (i.e., a length that is within +/−20% of the length of the query amino acid sequence) comprised in a reference protein, and that has a functional property that is similar to (e.g., is within 50%, within 40%, within 30%, within 20%, or within 10% of) that of the reference protein. The term includes polymorphic variants, interspecies homologs (e.g., orthologs), paralogs, and alleles of a protein.

The term “host cell” as used herein refers not only to the particular subject cell but to the progeny of such cell. Because certain modifications may occur in succeeding generations due to either mutation or environmental influences, such progeny may not, in fact, be identical to the subject cell, but are still included within the scope of the term “host cell” as used herein.

The terms “identity” or “identical” in the context of two or more polynucleotides or polypeptides as used herein refer to the nucleotide or amino acid residues that are the same when the two or more polynucleotide or polypeptides, respectively, are aligned for maximum correspondence. Depending on the application, the “identity” may exist over a region of the sequences being compared (e.g., over the length of a functional domain) or over the full length of the sequences. A “region” is considered to be a continuous stretch of at least 9, 14, 19, 24, 29, 34, 39, or more nucleotides, or of at least 6, 10, 14, 18, 22, 26, 30, or more amino acids. For comparison, typically one sequence acts as a reference sequence to which one or more test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison may be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Sequence Analysis Software Package of the Genetics Computer Group (GCG), University of Wisconsin Biotechnology Center, which may be used with default parameters), or by visual inspection (see generally Ausubel et al., infra). One example of an algorithm that is suitable for determining percent sequence identity and sequence similarity is the BLAST algorithm (see, for example, Altschul et al. [1990] J. Mol. Biol. 215:403-410; Gish & States. [1993] Nature Genet. 3:266-272; Madden et al. [1996] Meth. Enzymol. 266:131-141; Altschul et al. [1997] Nucleic Acids Res. 25:3389-3402; Zhang 7 Madden. [1997] Genome Res. 7:649-656). Software for performing BLAST analyses is publicly available through the National Center for Biotechnology Information.

The terms “including,” “includes,” “having,” “has,” “with,” or variants thereof as used herein are intended to be inclusive in a manner similar to the term “comprising”.

The term “linker peptide” as used herein refers to a sequence of amino acids that is positioned between separate domains (i.e., milk proteins) of the recombinant protein provided herein. The linker peptide may include from 1 to 80 amino acids.

The term “mammal-produced milk” as used herein refers to a milk produced by a mammal.

The term “milk protein” as used herein refers to a whey protein or a casein. The milk protein may be derived from any mammalian species, including but not limited to cow, human, sheep, mouflon, goat, buffalo, camel, horse, donkey, alpaca, yak, llama, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, and echidna.

The term “native” as used herein refers to what is found in nature in its unmodified state (e.g., a cell that is not genetically modified by a human, and that is maintained under conditions [e.g., level of oxygenation, pH, salt concentration, temperature, and nutrient (e.g., carbon, nitrogen, sulfur) availability] that are not defined by a human).

The term “operably linked” as used herein refers to an arrangement of elements that allows them to be functionally related. For example, a promoter sequence is operably linked to a protein coding sequence if it controls the transcription of the protein coding sequence, and a secretion signal sequence is operably linked to a protein if the secretion signal sequence directs the protein through the secretion system of a cell. An “operably linked” element may be in contiguous linkage with another element, or act in trans or at a distance to another element. Non-limiting examples of functions that may be operably linked include control of transcription, control of translation, protein folding, and protein secretion.

The term “one or more” as used herein refers to one, at least one, two, three, four, five, six, seven, eight, nine, ten, or more, or all of the elements subsequently listed.

The terms “optional” or “optionally” as used herein refer to a feature or structure being present or not, or an event or circumstance occurring or not. The description includes instances in which a feature or structure is present, instances in which a feature or structure is absent, instances in which an event or circumstance occurs, and instances in which an event or circumstance does not occur.

The term “peptide bond” as used herein refers to a eupeptide bond (i.e., an amide type of covalent chemical bond linking two consecutive alpha-amino acids from C1 of one alpha-amino acid and N2 of another, along a polypeptide or protein chain).

The term “polymer” refers to a polymeric compound prepared by polymerizing protein monomers, whether of the same or a different type. The generic term polymer thus embraces the term “homopolymer,” usually employed to refer to polymers prepared from only one protein monomer, as well as “copolymer”, which refers to polymers prepared from two or more different monomers. The “polymer” may be a straight chain with or without branches, and may include a polymer matrix (e.g., interconnected polymer strands).

The term “polynucleotide” as used herein refers to a polymeric form of at least 2 (e.g., at least 5, at least 10, at least 20, at least 30, at least 40, at least 50, at least 100, at least 500, at least 1,000) nucleotides. The term includes both sense and antisense strands of DNA molecules (e.g., cDNA, genomic DNA, synthetic DNA) and RNA molecules (e.g., mRNA, synthetic RNA), as well as analogs of DNA or RNA containing non-natural nucleotide analogs, non-native internucleoside bonds, and/or chemical modifications. A polynucleotide may be modified chemically or biochemically or may contain non-natural or derivatized nucleotide bases. Such modifications include, for example, labels; methylation; substitution of one or more of the naturally occurring nucleotides with an analog; internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Examples of modified nucleotides are described in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding or other chemical interaction. Such molecules are known in the art and include, for example, molecules in which peptide linkages substitute for phosphate linkages in the backbone of the molecule. Other modifications may include, for example, analogs in which the ribose ring contains a bridging moiety or other structure such as the modifications found in “locked” polynucleotides. A polynucleotide may be in any topological conformation. For instance, a polynucleotide may be single-stranded, double-stranded, triple-stranded, quadruplexed, partially double-stranded, branched, hairpinned, circular, or in a padlocked conformation. The term “polynucleotide sequence” as used herein refers to a sequence of nucleotides that are comprised in a polynucleotide or of which a polynucleotide consists.

The terms “polypeptide” and “protein” as used herein can be interchanged, and refer to a naturally-occurring or a naturally not occurring polymeric form of at least 2 (e.g., at least 5, at least 10, at least 20, at least 30, at least 40, at least 50, at least 100) amino acids. A “polypeptide” or “protein” may have an active structure or lacking a functional structure, comprise coded and/or non-coded amino acids, comprise amino acids that occur in nature and/or amino acids that do not occur in nature, comprise chemically modified and/or biochemically modified and/or derivatized amino acids, comprise unmodified and/or modified peptide backbones, and/or be monomeric (i.e., having a single chain) or polymeric (i.e., having of two or more chains, which may be covalently or non-covalently associated). The term “amino acid sequence” as used herein refers to a sequence of amino acids that is comprised in a “polypeptide” or “protein”, or of which a “polypeptide” or “protein” consists.

The term “post-translational modification”, or its acronym “PTM”, as used herein refers to the covalent attachment of a chemical group to a polypeptide after biosynthesis. PTM may occur on an amino acid side chain of the polypeptide or at its C- or N-termini. Non-limiting examples of PTMs include glycosylation (i.e., covalent attachment to proteins of glycan groups (e.g., monosaccharides, disaccharides, polysaccharides, linear glycans, branched glycans, glycans with galf residues, glycans with sulfate and/or phosphate residues, D-glucose, D-galactose, D-mannose, L-fucose, N-acetyl-D-galactose amine, N-acetyl-D-glucose amine, N-acetyl-D-neuraminic acid, galactofuranose, phosphodiesters, N-acetylglucosamine, N-acetylgalactosamine, sialic acid, and combinations thereof; see, for example, Deshpande et al. 2008. Glycobiology 18(8):626) via C-linkage (i.e., C-glycosylation), N-linkage (i.e., N-glycosylation), or O-linkage (i.e., O-glycosylation), or via glypiation (i.e., addition of a glycosylphosphatidylinositol anchor) or phosphoglycosylation (i.e., linked through the phosphate of a phospho-serine)), phosphorylation (i.e., covalent attachment to proteins of phosphate groups), alkylation (i.e., covalent attachment to proteins of alkane groups (e.g., methane group in methylation)), lipidation (i.e., covalent attachment of a lipid group (e.g., isoprenoid group in prenylation and isoprenylation (e.g., farnesol group in farnesylation, geraniol group in geranylation, geranylgeraniol group in geranylgeranylation), fatty acid group in fatty acylation (e.g., myristic acid in myristoylation, palmitic acid in palmitoylation), glycosylphosphatidylinositol anchor in glypiation)), hydroxylation (i.e., covalent attachment of a hydroxide group), sumoylation (i.e., attachment to proteins of Small Ubiquitin-like Modifier (or SUMO) protein), nitrosylation (i.e., attachment to proteins of an NO group; e.g., S-nitrosylation), nitrosothiolation (i.e., attachment to a cysteine thiol in a protein of an NO group (e.g., S-nitrosothiol)), glutathionylation (i.e., attachment to a cysteine thiol in a protein of a glutathione group (e.g., S-glutathionylation)), and tyrosine nitration (i.e., attachment to tyrosine residues of proteins of nitrate groups).

The term “promoter sequence” as used herein refers to a polynucleotide that directs transcription of a downstream polynucleotide in a cell. A promoter sequence may include necessary nucleotides near the start site of transcription, such as, in the case of a polymerase II type promoter, a TATA element. A promoter sequence may also optionally include distal enhancer or repressor elements, which may be located as much as several thousand base pairs from the start site of transcription.

The term “protease” as used herein refers to a protein that can hydrolyze (i.e., cleave) a peptide bond (e.g., members of enzyme classification groups EC 3.4). Non-limiting examples of proteases include chymosin, trypsin, pepsin, matrix metalloproteinases, GI proteases (e.g., proteases that are present in a human or other animal gastrointestinal tract and that are of human or microbial origin), subtilisin (UniProt #P00782), kex2 endoprotease (UniProt #P13134), and TEV protease (UniProt #P04517).

The term “protease recognition or cleavage sequence” or “recognition or cleavage sequence for a protease” as used herein refers to an amino acid sequence that is recognized by a protease and in which a peptide bond is cleaved by the protease.

The term “protein concentrate” as used herein refers to a protein material that is obtained upon removal of at least a portion (or a substantial portion) of one or more of carbohydrates, lipids, ash, and other minor constituents. It typically comprises between about 30% and about 80% (e.g., between 30% and 80%, 75%, 70%, 65%, 60%, 55%, 50%, 45%, 40%, or 35%; between 35% and 80%, 75%, 70%, 65%, 60%, 55%, 50%, 45%, or 40%; between 40% and 80%, 75%, 70%, 65%, 60%, 55%, 50%, or 45%; between 45% and 80%, 75%, 70%, 65%, 60%, 55%, or 50%; between 50% and 80%, 75%, 70%, 65%, 60%, or 55%; between 55% and 80%, 75%, 70%, 65%, or 60%; between 60% and 80%, 75%, 70%, or 65%; between 65% and 80%, 75%, or 70%; between 70% and 80%, or 75%; or between 75% and 80%) by mass of protein.

The term “protein isolate” as used herein refers to a protein material that is obtained upon removal of at least a portion (or a substantial portion) of one or more of polysaccharides, soluble carbohydrates, ash, and other minor constituents. It typically comprises at least 80% (i.e., at least 80%, at least 85%, at least 95%, at least 99%) by mass of protein.

The term “purifying” or “purified” or “isolating” or “isolated” as used herein refers to a component being substantially separated from chemicals, cellular components, and cells (e.g., cell walls, membrane lipids, chromosomes, other proteins, other cells in an organism) of the source from which the component originated. The component may be at least 60% pure, e.g., greater than 65%, 70%, 75%, 80%, 85%, 90%, 95%, or 99% pure. The term does not require (albeit allows) that the component be separated from all chemicals, cellular components, and cells.

The term “recombinant” as used herein in reference to a protein (e.g., a milk protein) refers to a protein that is produced in a recombinant host cell, or to a protein that is synthesized from a recombinant polynucleotide.

The term “recombinant host cell” as used herein refers to a host cell that comprises a recombinant polynucleotide. Thus, for example, a recombinant host cell may produce a polynucleotide or polypeptide not found in the native (non-recombinant) form of the host cell, or a recombinant host cell may produce a polynucleotide or polypeptide at a level that is different from that in the native (non-recombinant) form of the host cell. It should be understood that such term is intended to refer not only to the particular subject cell but also to the progeny of such a cell. Because certain modifications may occur in succeeding generations due to either mutation or environmental influences, such progeny may not be identical to the subject cell, but are still included within the scope of the term “recombinant host cell” as used herein. A recombinant host cell may be an isolated cell or cell line grown in culture, or may be a cell which resides in a living tissue or organism.

The term “recombinant polynucleotide” as used herein refers to a polynucleotide that is removed from its naturally occurring environment, or a polynucleotide that is not associated with all or a portion of a polynucleotide abutting or proximal to the polynucleotide when it is found in nature, or a polynucleotide that is operatively linked to a polynucleotide that it is not linked to in nature, or a polynucleotide that does not occur in nature, or a polynucleotide that contains a modification that is not found in that polynucleotide in nature (e.g., insertion, deletion, or point mutation introduced artificially, e.g., by human intervention), or a polynucleotide that is integrated into a chromosome at a heterologous site. The term can be used, e.g., to describe cloned DNA isolates, or a polynucleotide comprising a chemically synthesized nucleotide analog. A polynucleotide is also considered “recombinant” if it contains a genetic modification that does not naturally occur. For instance, an endogenous polynucleotide is considered a “recombinant polynucleotide” if it contains an insertion, deletion, or substitution of one or more nucleotides that is introduced artificially (e.g., by human intervention). Such modification may introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a host cell's chromosome, as well as a polynucleotide that is not in a host cell's chromosome (e.g., a polynucleotide that is comprised in an episome). A recombinant polynucleotide in a host cell or organism may replicate using the in vivo cellular machinery of the host cell; however, such recombinant polynucleotide, although subsequently replicated intracellularly, is still considered recombinant for purposes of this invention.

The term “regulatory element” as used herein refers a polynucleotide sequence that mediates, modulates, or regulates expression (e.g., transcription, post-transcriptional events, translation) of a polynucleotide to which the regulatory element is operably linked.

The term “secretion signal” as used herein refers to a peptide that is operably linked to the N-terminus of a protein, and that mediates the delivery of the protein via the intracellular secretory pathway of a host cell in which the protein is produced (i.e., synthesized) to the exterior of the host cell. Typically, operable linkage of a recombinant protein with a secretion signal requires removal of a start codon of the polynucleotide sequence encoding the recombinant protein.

The term “similar” us used herein refers to being within about +/−15% with regard to a specified attribute. The term includes being within +/−9%, +/−8%, +/−7%, +/−6%, +/−5%, +/−4%, +/−3%, +/−2%, or +/−1% with regard to the specified attribute.

The term “two or more” as used herein refers to two, at least two, three, four, five, six, seven, eight, nine, ten, or more, or all of the elements subsequently listed.

The term “whey protein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a whey protein natively found in a mammal-produced milk (i.e., a whey protein that is native to a mammal-produced milk; e.g., a native whey protein). Examples of whey proteins include α-lactalbumin, β-lactoglobulin, lactotransferrin, lactoferricin, serum albumin protein, lactoperoxidase protein, and glycomacropeptide. Accordingly, the terms “α-lactalbumin”, “β-lactoglobulin”, “lactotransferrin”, “lactoferricin”, “serum albumin”, “lactoperoxidase”, and “glycomacropeptide” as used herein refer to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in an α-lactalbumin, β-lactoglobulin, lactotransferrin, lactoferricin, serum albumin, lactoperoxidase, and glycomacropeptide (GMP), respectively, natively found in a mammal-produced milk (e.g., Bos taurus α-lactalbumin (amino acids 20-142 of UniProt sequence P00711), Bos taurus β-lactoglobulin (amino acids 17-178 of UniProt sequence P02754), Bos taurus lactotransferrin (amino acids 20 to 708 of UniProt sequence P24627), Bos taurus lactoferricin (amino acids 36 to 60 of UniProt sequence P24627), Bos taurus serum albumin (amino acids 25 to 607 of UniProt sequence P02769), Bos taurus lactoperoxidase (amino acids 101 to 712 of UniProt sequence P80025), and Bos taurus glycomacropeptide (GMP; amino acids 127 to 190 of UniProt sequence P02668), respectively). Differences between the amino acid sequences of a whey protein and that of a whey protein natively found in a mammal-produced milk may be due to conservative amino acid substitutions (i.e., replacement of amino acids with chemically similar amino acids; conservative substitution tables providing functionally similar amino acids are well known in the art), non-conservative amino acid substitutions, amino acid insertions, amino acid deletions, and polypeptide truncations (e.g., providing a fragment of a whey protein) that do not materially alter structure and/or one or more functions of the whey protein. Such amino acid substitutions/deletions/insertions may be identified using methods known in the art, which involve producing a whey protein comprising a candidate amino acid substitution/deletion/insertion identified through molecular modeling approaches (using, for example, PyMol [Schrödinger, New York, NY]) or multi-sequence alignments (e.g., of orthologs of native whey proteins; using, for example, MUSCLE [Edgar, 2004, Nucleic Acids Res 32: 1792-1797]) as not materially altering protein structure and/or function, and testing such whey protein using well-known methods for determining protein structure and/or function. Such methods may also be employed to determine whether a whey protein is useful in the present invention, and to identify a whey protein that has an improved function in a specific applications.

The term “vector” as used herein refers to a nuclei acid that can carry a polynucleotide sequence to be introduced into a host cell. Non-limiting examples of vectors include cloning vectors, expression vectors, shuttle vectors, plasmids, phage particles, viral vectors, cosmids, bacterial artificial chromosomes (BACs), yeast artificial chromosomes (YACs), virus particles (e.g., comprising heterologous polynucleotides), DNA constructs (e.g., produced by cloning or PCR amplification), and linear double-stranded molecules (e.g., PCR fragments). Certain vectors are capable of autonomous replication in a host cell into which they are introduced (e.g., vectors having an origin of replication which functions in the host cell). Other vectors may be integrated into the genome of a host cell upon introduction into the host cell, and are thereby replicated along with the host genome.

The term “yeast” as used herein refers to any organism of the order Saccharomycetales. Vegetative growth of yeast is by budding/blebbing of a unicellular thallus, and carbon catabolism may be fermentative.

The term “% by mass” as used herein refers to a percentage value for a mass as determined in a hydrated composition, such that the composition includes the mass of powder as well as the mass of the hydrating agent, with 100% fixed as the percentage value for the entire hydrated composition. In embodiments in which the composition is in powder form (to which the mass of the hydrating agent will be added at a later time), the term refers to a percentage value for a mass as determined relative to the eventual entire hydrated composition (with 100% fixed as the percentage value for that entire eventual hydrated composition).

Recitation of ranges of values herein are merely intended to serve as a shorthand method of referring individually to each separate value (fractional or integral) falling within the range inclusive of the recited minimum and maximum value, unless otherwise indicated herein, and each separate value is incorporated into the specification as if it were individually recited herein. Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of less than or equal to 10. It should further be understood that all ranges and quantities described below are approximations and are not intended to limit the invention.

It should be understood that in any method disclosed herein the order of steps or order for performing certain actions is immaterial so long as the invention remains operable. Moreover, two or more steps or actions may be conducted simultaneously.

It should be further understood that any structure disclosed herein for a recombinant protein may be read from C-terminus to N-terminus of the recombinant protein, or may be read from N-terminus to C-terminus of the recombinant protein. For example, disclosure of a recombinant protein having a structure (β-lactoglobulin)-(linker peptide 1)-(casein) may be read as a recombinant protein having β-lactoglobulin at its C-terminus and casein at its N-terminus, or may be read as having casein at its C-terminus and β-lactoglobulin at its N-terminus.

### Recombinant Protein

In various aspects, provided herein is a recombinant protein having a structure:
(A) (milk protein 1)-(linker peptide 1)-(milk protein 2),
or
(B) (milk protein 1)-(linker peptide 1)-(milk protein 2)-(linker peptide 2)-(milk protein 3),
or
(C) (milk protein 1)-(linker peptide 1)-(milk protein 2)-(linker peptide 2)-(milk protein 3)-(linker peptide 3)-(milk protein 4);
wherein:
   the milk protein 1, the milk protein 2, the milk protein 3, the milk protein 4, the linker peptide 1, the linker peptide 2, and the linker peptide 3 are linked via peptide bonds; and
   at least one of the milk protein 1, the milk protein 2, the milk protein 3, and the milk protein 4 is a milk protein comprising a free thiol group.

In the recombinant protein according to any of the above, the milk protein 1, the milk protein 2, the milk protein 3, and/or the milk protein 4 may be identical to each other or differ from each other in length and/or amino acid sequence; and/or the linker peptide 1, the linker peptide 2, and/or the linker peptide 3 may be identical to each other or differ from each other in length and/or amino acid sequence.

Milk proteins comprising a free thiol group include certain β-lactoglobulins (e.g., Bos taurus β-lactoglobulin [amino acids 17-178 of UniProt sequence P02754) other than those noted below wherein a thiol group is removed from the protein, certain serum albumins (e.g., Bos taurus serum albumin protein [amino acids 25 to 607 of UniProt sequence P02769]) other than those noted below wherein a thiol group is removed from the protein, and certain lactoperoxidases (e.g., Bos taurus lactoperoxidase [amino acids 101 to 712 of UniProt sequence P80025]) other than those noted below wherein a thiol group is removed from the protein. Milk proteins not comprising a free thiol group include certain α-lactalbumins (e.g., Bos taurus α-lactalbumin [amino acids 20-142 of UniProt sequence P00711]), certain lactotransferrins (e.g., Bos taurus lactotransferrin [amino acids 20 to 708 of UniProt sequence P24627]), certain lactoferricins (e.g., Bos taurus lactoferricin [amino acids 36 to 60 of UniProt sequence P24627]), certain glycomacropeptides (GMP; e.g., Bos taurus GMP [amino acids 127 to 190 of UniProt sequence P02668]), certain β-caseins (e.g., Bos taurus β-casein [amino acids 16 to 224 of UniProt sequence P02666]), certain κ-caseins (e.g., Bos taurus κ-casein [amino acids 22 to 190 of UniProt sequence P02668]), certain α-S1-caseins (e.g., Bos taurus α-S1-casein [amino acids 16 to 214 of UniProt sequence P02662]), certain α-S2-caseins (e.g., Bos taurus α-S2-casein [amino acids 16 to 222 of UniProt sequence P02663]), certain β-lactoglobulins (e.g., Bos taurus β-lactoglobulin [amino acids 17-178 of UniProt sequence P02754] comprising an amino acid substitution at position 137 from cysteine to serine whereby a thiol group is removed from the protein), certain serum albumins (e.g., Bos taurus serum albumin protein [amino acids 25 to 607 of UniProt sequence P02769] comprising an amino acid substitution at position 58 from cysteine to serine whereby a thiol group is removed from the protein), and certain lactoperoxidases (e.g., Bos taurus lactoperoxidase [amino acids 101 to 712 of UniProt sequence P80025] comprising an amino acid substitution at position 558 from cysteine to serine whereby a thiol group is removed from the protein). Those of skill in the art can readily ascertain whether a milk protein comprises a free thiol group or does not comprise a free thiol group (e.g., by determining whether a milk protein comprises an odd number of cysteine residues, and/or by analyzing the structure of a milk protein with regard to the presence of free, non-disulfide bonded thiol groups using methods known in the art, such as, for example, mass spectrometry (MS) analysis following proteolytic digestion of a protein under partial reducing or non-reducing conditions, and analysis of protein crystal structures).

Without wishing to be bound by theory, a recombinant protein according to any of the above in non-reducing conditions (e.g., conditions in which existing intramolecular disulfide bonds remain unchanged) can, through the available free thiol group(s), form a covalent linkage (i.e., a disulfide bridge) to another recombinant protein according to any of the above, thereby forming a variety of polymers, which cannot be formed or cannot be formed to the same extent or to the same purity using the milk protein 1, the milk protein 2, the milk protein 3, and/or the milk protein 4 in unlinked (i.e., not covalently linked to each other via peptide bonds) form, and that may contribute to a composition comprising or consisting essentially of the recombinant protein, and/or polymer formed from the recombinant protein, having various attributes.

### Milk Protein 1, Milk Protein 2, Milk Protein 3, Milk Protein 4

In a recombinant protein having a structure (A), (B), or (C) according to the above, only one of the milk protein 1, the milk protein 2, the milk protein 3, or the milk protein 4 may be a milk protein comprising a free thiol group (i.e., the recombinant protein comprises a monovalent free thiol group).

Accordingly, the recombinant protein may have:
a structure (A) according to the above wherein the milk protein 1 is a milk protein comprising a free thiol group and the milk protein 2 is a milk protein not comprising a free thiol group;
a structure (B) according to the above wherein the milk protein 1 is a milk protein comprising a free thiol group, the milk protein 2 is a milk protein not comprising a free thiol group, and the milk protein 3 is a milk protein not comprising a free thiol group;
a structure (B) according to the above wherein the milk protein 1 is a milk protein not comprising a free thiol group, the milk protein 2 is a milk protein comprising a free thiol group, and the milk protein 3 is a milk protein not comprising a free thiol group;
a structure (C) according to the above wherein the milk protein 1 is a milk protein comprising a free thiol group, the milk protein 2 is a milk protein not comprising a free thiol group, the milk protein 3 is a milk protein not comprising a free thiol group, and the milk protein 4 is a milk protein not comprising a free thiol group; or
a structure (C) according to the above wherein the milk protein 1 is a milk protein not comprising a free thiol group, the milk protein 2 is a milk protein comprising a free thiol group, the milk protein 3 is a milk protein not comprising a free thiol group, and the milk protein 4 is a milk protein not comprising a free thiol group.

Non-limiting examples of such recombinant proteins include:
a structure (A) according to the above having the form: (β-lactoglobulin)-(linker peptide 1)-(α-lactalbumin);
a structure (A) according to the above having the form: (bovine serum albumin)-(linker peptide 1)-(α-lactalbumin);
a structure (A) according to the above having the form: (lactoperoxidase)-(linker peptide 1) -(α-lactalbumin);
a structure (B) according to the above having the form: (β-lactoglobulin)-(linker peptide 1) -(α-lactalbumin)-(linker peptide 1)-(α-lactalbumin);
a structure (B) according to the above having the form: (bovine serum albumin)-(linker peptide 1)-(α-lactalbumin)-(linker peptide 1)-(α-lactalbumin);
a structure (B) according to the above having the form: (lactoperoxidase)-(linker peptide 1) -(α-lactalbumin)-(linker peptide 1)-(α-lactalbumin);
a structure (B) according to the above having the form: (α-lactalbumin)-(linker peptide 1) -(β-lactoglobulin)-(linker peptide 2)-(α-lactalbumin);
a structure (B) according to the above having the form: (α-lactalbumin)-(linker peptide 1)-(bovine serum albumin)-(linker peptide 2)-(α-lactalbumin);
a structure (B) according to the above having the form: (α-lactalbumin)-(linker peptide 1)-(lactoperoxidase)-(linker peptide 2)-(α-lactalbumin);
a structure (C) according to the above having the form: (β-lactoglobulin)-(linker peptide 1) -(α-lactalbumin)-(linker peptide 2)-(α-lactalbumin)-(linker peptide 3)-(α-lactalbumin);
a structure (C) according to the above having the form: (bovine serum albumin)-(linker peptide 1)-(α-lactalbumin)-(linker peptide 2)-(α-lactalbumin)-(linker peptide 3) -(α-lactalbumin); and
a structure (C) according to the above having the form: (lactoperoxidase)-(linker peptide 1) -(α-lactalbumin)-(linker peptide 2)-(α-lactalbumin)-(linker peptide 3)-(α-lactalbumin);
a structure (C) according to the above having the form: (α-lactalbumin)-(linker peptide 1) -(β-lactoglobulin)-(linker peptide 2)-(α-lactalbumin)-(linker peptide 3)-(α-lactalbumin);
a structure (C) according to the above having the form: (α-lactalbumin)-(linker peptide 1)-(bovine serum albumin)-(linker peptide 2)-(α-lactalbumin)-(linker peptide 3) -(α-lactalbumin); and
a structure (C) according to the above having the form: (α-lactalbumin)-(linker peptide 1)-(lactoperoxidase)-(linker peptide 2)-(α-lactalbumin)-(linker peptide 3)-(α-lactalbumin).

In a recombinant protein having a structure (A), (B), or (C) according to the above, only two of the milk protein 1, the milk protein 2, the milk protein 3, and the milk protein 4 may be a milk protein comprising a free thiol group (i.e., the recombinant protein comprises divalent free thiol groups).

Accordingly, the recombinant protein may have:
a structure (A) according to the above wherein the milk protein 1 is a milk protein comprising a free thiol group and the milk protein 2 is a milk protein comprising a free thiol group;
a structure (B) according to the above wherein the milk protein 1 is a milk protein comprising a free thiol group, the milk protein 2 is a milk protein comprising a free thiol group, and the milk protein 3 is a milk protein not comprising a free thiol group;
a structure (B) according to the above wherein the milk protein 1 is a milk protein comprising a free thiol group, the milk protein 2 is a milk protein not comprising a free thiol group, and the milk protein 3 is a milk protein comprising a free thiol group;
a structure (C) according to the above wherein the milk protein 1 is a milk protein comprising a free thiol group, the milk protein 2 is a milk protein comprising a free thiol group, the milk protein 3 is a milk protein not comprising a free thiol group, and the milk protein 4 is a milk protein not comprising a free thiol group;
a structure (C) according to the above wherein the milk protein 1 is a milk protein comprising a free thiol group, the milk protein 2 is a milk protein not comprising a free thiol group, the milk protein 3 is a milk protein comprising a free thiol group, and the milk protein 4 is a milk protein not comprising a free thiol group;
a structure (C) according to the above wherein the milk protein 1 is a milk protein comprising a free thiol group, the milk protein 2 is a milk protein not comprising a free thiol group, the milk protein 3 is a milk protein not comprising a free thiol group, and the milk protein 4 is a milk protein comprising a free thiol group; or
a structure (C) according to the above wherein the milk protein 1 does not comprises a free thiol group, the milk protein 2 is a milk protein comprising a free thiol group, the milk protein 3 is a milk protein comprising a free thiol group, and the milk protein 4 is a milk protein not comprising a free thiol group.

Non-limiting examples of such recombinant proteins include:
a structure (A) according to the above having the form: (β-lactoglobulin)-(linker peptide 1) -(β-lactoglobulin);
a structure (A) according to the above having the form: (bovine serum albumin)-(linker peptide 1)-(bovine serum albumin);
a structure (A) according to the above having the form: (lactoperoxidase)-(linker peptide 1)-(lactoperoxidase);
a structure (A) according to the above having the form: (β-lactoglobulin)-(linker peptide 1)-(bovine serum albumin);
a structure (A) according to the above having the form: (β-lactoglobulin)-(linker peptide 1)-(lactoperoxidase);
a structure (A) according to the above having the form: (bovine serum albumin)-(linker peptide 1)-(lactoperoxidase);
a structure (B) according to the above having the form: (β-lactoglobulin)-(linker peptide 1) -(β-lactoglobulin)-(linker peptide 2)-(α-lactalbumin);
a structure (B) according to the above having the form: (bovine serum albumin)-(linker peptide 1)-(bovine serum albumin)-(linker peptide 2)-(α-lactalbumin);
a structure (B) according to the above having the form: (lactoperoxidase)-(linker peptide 1)-(lactoperoxidase)-(linker peptide 2)-(α-lactalbumin);
a structure (B) according to the above having the form: (β-lactoglobulin)-(linker peptide 1)-(bovine serum albumin)-(linker peptide 2)-(α-lactalbumin);
a structure (B) according to the above having the form: (β-lactoglobulin)-(linker peptide 1)-(lactoperoxidase)-(linker peptide 2)-(α-lactalbumin);
a structure (B) according to the above having the form: (bovine serum albumin)-(linker peptide 1)-(lactoperoxidase)-(linker peptide 2)-(α-lactalbumin);
a structure (B) according to the above having the form: (β-lactoglobulin)-(linker peptide 1) -(α-lactalbumin)-(linker peptide 2)-(β-lactoglobulin);
a structure (B) according to the above having the form: (bovine serum albumin)-(linker peptide 1)-(α-lactalbumin)-(linker peptide 2)-(bovine serum albumin);
a structure (B) according to the above having the form: (lactoperoxidase)-(linker peptide 1) -(α-lactalbumin)-(linker peptide 2)-(lactoperoxidase);
a structure (B) according to the above having the form: (β-lactoglobulin)-(linker peptide 1) -(α-lactalbumin)-(linker peptide 2)-(bovine serum albumin);
a structure (B) according to the above having the form: (β-lactoglobulin)-(linker peptide 1) -(α-lactalbumin)-(linker peptide 2)-(lactoperoxidase);
a structure (B) according to the above having the form: (bovine serum albumin)-(linker peptide 1)-(α-lactalbumin)-(linker peptide 2)-(lactoperoxidase);
a structure (C) according to the above having the form: (β-lactoglobulin)-(linker peptide 1) -(β-lactoglobulin)-(linker peptide 2)-(α-lactalbumin)-(linker peptide 3)-(α-lactalbumin);
a structure (C) according to the above having the form: (bovine serum albumin)-(linker peptide 1)-(bovine serum albumin)-(linker peptide 2)-(α-lactalbumin)-(linker peptide 3) -(α-lactalbumin);
a structure (C) according to the above having the form: (lactoperoxidase)-(linker peptide 1)-(lactoperoxidase)-(linker peptide 2)-(α-lactalbumin)-(linker peptide 3)-(α-lactalbumin);
a structure (C) according to the above having the form: (β-lactoglobulin)-(linker peptide 1)-(bovine serum albumin)-(linker peptide 2)-(α-lactalbumin)-(linker peptide 3) -(α-lactalbumin);
a structure (C) according to the above having the form: (β-lactoglobulin)-(linker peptide 1)-(lactoperoxidase)-(linker peptide 2)-(α-lactalbumin)-(linker peptide 3)-(α-lactalbumin);
a structure (C) according to the above having the form: (bovine serum albumin)-(linker peptide 1)-(β-lactoglobulin)-(linker peptide 2)-(α-lactalbumin)-(linker peptide 3) -(α-lactalbumin);
a structure (C) according to the above having the form: (bovine serum albumin)-(linker peptide 1)-(lactoperoxidase)-(linker peptide 2)-(α-lactalbumin)-(linker peptide 3) -(α-lactalbumin);
a structure (C) according to the above having the form: (lactoperoxidase)-(linker peptide 1) -(β-lactoglobulin)-(linker peptide 2)-(α-lactalbumin)-(linker peptide 3)-(α-lactalbumin);
a structure (C) according to the above having the form: (lactoperoxidase)-(linker peptide 1)-(bovine serum albumin)-(linker peptide 2)-(α-lactalbumin)-(linker peptide 3) -(α-lactalbumin);
a structure (C) according to the above having the form: (β-lactoglobulin)-(linker peptide 1) -(α-lactalbumin)-(linker peptide 2)-(β-lactoglobulin)-(linker peptide 3)-(α-lactalbumin);
a structure (C) according to the above having the form: (bovine serum albumin)-(linker peptide 1)-(α-lactalbumin)-(linker peptide 2)-(bovine serum albumin)-(linker peptide 3) -(α-lactalbumin);
a structure (C) according to the above having the form: (lactoperoxidase)-(linker peptide 1) -(α-lactalbumin)-(linker peptide 2)-(lactoperoxidase)-(linker peptide 3)-(α-lactalbumin);
a structure (C) according to the above having the form: (β-lactoglobulin)-(linker peptide 1) -(α-lactalbumin)-(linker peptide 2)-(bovine serum albumin)-(linker peptide 3) -(α-lactalbumin);
a structure (C) according to the above having the form: (β-lactoglobulin)-(linker peptide 1) -(α-lactalbumin)-(linker peptide 2)-(lactoperoxidase)-(linker peptide 3)-(α-lactalbumin);
a structure (C) according to the above having the form: (bovine serum albumin)-(linker peptide 1)-(α-lactalbumin)-(β-lactoglobulin)-(bovine serum albumin)-(linker peptide 3) -(α-lactalbumin);
a structure (C) according to the above having the form: (bovine serum albumin)-(linker peptide 1)-(α-lactalbumin)-(linker peptide 2)-(lactoperoxidase)-(linker peptide 3) -(α-lactalbumin);
a structure (C) according to the above having the form: (lactoperoxidase)-(linker peptide 1) -(α-lactalbumin)-(linker peptide 2)-(β-lactoglobulin)-(linker peptide 3)-(α-lactalbumin);
a structure (C) according to the above having the form: (lactoperoxidase)-(linker peptide 1) -(α-lactalbumin)-(linker peptide 2)-(bovine serum albumin)-(linker peptide 3) -(α-lactalbumin);
a structure (C) according to the above having the form: (β-lactoglobulin)-(linker peptide 1) -(α-lactalbumin)-(linker peptide 2)-(α-lactalbumin)-(linker peptide 3)-(β-lactoglobulin);
a structure (C) according to the above having the form: (bovine serum albumin)-(linker peptide 1)-(α-lactalbumin)-(linker peptide 2)-(α-lactalbumin)-(linker peptide 3)-(bovine serum albumin);
a structure (C) according to the above having the form: (lactoperoxidase)-(linker peptide 1) -(α-lactalbumin)-(linker peptide 2)-(α-lactalbumin)-(linker peptide 3)-(lactoperoxidase);
a structure (C) according to the above having the form: (β-lactoglobulin)-(linker peptide 1) -(α-lactalbumin)-(linker peptide 2)-(α-lactalbumin)-(linker peptide 3)-(bovine serum albumin);
a structure (C) according to the above having the form: (β-lactoglobulin)-(linker peptide 1) -(α-lactalbumin)-(linker peptide 2)-(α-lactalbumin)-(linker peptide 3)-(lactoperoxidase);
a structure (C) according to the above having the form: (bovine serum albumin)-(linker peptide 1)-(α-lactalbumin)-(linker peptide 2)-(α-lactalbumin)-(linker peptide 3)-(lactoperoxidase);
a structure (C) according to the above having the form: (α-lactalbumin)-(linker peptide 1) -(β-lactoglobulin)-(linker peptide 2)-(β-lactoglobulin)-(linker peptide 3) -(α-lactalbumin);
a structure (C) according to the above having the form: (α-lactalbumin)-(linker peptide 1)-(bovine serum albumin)-(linker peptide 2)-(bovine serum albumin)-(linker peptide 3) -(α-lactalbumin);
a structure (C) according to the above having the form: (α-lactalbumin)-(linker peptide 1)-(lactoperoxidase)-(linker peptide 2)-(lactoperoxidase)-(linker peptide 3)-(α-lactalbumin);
a structure (C) according to the above having the form: (α-lactalbumin)-(linker peptide 1) -(β-lactoglobulin)-(linker peptide 2)-(bovine serum albumin)-(linker peptide 3) -(α-lactalbumin);
a structure (C) according to the above having the form: (α-lactalbumin)-(linker peptide 1) -(β-lactoglobulin)-(linker peptide 2)-(lactoperoxidase)-(linker peptide 3) -(α-lactalbumin); and
a structure (C) according to the above having the form: (α-lactalbumin)-(linker peptide 1)-(bovine serum albumin)-(linker peptide 2)-(lactoperoxidase)-(linker peptide 3) -(α-lactalbumin).

In a recombinant protein having a structure (B) or (C) according to the above, just three of the milk protein 1, the milk protein 2, the milk protein 3, and the milk protein 4 may be a milk protein comprising a free thiol group (i.e., the recombinant protein comprises trivalent free thiol groups).

Accordingly, the recombinant protein may have:
a structure (B) according to the above wherein the milk protein 1 is a milk protein comprising a free thiol group, the milk protein 2 is a milk protein comprising a free thiol group, and the milk protein 3 is a milk protein comprising a free thiol group;
a structure (C) according to the above wherein the milk protein 1 is a milk protein comprising a free thiol group, the milk protein 2 is a milk protein comprising a free thiol group, the milk protein 3 is a milk protein comprising a free thiol group, and the milk protein 4 is a milk protein not comprising a free thiol group; or
a structure (C) according to the above wherein the milk protein 1 is a milk protein comprising a free thiol group, the milk protein 2 is a milk protein comprising a free thiol group, the milk protein 3 is a milk protein not comprising a free thiol group, and the milk protein 4 is a milk protein comprising a free thiol group.

Non-limiting examples of such recombinant proteins include:
a structure (B) according to the above having the form: (β-lactoglobulin)-(linker peptide 1)-(β-lactoglobulin)-(linker peptide 2)-(β-lactoglobulin);
a structure (B) according to the above having the form: (bovine serum albumin)-(linker peptide 1)-(bovine serum albumin)-(linker peptide 2)-(bovine serum albumin);
a structure (B) according to the above having the form: (lactoperoxidase)-(linker peptide 1)-(lactoperoxidase)-(linker peptide 2)-(lactoperoxidase);
a structure (B) according to the above having the form: (β-lactoglobulin)-(linker peptide 1)-(β-lactoglobulin)-(linker peptide 2)-(bovine serum albumin);
a structure (B) according to the above having the form: (β-lactoglobulin)-(linker peptide 1)-(β-lactoglobulin)-(linker peptide 2)-(lactoperoxidase);
a structure (B) according to the above having the form: (bovine serum albumin)-(linker peptide 1)-(bovine serum albumin)-(linker peptide 2)-(β-lactoglobulin);
a structure (B) according to the above having the form: (bovine serum albumin)-(linker peptide 1)-(bovine serum albumin)-(linker peptide 2)-(lactoperoxidase);
a structure (B) according to the above having the form: (lactoperoxidase)-(linker peptide 1)-(lactoperoxidase)-(linker peptide 2)-(β-lactoglobulin);
a structure (B) according to the above having the form: (lactoperoxidase)-(linker peptide 1)-(lactoperoxidase)-(linker peptide 2)-(bovine serum albumin);
a structure (B) according to the above having the form: (β-lactoglobulin)-(linker peptide 1)-(bovine serum albumin)-(linker peptide 2)-(β-lactoglobulin);
a structure (B) according to the above having the form: (β-lactoglobulin)-(linker peptide 1)-(lactoperoxidase)-(linker peptide 2)-(β-lactoglobulin);
a structure (B) according to the above having the form: (bovine serum albumin)-(linker peptide 1)-(β-lactoglobulin)-(linker peptide 2)-(bovine serum albumin);
a structure (B) according to the above having the form: (bovine serum albumin)-(linker peptide 1)-(lactoperoxidase)-(linker peptide 2)-(bovine serum albumin);
a structure (B) according to the above having the form: (lactoperoxidase)-(linker peptide 1)-(β-lactoglobulin)-(linker peptide 2)-(lactoperoxidase);
a structure (B) according to the above having the form: (lactoperoxidase)-(linker peptide 1)-(bovine serum albumin)-(linker peptide 2)-(lactoperoxidase);
a structure (B) according to the above having the form: (β-lactoglobulin)-(linker peptide 1)-(bovine serum albumin)-(linker peptide 2)-(lactoperoxidase);
a structure (B) according to the above having the form: (bovine serum albumin)-(linker peptide 1)-(lactoperoxidase)-(linker peptide 2)-(β-lactoglobulin);
a structure (B) according to the above having the form: (lactoperoxidase)-(linker peptide 1)-(β-lactoglobulin)-(linker peptide 2)-(bovine serum albumin);
a structure (C) according to the above having the form: (β-lactoglobulin)-(linker peptide 1)-(β-lactoglobulin)-(linker peptide 2)-(β-lactoglobulin)-(linker peptide 3) -(α-lactalbumin);
a structure (C) according to the above having the form: (β-lactoglobulin)-(linker peptide 1)-(β-lactoglobulin)-(linker peptide 2)-(bovine serum albumin)-(linker peptide 3) -(α-lactalbumin);
a structure (C) according to the above having the form: (β-lactoglobulin)-(linker peptide 1)-(bovine serum albumin)-(linker peptide 2)-(lactoperoxidase)-(linker peptide 3) -(α-lactalbumin);
a structure (C) according to the above having the form: (lactoperoxidase)-(linker peptide 1)-(β-lactoglobulin)-(linker peptide 2)-(β-lactoglobulin)-(linker peptide 3) -(α-lactalbumin);
a structure (C) according to the above having the form: (lactoperoxidase)-(linker peptide 1)-(β-lactoglobulin)-(linker peptide 2)-(bovine serum albumin)-(linker peptide 3) -(α-lactalbumin);
a structure (C) according to the above having the form: (lactoperoxidase)-(linker peptide 1)-(β-lactoglobulin)-(linker peptide 2)-(lactoperoxidase)-(linker peptide 3) -(α-lactalbumin);
a structure (C) according to the above having the form: (lactoperoxidase)-(linker peptide 1)-(lactoperoxidase)-(linker peptide 2)-(lactoperoxidase)-(linker peptide 3) -(α-lactalbumin);
a structure (C) according to the above having the form: (β-lactoglobulin)-(linker peptide 1)-(β-lactoglobulin)-(linker peptide 2)-(α-lactalbumin)-(linker peptide 3)-(lactoperoxidase);
a structure (C) according to the above having the form: (β-lactoglobulin)-(linker peptide 1)-(lactoperoxidase)-(linker peptide 2)-(α-lactalbumin)-(linker peptide 3) -(β-lactoglobulin);
a structure (C) according to the above having the form: (β-lactoglobulin)-(linker peptide 1)-(lactoperoxidase)-(linker peptide 2)-(α-lactalbumin)-(linker peptide 3)-(bovine serum albumin);
a structure (C) according to the above having the form: (β-lactoglobulin)-(linker peptide 1)-(lactoperoxidase)-(linker peptide 2)-(α-lactalbumin)-(linker peptide 3)-(lactoperoxidase);
a structure (C) according to the above having the form: (bovine serum albumin)-(linker peptide 1)-(β-lactoglobulin)-(linker peptide 2)-(α-lactalbumin)-(linker peptide 3)-(lactoperoxidase);
a structure (C) according to the above having the form: (bovine serum albumin)-(linker peptide 1)-(lactoperoxidase)-(linker peptide 2)-(α-lactalbumin)-(linker peptide 3) -(β-lactoglobulin);
a structure (C) according to the above having the form: (lactoperoxidase)-(linker peptide 1)-(bovine serum albumin)-(linker peptide 2)-(α-lactalbumin)-(linker peptide 3)-(lactoperoxidase); and
a structure (C) according to the above having the form: (lactoperoxidase)-(linker peptide 1)-(lactoperoxidase)-(linker peptide 2)-(α-lactalbumin)-(linker peptide 3)-(β-lactoglobulin).

In a recombinant protein having a structure (C) according to the above, all (i.e., four) of the milk protein 1, the milk protein 2, the milk protein 3, and the milk protein 4 may be a milk protein comprising a free thiol group (i.e., the recombinant protein comprises tetravalent free thiol groups).

Accordingly, the recombinant protein may have a structure (C) according to the above wherein the milk protein 1 is a milk protein comprising a free thiol group, the milk protein 2 is a milk protein comprising a free thiol group, the milk protein 3 is a milk protein comprising a free thiol group, and the milk protein 4 is a milk protein comprising a free thiol group.

Non-limiting examples of such recombinant proteins include:
a structure (C) according to the above having the form: (β-lactoglobulin)-(linker peptide 1)-(β-lactoglobulin)-(linker peptide 2)-(β-lactoglobulin)-(linker peptide 3) -(β-lactoglobulin);
a structure (C) according to the above having the form: (bovine serum albumin)-(linker peptide 1)-(bovine serum albumin)-(linker peptide 2)-(bovine serum albumin)-(linker peptide 3)-(bovine serum albumin);
a structure (C) according to the above having the form: (lactoperoxidase)-(linker peptide 1)-(lactoperoxidase)-(linker peptide 2)-(lactoperoxidase)-(linker peptide 3)-(lactoperoxidase);
a structure (C) according to the above having the form: (β-lactoglobulin)-(linker peptide 1)-(β-lactoglobulin)-(linker peptide 2)-(lactoperoxidase)-(linker peptide 3)-(lactoperoxidase);
a structure (C) according to the above having the form: (β-lactoglobulin)-(linker peptide 1)-(lactoperoxidase)-(linker peptide 2)-(lactoperoxidase)-(linker peptide 3)-(lactoperoxidase); and
a structure (C) according to the above having the form: (β-lactoglobulin)-(linker peptide 1)-(β-lactoglobulin)-(linker peptide 2)-(β-lactoglobulin)-(linker peptide 3)-(bovine serum albumin).

In a recombinant protein having a structure (A), (B), or (C) according to the above, the milk protein 1, the milk protein 2, the milk protein 3, and/or the milk protein 4 may have a native PTM (e.g., a mammalian PTM), a non-native PTM (e.g., a non-mammalian PTM), or a mixture of at least one native PTM and at least one non-native PTM. The term “non-native PTM” as used herein refers to a difference in one or more location(s) of one or more PTMs (e.g., glycosylation, phosphorylation) in a protein, and/or a difference in the type of one or more PTMs at one or more location(s) in a protein compared to the protein as it exists in nature (e.g., in a mammal-derived milk; i.e., the protein having “native PTMs”).

### Linker Peptides

In the recombinant protein according to any of the above, the linker peptide 1, the linker peptide 2, and/or the linker peptide 3 may have a length of between 0 and about 80 amino acids (e.g., between 0 and 80, 70, 60, 50, 40, 30, 20, 10, or 5 amino acids; between 5 and 80, 70, 60, 50, 40, 30, 20, or 10 amino acids; between 10 and 80, 70, 60, 50, 40, 30, or 20 amino acids; between 20 and 80, 70, 60, 50, 40, or 30 amino acids; between 30 and 80, 70, 60, 50, or 40 amino acids; between 40 and 80, 70, 60, or 50 amino acids; between 50 and 80, 70, or 60 amino acids; between 60 and 80, or 70 amino acids; or between 70 and 80 amino acids).

In the recombinant protein according to any of the above, the linker peptide 1, the linker peptide 2, and/or the linker peptide 3 may comprise one or more free thiol groups. The one or more linker peptides may include one or more cysteine residues. Alternatively, the linker peptide 1, the linker peptide 2, and/or the linker peptide 3 may be devoid of any cysteine residues, or may be devoid of any free thiol groups.

In the recombinant protein according to any of the above, the linker peptide 1, the linker peptide 2, and/or the linker peptide 3 may comprise a protease recognition or cleavage sequence. Non-limiting examples of suitable protease recognition or cleavage sequences include recognition or cleavage sequences for any of the proteases disclosed herein. Alternatively, the linker peptide 1, the linker peptide 2, and/or the linker peptide 3 may comprise one or more amino acids that, when glycosylated, provide protection against proteolytic degradation (e.g., serine residues, threonine residues).

In the recombinant protein according to any of the above, the linker peptide 1, the linker peptide 2, and/or the linker peptide 3 may stabilize an emulsion.

In the recombinant protein according to any of the above, the linker peptide 1, the linker peptide 2, and/or the linker peptide 3 may comprise one or more amino acids (such as, for example, glycerin residues) that promote structural flexibility, or may comprise one or more amino acids that promote structural rigidity.

In the recombinant protein according to any of the above, the linker peptide 1, the linker peptide 2, and/or the linker peptide 3 may be a synthetic linker (i.e., a linker that is not found in nature). Synthetic linkers are known in the art (see, for example, Reddy Chichili et al. 2013 Protein Sci 22:153-67, Zou et al. 2012 Microb Cell Fact 11:21, Gustavsson et al. 2001 Protein engineering 14(9):711-715, Adlakha et al. 2011 Appl Environ Microbiol 77(14):4859-4866, and Adlakha et al. 2011 Appl Environ Microbiol 77(14):4859-4866).

In the recombinant protein according to any of the above, the linker peptide 1, the linker peptide 2, and/or the linker peptide 3 may be a native linker (i.e., a linker that is found in nature). Native linkers are known in the art (see, for example, Beckham et al. 2010 Biophysical Journal 99(11):3773-3781, Levasseur et al. 2005 Applied & Environmental Microbiology 71(12):8132-8140, Tan et al. 2015 Nat Commun 6: 7542, Brunecky et al. 2013 Science 342(6165):1513-1516, Kahn et al. 2019 Biotechnol Biofuels 12:44, Conesa et al. 2001 FEBS Lett. 503(2-3):117-120).

### Property of Recombinant Protein

A recombinant protein according to any of the above may have various properties. Non-limiting examples of properties include solubility (i.e., amount of protein that is dispersed and colloidally stable under given conditions and in various solvents; e.g., a solubility that is similar to or greater than that of a whey protein or of a potato protein), foaming/leavening capacity (i.e., capacity to form a foam, wherein the term “foam” as used herein refers to air bubbles dispersed in a solid or aqueous continuous phase), foam stability (i.e., half-life of foam in response to a physical and/or chemical condition), emulsifying capacity (i.e., capacity to stabilize an emulsion or the amount of oil that a given mass of sample can emulsify without destabilization), gelling/thickening/coagulating capacity (i.e., capacity to form a gel, wherein the term “gel” as used herein refers to a protein network with spaces filled with solvent linked by hydrogen bonds to the protein molecules and having defined viscoelastic properties), gelling profile (e.g., curve of gelling/thickening/coagulating capacity over time, viscoelastic parameters as a function of temperature), gel strength (i.e., mechanical force required to break a gel surface of a defined area)) (e.g., a gelling/thickening/coagulating behavior that is similar to that of gelatin), water binding capacity (i.e., amount of water bound by a defined mass of protein), heat stability (i.e., structural stability at elevated temperature at various pHs and ionic strengths; e.g. a heat stability that is similar to that of β-lactoglobulin, a heat stability that is at least 80° C.), shelf stability, freeze/thaw stability, health profile (e.g., is essentially free of an allergenic epitope [i.e., an amino acid sequence that elicits an allergenic response in a human or other animal], is essentially free of an inflammatory T-cell epitope), gastro-intestinal digestibility (i.e., rate at which an agent is degraded in a human or other animal gastrointestinal tract (i.e., mouth, esophagus, stomach, small intestine, large intestine, and anus), heat stability in fermentation broth (i.e., percentage of full-length protein products in the fermentation broths), and taste (e.g., a bland taste, lack of flavor). For example, a recombinant protein having a structure (A), (B), or (C) according to the above, wherein at least one of the milk protein 1, the milk protein 2, the milk protein 3, and the milk protein 4 is a β-lactoglobulin comprising a free thiol group, and at least one of the milk protein 1, the milk protein 2, the milk protein 3, and the milk protein 4 is a casein (e.g., a κ-casein, a 3-casein, an α-S1-casein, an α-S2-casein), can be produced and/or secreted at a higher titer by a recombinant host cell, or have a greater stability (e.g., protein stability in a fermentation of the recombinant host cell), compared to the casein without covalent linkage to the β-lactoglobulin(s).

### Recombinant Expression Construct

In various aspects, provided herein is a recombinant expression construct consisting of a polynucleotide comprising:
a promoter sequence (e.g., a polynucleotide sequence for any of the promoters disclosed herein),
an optional secretion signal sequence (i.e., a sequence that encodes a peptide that mediates the delivery of a nascent protein attached to the peptide to the exterior of the cell in which the nascent protein is synthesized; e.g., a polynucleotide sequence encoding any of the secretion signals disclosed herein),
a recombinant protein coding sequence (i.e., a polynucleotide sequence encoding the recombinant protein according to any of the above, optionally comprising a tag polypeptide (e.g., any of the tag polypeptides disclosed herein)), and
a termination sequence (e.g., a polynucleotide sequence for any of the terminators disclosed herein);
   wherein:
the promoter sequence is operably linked in sense orientation to the optional secretion signal sequence and the recombinant protein coding sequence (i.e., the promoter sequence and the optional secretion signal sequence and the recombinant protein coding sequence are positioned such that the promoter sequence is effective in mediating or regulating transcription of the optional secretion signal sequence and the recombinant protein coding sequence),
the optional secretion signal sequence is operably linked in sense orientation to the recombinant protein coding sequence (i.e., the optional secretion signal sequence and the recombinant protein coding sequence are positioned such that transcription and translation produces a recombinant protein comprising the optional secretion signal), and
the one or more terminator sequences are operably linked to the recombinant protein coding sequence (i.e., the recombinant protein coding sequence and the one or more terminator sequences are positioned such that the one or more terminator sequences are effective in terminating transcription of the recombinant protein coding sequence).

The recombinant expression construct may further comprise an operably linked sequence encoding for an affinity purification tag, such that the expressed recombinant protein includes a peptide sequence for affinity purification. Such affinity purification tag may be operably linked such that when expressed the affinity purification tag is present either at or toward the amino terminus, the carboxy terminus, or both. Such affinity purification tag may comprise a maltose binding protein (MBP) tag, a glutathione-S-transferase (GST) tag, a poly(His) tag, a hexa(His) tag, a FLAG-tag, a V5-tag, a VSV-tag, an E-tag, an NE-tag, a hemagglutinin (Ha)-tag, and a Myc-tag.

The recombinant expression construct may further comprise a sequence for integration by homologous (i.e., targeted integration) or nonhomologous recombination into the genome of a host cell. The recombinant expression construct may comprise at least 10, at least 25, at least 50, at least 100, at least 250, at least 500, at least 750, at least 1,000, or at least 10,000 base pairs that have sufficient identity with a target sequence in the genome of the host cell to enhance the probability of homologous recombination of the recombinant expression construct. Such homologous sequence may be non-coding or coding.

The optional secretion signal sequence and/or recombinant protein coding sequence comprised in the recombinant expression construct according to any of the above may be codon-optimized for expression in the recombinant host cell according to any of the above.

The recombinant expression construct according to any of the above may be isolated.

The recombinant expression construct according to any of the above may be generated upon integration of a fragment of the recombinant expression construct into the genome of a host cell (e.g., the genome of the recombinant host cell according to any of the above). For example, a polynucleotide comprising a recombinant protein coding sequence (optionally operably linked to a secretion signal sequence) may be stably integrated within the genome of a host cell such that one or more regulatory elements of an endogenous gene locus become operably linked to the recombinant protein coding sequence, thereby generating the recombinant expression construct according to any of the above.

### Promoter Sequence

The recombinant expression construct according to any of the above may comprise any promoter sequence that is active in a recombinant host cell according to any of the below.

The promoter sequence may be a constitutive promoter sequence (i.e., a promoter sequence that is active under most environmental and developmental conditions), or an inducible or repressible promoter sequence (i.e., a promoter sequence that is active only under certain environmental or developmental conditions [e.g., in presence or absence of certain factors, such as, but not limited to, carbon (e.g., glucose, galactose, lactose, sucrose, cellulose, sophorose, gentiobiose, sorbose, disaccharides that induce the cellulase promoters, starch, tryptophan, thiamine, methanol), phosphate, nitrogen, or other nutrient; temperature; pH; osmolarity; heavy metals or heavy metal ions; inhibitors; stress; catabolites; and combinations thereof]).

The promoter sequence may consist of a single promoter sequence, or of two or more promoter sequences (e.g., combination of two or more promoters or functional parts thereof arranged in sequence, combination of an inducible and a constitutive promoter). The two or more promoter sequences may be identical, or at least two of the two or more promoter sequences cannot be identical.

The promoter sequence may comprise or consist of a bidirectional promoter sequence (i.e., a polynucleotide that initiates transcription in both orientations by recruiting transcription factors, for example generated by fusing two identical or different promoters in opposite directions).

Non-limiting examples of suitable promoter sequences include promoter sequences that are functional in a bacterial host cell, including T7 promoter, T5 promoter, Tac promoter, pL/pR promoter, phoA promoter, lacUV5 promoter, trc promoter, trp promoter, cstA promoter, xylA promoter, manP promoter, malA promoter, lacA promoter, aprE promoter, AaprE promoter, srfA promoter, p43 promoter, ylbA promoter, 6B promoter, veg promoter, PG1 promoter, PG6 promoter, kPL promoter, kPR promoter, and spa promoter, and functional parts and combinations thereof.

Non-limiting examples of suitable promoter sequences include promoter sequences that are functional in a fungal host cell, including xlnA promoter, xyn1 promoter, xyn2 promoter, xyn3 promoter, xyn4 promoter, bxl1 promoter, cbh1 promoter, cbh2 promoter, egl1 promoter, egl2 promoter, egl3 promoter, egl4 promoter, egl5 promoter, glaA promoter, agdA promoter, gpdA promoter, gpd1 promoter, AOX1 promoter, GAP1 promoter, MET3 promoter, ENO1 promoter, GPD1 promoter, PDC1 promoter, TEF1 promoter, AXE1 promoter, CIP1 promoter, GH61 promoter, PKI1 promoter, RP2 promoter, ADH1 promoter, CUP1 promoter, GAL1 promoter, PGK1 promoter, YPT1 promoter, LAC4 promoter, LAC4-PB1 promoter, FLD1 promoter, MOX promoter, DAS1 promoter, DAS2 promoter, GAP1 promoter, STR3 promoter, ADH3 promoter, GUT2 promoter, CYC1 promoter, TDH3 promoter, PGL1 promoter, ADH2 promoter, HXT7 promoter, CLB1 promoter, and PHO5 promoter, and functional parts and combinations thereof.

### Secretion Signal Sequence

The recombinant expression construct according to any of the above may optionally comprise any secretion signal sequence that is active in a recombinant host cell according to any of the below.

The optional secretion signal sequence may encode a secretion signal that mediates translocation of the nascent recombinant protein into the ER post-translationally (i.e., protein synthesis precedes translocation such that the nascent recombinant protein is present in the cell cytosol prior to translocating into the ER) or co-translationally (i.e., protein synthesis and translocation into the ER occur simultaneously).

Non-limiting examples of suitable secretion signal sequences include secretion signal sequences that are functional in a bacterial host cell, including secretion signal sequences of genes encoding any of the following proteins: PelB, OmpA, Bla, PhoA, PhoS, MalE, LivK, LivJ, MglB, AraF, AmpC, RbsB, MerP, CpdB, Lpp, LamB, OmpC, PhoE, OmpF, TolC, BtuB, and LutA, and functional parts and combinations thereof.

Non-limiting examples of suitable secretion signal sequences include secretion signal sequences that are functional in a fungal host cell, including secretion signal sequences of genes encoding any of the following proteins: CBH1, CBH2, EGL1, EGL2, XYN1, XYN2, BXL1, HFB1, HFB2, GLAA, AMYA, AMYC, AAMA, alpha mating factor, SUC2, PHO5, INV, AMY, LIP, PIR, OST1, and β-glucosidase, and functional parts and combinations thereof.

### Termination Sequence

The recombinant expression construct according to any of the above may comprise any termination sequence that is active in a recombinant host cell according to any of the below.

Non-limiting examples of suitable termination sequences include termination sequences include termination sequences of the adh1, amaA, amdS, amyA, aox1, cbh1, cbh2, cycl, egl1, egl2, gal1, gap1, glaA, gpd1, gpdA, pdc1, pgk1 tef1, tps1, trpC, xyn1, xyn2, xyn3, and xyn4 genes, and functional parts and combinations thereof.

The termination sequence may consist of a single termination sequence, or of two or more termination sequences, wherein the two or more termination sequences may be identical, or at least two of the two or more termination sequences may be not identical. The termination sequence may consist of a bidirectional termination sequence.

### Additional Regulatory Elements

The recombinant expression construct according to any of the above may further comprise additional regulatory elements.

Non-limiting examples of regulatory elements include promoter sequences, termination sequences, transcriptional start sequences, translational start sequences, translation stop sequences, enhancer sequences, activator sequences, response elements, protein recognition sites, inducible elements, protein binding sequences, 5′ and 3′ untranslated regions (e.g., a 3′ untranslated region comprising a poly-adenylation signal), upstream activation sequences (UAS), introns, operators (i.e., sequences of nucleic acids adjacent to a promoter that comprise a protein-binding domain where a repressor protein can bind and reduce or eliminate activity of the promoter), efficient RNA processing signals (e.g., splicing signals, polyadenylation signals), sequences that stabilize cytoplasmic mRNA, sequences that enhance translation efficiency (e.g., ribosome binding sites [e.g., Shine-Dalgarno sequences]), sequences that enhance protein stability, sequences that enhance protein secretion, and combinations thereof.

### Recombinant Vector

In various aspects, provided herein is a recombinant vector that comprises the recombinant expression construct according to any of the above or a fragment thereof (e.g., a polynucleotide that comprises a recombinant protein coding sequence and optional secretion signal sequence, which upon integration into the genome of a host cell creates the recombinant expression construct according to any of the above).

The recombinant vector may comprise a single recombinant expression construct according to any of the above, or two or more recombinant expression constructs according to any of the above, which may be identical or at least two of which may be not identical (e.g., differ from each other in a promoter sequence, a secretion signal, a protein coding sequence, a termination sequence, and/or an additional regulatory element). In embodiments in which the recombinant vector comprises two or more recombinant expression constructs, the two or more recombinant expression constructs may encode the same recombinant protein. In some such embodiments, the two or more recombinant expression constructs encoding the same recombinant protein differ from each other in a promoter sequence, secretion signal sequence, termination sequence, and/or additional regulatory element.

The recombinant vector may further comprise one or more other elements suitable for propagation of the recombinant vector in a recombinant host cell. Non-limiting examples of such other elements include origins of replication and selection markers. Origins of replication and selection markers are known in the art, and include bacterial and fungal origins of replication (e.g., AMA1, ANSI). Selection markers may be resistance genes (i.e., polynucleotides that encode proteins that enable host cells to detoxify an exogenously added compound [e.g., an antibiotic compound]), auxotrophic markers (i.e., polynucleotides that encode proteins that permit a host cell to synthesize an essential component (usually an amino acid) while grown in media that lacks that essential component), or color markers (i.e., genes that encode proteins that can produce a color). Non-limiting examples of suitable selection markers include amdS (acetamidase), argB (ornithine carbamoyltransferase), bar (phosphinothricin acetyltransferase), hph (hygromycin phosphotransferase), niaD (nitrate reductase), pyrG (orotidine 5′-phosphate decarboxylase), sC (sulfate adenyltransferase), trpC (anthranilate synthase), and ble (bleomycin-type antibiotic resistance), and derivatives thereof. The selection marker may comprise an alteration that decreases production of the selective marker, thus increasing the number of copies needed to permit a recombinant host cell comprising the recombinant vector to survive under selection. Selection may also be accomplished by co-transformation, wherein the transformation is carried out with a mixture of two vectors and the selection is made for one vector only.

The recombinant vector may further comprise sequences for integration by homologous (i.e., targeted integration) or nonhomologous recombination into the genome of a host cell. The recombinant expression construct may comprise at least 10, at least 25, at least 50, at least 100, at least 250, at least 500, at least 750, at least 1,000, or at least 10,000 base pairs that have sufficient identity with a target sequence in the genome of the host cell to enhance the probability of homologous recombination of the recombinant expression construct. Such homologous sequence may be non-coding or coding.

The recombinant vector according to any of the above may be isolated.

### Recombinant Host Cell

In various aspects, provided herein is a recombinant host cell that is capable of producing the recombinant protein according to any of the above, wherein the recombinant host cell comprises a recombinant expression construct according to any of the above.

The recombinant host cell may comprise a single recombinant expression construct according to any of the above, or comprise two or more recombinant expression constructs according to any of the above. In embodiments in which the recombinant host cell comprises two or more recombinant expression constructs, the two or more recombinant expression constructs may be identical, or at least two of the two or more recombinant expression constructs may differ from each other (e.g., in a promoter sequence, a protein coding sequence, a secretion signal sequence, a termination sequence, and/or an additional regulatory element).

The recombinant host cell may comprise a recombinant expression construct that is stably integrated within the genome of the recombinant host cell (e.g., via targeted (e.g., via homologous recombination) or random (i.e., non-targeted) integration), and/or a recombinant expression construct that is not stably integrated but rather maintained extra-chromosomally (e.g., on an autonomously replicating recombinant vector provided herein).

The recombinant host cell according to any of the above may be derived from any organism, including any bacterium, fungus (e.g., yeast, filamentous fungus), archaea, protista, animal (including any unicellular animal), plant (including any unicellular plant), algae, protozoan, and chromista, or from a genetic variant (e.g., mutant) thereof, as well as from any generally recognized as safe (GRAS) industrial host cell.

Non-limiting examples of suitable plants include cycad, Ginkgo biloba, conifer, cypress, juniper, thuja, cedarwood, pine, angelica, caraway, coriander, cumin, fennel, parsley, dill, dandelion, helichrysum, marigold, mugwort, safflower, camomile, lettuce, wormwood, calendula, citronella, sage, thyme, chia seed, mustard, olive, coffee, capsicum, eggplant, paprika, cranberry, kiwi, vegetables (e.g., carrot, celery), tagete, tansy, tarragon, sunflower, wintergreen, basil, hyssop, lavender, lemon verbena, marjoram, melissa, patchouli, pennyroyal, peppermint, rosemary, sesame, spearmint, primrose, samara, pepper, pimento, potato, sweet potato, tomato, blueberry, nightshade, petunia, morning glory, lilac, jasmin, honeysuckle, snapdragon, psyllium, wormseed, buckwheat, amaranth, chard, quinoa, spinach, rhubarb, jojoba, cypselea, chlorella, marula, hazelnut, canola, kale, bok choy, rutabaga, frankincense, myrrh, elemi, hemp, pumpkin, squash, curcurbit, manioc, dalbergia, legume plants (e.g., alfalfa, lentil, bean, clover, pea, fava coceira, frijole bola roja, frijole negro, lespedeza, licorice, lupin, mesquite, carob, soybean, peanut, tamarind, wisteria, cassia, chickpea/garbanzo, fenugreek, green pea, yellow pea, snow pea, lima bean, fava bean), geranium, flax, pomegranate, cotton, okra, neem, fig, mulberry, clove, eucalyptus, tea tree, niaouli, fruiting plant (e.g., apple, apricot, peach, plum, pear, nectarine), strawberry, blackberry, raspberry, cherry, prune, rose, tangerine, citrus (e.g., grapefruit, lemon, lime, orange, bitter orange, mandarin, tangerine), mango, citrus bergamot, buchu, grape, broccoli, brussels sprout, camelina, cauliflower, rape, rapeseed (canola), turnip, cabbage, cucumber, watermelon, honeydew melon, zucchini, birch, walnut, cassava, baobab, allspice, almond, breadfruit, sandalwood, macadamia, taro, tuberose, aloe vera, garlic, onion, shallot, vanilla, yucca, vetiver, galangal, barley, corn, Curcuma aromatica, ginger, lemon grass, oat, palm, pineapple, rice, rye, sorghum, triticale, turmeric, yam, bamboo, barley, cajuput, canna, cardamom, maize, oat, wheat, cinnamon, sassafras, lindera benzoin, bay laurel, avocado, ylang-ylang, mace, nutmeg, moringa, horsetail, oregano, cilantro, chervil, chive, aggregate fruit, grain plant, herbal plant, leafy vegetable, non-grain legume plant, nut plant, succulent plant, land plant, water plant, delbergia, millet, drupe, schizocarp, flowering plant, non-flowering plant, cultured plant, wild plant, tree, shrub, flower, grass, herbaceous plant, brush, lianas, cacti, tropical plant, subtropical plant, temperate plant, and moss (e.g., Physcomitrella patens).

Non-limiting examples of suitable yeast include members of any of the following genera, and derivatives and crosses thereof: Candida (e.g., Candida albicans, Candida etchellsii, Candida guilliermondii, Candida humilis, Candida lipolytica, Candida orthopsilosis, Candida palmioleophila, Candida pseudotropicalis, Candida sp., Candida utilis, Candida versatilis), Cladosporium, Cryptococcus (e.g., Cryptococcus terricolus, Cryptococcus curvatus), Debaryomyces (e.g., Debaryomyces hansenii), Endomyces (e.g., Endomyces vernalis), Endomycopsis (e.g., Endomycopsis vernalis), Eremothecium (e.g., Eremothecium ashbyii), Hansenula (e.g., Hansenula sp., Hansenula polymorpha), Kluyveromyces (e.g., Kluyveromyces sp., Kluyveromyces lactis, Kluyveromyces marxianus var. lactis, Kluyveromyces marxianus, Kluyveromyces thermotolerans), Lipomyces (e.g., Lipomyces starkeyi, Lipomyecs lipofer), Ogataea (e.g., Ogataea minuta), Pichia (e.g., Pichia sp., Pichia pastoris (Komagataella phaffii), Pichia finlandica, Pichia trehalophila, Pichia koclamae, Pichia membranaefaciens, Pichia minuta, Pichia lindneri), Pichia opuntiae, Pichia thermotolerans, Pichia salictaria, Pichia guercuum, Pichia pijperi, Pichia stiptis, Pichia methanolica), Rhodosporidium (e.g., Rhodosporidium toruloides), Rhodotorula (e.g., Rhodotorula sp., Rhodotorula gracilis, Rhodotorula glutinis, Rhodotorula graminis), Saccharomyces (e.g., Saccharomyces sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum), Sporobolomyces (e.g., Sporobolomyces roseus), Sporidiobolus (e.g., Sporidiobolus johnsonii, Sporidiobolus salmonicolor), Trichosporon (e.g., Trichosporon cacaoliposimilis, Trichosporon oleaginosus sp. nov., Trichosporon cacaoliposimilis sp. nov., Trichosporon gracile, Trichosporon dulcitum, Trichosporon jirovecii, Trichosporon insectorum), Xanthophyllomyces (e.g., Xanthophyllomyces dendrorhous), Yarrowia (e.g., Yarrowia lipolytica), and Zygosaccharomyces (e.g., Zygosaccharomyces rouxii).

Non-limiting examples of suitable filamentous fungi include any holomorphic, teleomorphic, and anamorphic forms of fungi, including members of any of the following genera, and derivatives and crosses thereof: Acremonium (e.g., Acremonium alabamense), Aspergillus (e.g., Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus niger var. awamori, Aspergillus ochraceus, Aspergillus oryzae, Aspergillus sojae, Aspergillus terreus, as well as Emericella, Neosartorya, and Petromyces species), Aureobasidium, Canariomyces, Chaetomium, Chaetomidium, Corynascus, Chrysosporium (e.g., Chrysosporium botryoides, Chrysosporium carmichaeli, Chrysosporium crassitunicatum, Chrysosporium europae, Chrysosporium evolceannui, Chrysosporium farinicola, Chrysosporium fastidium, Chrysosporium filiforme, Chrysosporium georgiae, Chrysosporium globiferum, Chrysosporium globiferum var. articulatum, Chrysosporium globiferum var. niveum, Chrysosporium hirundo, Chrysosporium hispanicum, Chrysosporium holmii, Chrysosporium indicum, Chrysosporium iops, Chrysosporium keratinophilum, Chrysosporium kreiselii, Chrysosporium kuzurovianum, Chrysosporium lignorum, Chrysosporium obatum, Chrysosporium lucknowense, Chrysosporium lucknowense Garg 27K, Chrysosporium medium, Chrysosporium medium var. spissescens, Chrysosporium mephiticum, Chrysosporium merdarium, Chrysosporium merdarium var. roseum, Chrysosporium minor, Chrysosporium pannicola, Chrysosporium parvum, Chrysosporium parvum var. crescens, Chrysosporium pilosum, Chrysosporium pseudomerdarium, Chrysosporium pyriformis, Chrysosporium queenslandicum, Chrysosporium sigleri, Chrysosporium sulfureum, Chrysosporium synchronum, Chrysosporium tropicum, Chrysosporium undulatum, Chrysosporium vallenarense, Chrysosporium vespertilium, Chrysosporium zonatum), Coonemeria, Cunninghamella (e.g., Cunninghamella ehinulata), Dactylomyces, Emericella, Filibasidium, Fusarium (e.g., Fusarium moniliforme, Fusarium venenatum, Fusarium oxysporum, Fusarium graminearum, Fusarium proliferatum, Fusarium verticiollioides, Fusarium culmorum, Fusarium crookwellense, Fusarium poae, Fusarium sporotrichioides, Fusarium sambuccinum, Fusarium torulosum, as well as associated Gibberella teleomorphic forms thereof), Gibberella, Humicola, Hypocrea, Lentinula, Malbranchea (e.g., Malbranchea filamentosa), Magnaporthe, Malbranchium, Melanocarpus, Mortierella (e.g., Mortierella alpina 1S-4, Mortieralla isabelline, Mortierrla vinacea, Mortieralla vinaceae var. raffinoseutilizer), Mucor (e.g., Mucor miehei Cooney et Emerson (Rhizomucor miehei (Cooney & R. Emerson)) Schipper, Mucor pusillus Lindt, Mucor circinelloides Mucor mucedo), Myceliophthora (e.g., Myceliophthora thermophila), Myrothecium, Neocallimastix, Neurospora (e.g., Neurospora crassa), Paecilomyces, Penicillium (e.g., Penicillium chrysogenum, Pennicillium iilacinum, Penicillium roquefortii), Phenerochaete, Phlebia, Piromyces, Pythium, Rhizopus (e.g., Rhizopus niveus), Schizophyllum, Scytalidium, Sporotrichum (e.g., Sporotrichum cellulophilum), Stereum, Talaromyces, Thermoascus, Thermomyces, Thielavia (e.g., Thielavia terrestris), Tolypocladium, and Trichoderma (e.g., Trichoderma harzianum, Trichoderma koningii, Trichoderma longibrachiatum, Trichoderma reesei, Trichoderma atroviride, Trichoderma virens, Trichoderma citrinoviride, Trichoderma viride).

Non-limiting examples of suitable bacteria include firmicutes, cyanobacteria (blue-green algae), oscillatoriopheideae, bacillales, lactobacillales, oscillatoriales, bacillaceae, lactobacillaceae, and members of any of the following genera, and derivatives and crosses thereof: Acinetobacter, Acetobacter (e.g., Acetobacter suboxydans, Acetobacter xylinum), Actinoplane (e.g., Actinoplane missouriensis), Arthrospira (e.g., Arthrospira platensis, Arthrospira maxima), Bacillus (e.g., Bacillus cereus, Bacillus coagulans, Bacillus licheniformis, Bacillus stearothermophilus, Bacillus subtilis), Escherichia (e.g., Escherichia coli), Lactobacillus (e.g., Lactobacillus acidophilus, Lactobacillus bulgaricus), Lactococcus (e.g., Lactococcus lactis, Lactococcus lactis Lancefield Group N, Lactobacillus reuteri), Leuconostoc (e.g., Leuconostoc citrovorum, Leuconostoc dextranicum, Leuconostoc mesenteroides), Micrococcus (e.g., Micrococcus lysodeikticus), Rhodococcus (e.g., Rhodococcus opacus, Rhodococcus opacus strain PD630), Spirulina, Streptococcus (e.g., Streptococcus cremoris, Streptococcus lactis, Streptococcus lactis subspecies diacetylactis, Streptococcus thermophilus), Streptomyces (e.g., Streptomyces chattanoogensis, Streptomyces griseus, Streptomyces natalensis, Streptomyces olivaceus, Streptomyces olivochromogenes, Streptomyces rubiginosus), Tetrahymena (e.g., Tetrahymena thermophile, Tetrahymena hegewischi, Tetrahymena hyperangularis, Tetrahymena malaccensis, Tetrahymena pigmentosa, Tetrahymena pyriformis, Tetrahymena vorax), and Xanthomonas (e.g., Xanthomonas campestris).

Non-limiting examples of suitable algae include members of any of the following genera, and derivatives and crosses thereof: red algae, brown algae, green algae, microalgae, Achnanthes (e.g., Achnanthes orientalis), Agmenellum, Alaria (e.g., Alaria marginata), Amphiprora (e.g., Amphiprora hyaline), Amphora (e.g., Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp.), Anabaena, Analipus (e.g., Analipus japonicus), Ankistrodesmus (e.g., Ankistrodesmus falcatus), Ascophyllum (e.g., Ascophyllum nodosum), Boekelovia (e.g., Boekelovia hooglandii), Borodinella (e.g., Borodinella sp.), Botryococcus (e.g., Botryococcus braunii, Botryococcus sudeticus), Carteria, Chaetoceros (e.g., Chaetoceros gracilis, Chaetoceros muelleri, Chaetoceros muelleri subsalsum, Chaetoceros sp.), Chlorella (e.g., Chlorella anitrata, Chlorella Antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella protothecoides, Chlorella protothecoides var. acidicola, Chlorealla, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgarisf tertia, Chlorella vulgaris var. vulgaris f. viridis, Chlorella xanthella, Chlorella zofingiensis, Chlorella trebouxioides, Chlorella vulgaris), Chlorococcum (e.g., Chlorococcum infusionum, Chlorococcum sp.), Chlorogonium, Chondrus (e.g., Chondrus crispus, Chondrus ocellatus), Chroomonas (e.g., Chroomonas sp.), Chrysosphaera (e.g., Chrysosphaera sp.), Cricosphaera (e.g., Cricosphaera sp.), Cryptomonas (e.g., Cryptomonas sp.), Cyclotella (e.g., Cyclotella cryptica, Cyclotella meneghiniana, Cyclotella sp.), Dunaliella (e.g., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta), Ecklonia (e.g., Ecklonia sp), Eisenia (e.g., Eisenia bicyclis), Ellipsoidon (e.g., Ellipsoidon sp.), Eremosphaera (e.g., Eremosphaera viridis, Eremosphaera sp.), Eucheuma (e.g., Eucheuma cottonii, Eucheuma spinosum), Euglena, Fragilaria (e.g., Fragilaria crotonensis, Fragilaria sp.), Franceia (e.g., Franceia sp.), Furcellaria (e.g., Furcellaria fastigiate), Gigartina (e.g., Gigartina acicularis, Gigartina bursa-pastoris, Gigartina pistillata, Gigartina radula, Gigartina skottsbergii, Gigartina stellate), Gleocapsa (e.g., Gleocapsa sp.), Gloeothamnion (e.g., Gloeothamnion sp.), Gloiopeltis (e.g., Gloiopeltis furcate), Gracilaria (e.g., Gracilaria bursa-pastoris, Gracilaria lichenoides), Hizikia (e.g., Hizikia fusiforme), Hymenomonas (e.g., Hymenomonas sp.), Isochrysis (e.g., Isochrysis aff. galbana, Isochrysis galbana), Kjellmaniella (e.g., Kjellmaniella gyrate), Laminaria (e.g., Laminaria angustata, Laminaria longirruris, Laminaria Longissima, Laminaria ochotensis, Laminaria claustonia, Laminaria saccharina, Laminaria digitata, Laminaria japonica), Lepocinclis, Macrocystis (e.g., Macrocystis pyrifera), Micractinium, Monoraphidium (e.g., Monoraphidium minutum, Monoraphidium sp.), Nannochloris (e.g., Nannochloris sp.), Nannochloropsis (e.g., Nannochloropsis salina, Nannochloropsis sp.), Navicula (e.g., Navicula acceptata, Navicula biskanterae, Navicula pseudotenelloides, Navicula pelliculosa, Navicula saprophila, Navicula sp.), Nephrochloris (e.g., Nephrochloris sp.), Nephroselmis (e.g., Nephroselmis sp.), Nitzschia (e.g., Nitzschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp.), Ochromonas (e.g., Ochromonas sp.), Oocystis (e.g., Oocystis parva, Oocystis pusilla, Oocystis sp.), Oscillatoria (e.g., Oscillatoria limnetica, Oscillatoria sp., Oscillatoria subbrevis), Palmaria (e.g., Palmaria palmata), Pascheria (e.g., Pascheria acidophila), Pavlova (e.g., Pavlova sp.), Petalonia (e.g., Petalonia fascia), Phagus, Phormidium, Platymonas (e.g., Platymonas sp.), Pleurochrysis (e.g., Pleurochrysis carterae, Pleurochrysis dentate, Pleurochrysis sp.), Porphyra (e.g., Porphyra columbina, Porphyra crispata, Porhyra deutata, Porhyra perforata, Porhyra suborbiculata, Porphyra tenera), Porphyridium (e.g., Porphyridium cruentum, Porphyridium purpureum, Porphyridium aerugineum), Prototheca (e.g., Prototheca wickerhamii, Prototheca stagnora, Prototheca portoricensis, Prototheca moriformis, Prototheca zopfii), Pyramimonas (e.g., Pyramimonas sp.), Pyrobotrys, Rhodella (e.g., Rhodella maculate, Rhodella reticulata, Rhodella violacea), Rhodymenia (e.g., Rhodymenia palmata), Sarcinoid (e.g., Sarcinoid chrysophyte), Scenedesmus (e.g., Scenedesmus armatus), Scytosiphon (e.g., Scytosiphon lome), Spirogyra, Spirulina (e.g., Spirulina platensis), Stichococcus (e.g., Stichococcus sp.), Synechococcus (e.g., Synechococcus sp.), Tetraedron, Tetraselmis (e.g., Tetraselmis sp., Tetraselmis suecica), Thalassiosira (e.g., Thalassiosira weissflogii), and Viridiella (e.g., Viridiella fridericiana).

The recombinant host cell according to any of the above may comprise a genetic modification that improves production of the recombinant protein. Non-limiting examples of suitable genetic modifications include altered kinase activities, altered phosphatase activities, altered protease activities, altered gene expression induction pathways, altered production and/or activity of a protein involved in protein folding, and altered production and/or activity of a protein involved in protein secretion (e.g., vesicular transport).

### Method for Obtaining Recombinant Host Cell

In various aspects, provided herein is a method for obtaining the recombinant host cell according to any of the above, wherein the method comprises: obtaining a polynucleotide that encodes the recombinant protein (and optional secretion signal) according to any of the above, or the recombinant expression construct according to any of the above, or the recombinant vector according to any of the above; and introducing the polynucleotide, recombinant expression construct, or recombinant vector into a host cell (e.g., any of the host cells disclosed herein) to obtain the recombinant host cell according to any of the above.

The polynucleotide, recombinant expression construct, and/or recombinant vector may be obtained by any suitable method known in the art, including, without limitation, direct chemical synthesis and cloning.

Methods for introducing a polynucleotide, recombinant expression construct, or recombinant vector into a host cell are well-known in the art. Non-limiting examples of such methods include calcium phosphate transfection, dendrimer transfection, liposome transfection (e.g., cationic liposome transfection), cationic polymer transfection, DEAE-dextran transfection, cell squeezing, sonoporation, optical transfection, protoplast fusion, protoplast transformation, impalefection, hyrodynamic delivery, gene gun, magnetofection, viral transduction, electroporation, and chemical transformation (e.g., using PEG).

Methods for identifying a recombinant host cell are well-known in the art, and include screening for expression of a drug resistance or auxotrophic marker encoded by the polynucleotide, recombinant expression construct, or recombinant vector that permits selection for or against growth of cells, or by other means (e.g., detection of light emitting peptide comprised in the polynucleotide, recombinant expression construct, or recombinant vector, molecular analysis of individual recombinant host cell colonies [e.g., by restriction enzyme mapping, PCR amplification, Southern analysis, or sequence analysis of isolated extrachromosomal vectors or chromosomal integration sites]).

Production of the recombinant protein by the recombinant host cell according to any of the above may be evaluated using any suitable method known in the art, such as assays that are carried out at the RNA level and, most suitable, at the protein level, or by use of functional bioassays that measure the production or activity of the recombinant protein. Non-limiting examples of such assays include Northern blotting, dot blotting (DNA or RNA), RT-PCR (reverse transcriptase polymerase chain reaction), RNA-Seq, in situ hybridization, Southern blotting, enzyme activity assays, immunological assays (e.g., immunohistochemical staining, immunoassays, Western blotting, ELISA), and free thiol assays (e.g., for measuring production of protein comprising free cysteine residues).

### Method for Producing Recombinant Protein

In various aspects, provided herein is a method for producing the recombinant protein according to any of the above, wherein the method comprises: fermenting the recombinant host cell according to any of the above in a culture medium under conditions suitable for production of the recombinant protein.

The method may further comprise: purifying the recombinant protein from the fermentation broth to obtain a preparation comprising the recombinant protein; and/or post-processing the recombinant protein.

Alternatively, the recombinant protein may be obtained using in vitro methods (e.g., using cell-free transcription and/or translation systems).

### Fermenting

Suitable conditions for producing the recombinant protein are typically those under which the recombinant host cell according to any of the above can grow and/or remain viable, and produce the recombinant protein.

Non-limiting examples of suitable conditions include a suitable culture medium (e.g., a culture medium having a suitable nutrient content [e.g., a suitable carbon content, a suitable nitrogen content, a suitable phosphorus content], a suitable supplement content, a suitable trace metal content, a suitable pH), a suitable temperature, a suitable feed rate, a suitable pressure, a suitable level of oxygenation, a suitable fermentation duration (i.e., volume of culture media comprising the recombinant host cells), a suitable fermentation volume (i.e., volume of culture media comprising the recombinant host cells), and a suitable fermentation vessel.

Suitable culture media include all culture media in which the recombinant host cell can grow and/or remain viable, and produce the recombinant protein. Typically, the culture medium is an aqueous medium that comprises a carbon source, an assimilable nitrogen source (i.e., a nitrogen-containing compound capable of releasing nitrogen in a form suitable for metabolic utilization by the recombinant host cell), and a phosphate source.

Non-limiting examples of carbon sources include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof.

Non-limiting examples of assimilable nitrogen sources include anhydrous ammonia, ammonium sulfate, ammonium hydroxide, ammonium nitrate, diammonium phosphate, monoammonium phosphate, ammonium pyrophosphate, ammonium chloride, sodium nitrate, urea, peptone, protein hydrolysates, corn steep liquor, corn steep solids, spent grain, spent grain extract, and yeast extract. Use of ammonia gas is convenient for large scale operations, and may be employed by bubbling through the aqueous ferment (fermentation medium) in suitable amounts. At the same time, such ammonia may also be employed to assist in pH control.

The culture medium may further comprise an inorganic salt, a mineral (e.g., magnesium, calcium, potassium, sodium; e.g., in suitable soluble assimilable ionic and combined forms), a metal or transition metal (e.g., copper, manganese, molybdenum, zinc, iron, boron, iodine; e.g., in suitable soluble assimilable form), a vitamin, and any other nutrient or functional ingredient (e.g., a protease [e.g., a plant-based protease] that can prevent degradation of the recombinant protein, a protease inhibitor that can reduce the activity of a protease that can degrade the recombinant protein, and/or a sacrificial protein that can siphon away protease activity, an anti-foaming agent, an anti-microbial agent, a surfactant, an emulsifying oil).

Suitable culture media are available from commercial suppliers or may be prepared according to published compositions (e.g., in catalogues of the American Type Culture Collection).

A suitable pH may be a pH of between about 2 and about 8 (e.g., a pH of between 2 and 8, 7.5, 7, 6.5, 6, 5.5, 5.4, 5.3, 5.2, 5.1, 5, 4.9, 4.8, 4.7, 4.6, 4.5, 4, 3.5, 3, or 2.5; between 2.5 and 8, 7.5, 7, 6.5, 6, 5.5, 5.4, 5.3, 5.2, 5.1, 5, 4.9, 4.8, 4.7, 4.6, 4.5, 4, 3.5, or 3; between 3 and 8, 7.5, 7, 6.5, 6, 5.5, 5.4, 5.3, 5.2, 5.1, 5, 4.9, 4.8, 4.7, 4.6, 4.5, 4, or 3.5; between 3.5 and 8, 7.5, 7, 6.5, 6, 5.5, 5.4, 5.3, 5.2, 5.1, 5, 4.9, 4.8, 4.7, 4.6, 4.5, or 4; between 4 and 8, 7.5, 7, 6.5, 6, 5.5, 5.4, 5.3, 5.2, 5.1, 5, 4.9, 4.8, 4.7, 4.6, or 4.5; between 4.5 and 8, 7.5, 7, 6.5, 6, 5.5, 5.4, 5.3, 5.2, 5.1, 5, 4.9, 4.8, 4.7, or 4.6; between 4.6 and 8, 7.5, 7, 6.5, 6, 5.5, 5.4, 5.3, 5.2, 5.1, 5, 4.9, 4.8, or 4.7; between 4.7 and 8, 7.5, 7, 6.5, 6, 5.5, 5.4, 5.3, 5.2, 5.1, 5, 4.9, or 4.8; between 4.8 and 8, 7.5, 7, 6.5, 6, 5.5, 5.4, 5.3, 5.2, 5.1, 5, or 4.9; between 4.9 and 8, 7.5, 7, 6.5, 6, 5.5, 5.4, 5.3, 5.2, 5.1, or 5; between 5 and 8, 7.5, 7, 6.5, 6, 5.5, 5.4, 5.3, 5.2, or 5.1; between 5.1 and 8, 7.5, 7, 6.5, 6, 5.5, 5.4, 5.3, or 5.2; between 5.2 and 8, 7.5, 7, 6.5, 6, 5.5, 5.4, or 5.3; between 5.3 and 8, 7.5, 7, 6.5, 6, 5.5, or 5.4; between 5.4 and 8, 7.5, 7, 6.5, 6, or 5.5; between 5.5 and 8, 7.5, 7, 6.5, or 6; between 6 and 8, 7.5, 7, or 6.5; between 6.5 and 8, 7.5, or 7; between 7 and 8, or 7.5; or between 7.5 and 8).

A suitable temperature may be a temperature of between about 20° C. and about 46° C. (e.g., between 20° C. and 46° C., 44° C., 42° C., 40° C., 38° C., 36° C., 34° C., 32° C., 30° C., 28° C., 26° C., 24° C., or 22° C.; between 22° C. and 46° C., 44° C., 42° C., 40° C., 38° C., 36° C., 34° C., 32° C., 30° C., 28° C., 26° C., or 24° C.; between 24° C. and 46° C., 44° C., 42° C., 40° C., 38° C., 36° C., 34° C., 32° C., 30° C., 28° C., or 26° C.; between 26° C. and 46° C., 44° C., 42° C., 40° C., 38° C., 36° C., 34° C., 32° C., 30° C., or 28° C.; between 28° C. and 46° C., 44° C., 42° C., 40° C., 38° C., 36° C., 34° C., 32° C., or 30° C.; between 30° C. and 46° C., 44° C., 42° C., 40° C., 38° C., 36° C., 34° C., or 32° C.; between 32° C. and 46° C., 44° C., 42° C., 40° C., 38° C., 36° C., or 34° C.; between 36° C. and 46° C., 44° C., 42° C., 40° C., or 38° C.; between 38° C. and 46° C., 44° C., 42° C., or 40° C.; between 40° C. and 46° C., 44° C., or 42° C., between 42° C. and 46° C. or 44° C.; or between 44° C. and 46° C.).

A suitable feed rate may be a feed rate of between about 0.01 g and about 0.2 g glucose equivalent per g dry cell weight (DCW) per hour.

A suitable pressure may be a pressure of between 0 psig and about 50 psig (e.g., between 0 psig and 50 psig, 40 psig, 30 psig, 20 psig, or 10 psig; between 10 psig and 50 psig, 40 psig, 30 psig, or 20 psig; between 20 psig and 50 psig, 40 psig, or 30 psig; between 30 psig and 50 psig, or 40 psig; or between 40 psig and 50 psig).

A suitable oxygenation may be an aeration rate of between about 0.1 volumes of oxygen per liquid volume in the fermentor per minute (vvm) and about 2.1 vvm (e.g., between 0.1 vvm and 2.1 vvm, 1.9 vvm, 1.7 vvm, 1.5 vvm, 1.3 vvm, 1.1 vvm, 0.9 vvm, 0.7 vvm, 0.5 vvm, or 0.3 vvm; between 0.3 vvm and 2.1 vvm, 1.9 vvm, 1.7 vvm, 1.5 vvm, 1.3 vvm, 1.1 vvm, 0.9 vvm, 0.7 vvm, or 0.5 vvm; between 0.5 vvm and 2.1 vvm, 1.9 vvm, 1.7 vvm, 1.5 vvm, 1.3 vvm, 1.1 vvm, 0.9 vvm, or 0.7 vvm; between 0.7 vvm and 2.1 vvm, 1.9 vvm, 1.7 vvm, 1.5 vvm, 1.3 vvm, 1.1 vvm, or 0.9 vvm; between 0.9 vvm and 2.1 vvm, 1.9 vvm, 1.7 vvm, 1.5 vvm, 1.3 vvm, or 1.1 vvm; between 1.1 vvm and 2.1 vvm, 1.9 vvm, 1.7 vvm, 1.5 vvm, or 1.3 vvm; between 1.3 vvm and 2.1 vvm, 1.9 vvm, 1.7 vvm, or 1.5 vvm; between 1.5 vvm and 2.1 vvm, 1.9 vvm, or 1.7 vvm; between 1.7 vvm and 2.1 vvm or 1.9 vvm; or between 1.9 vvm and 2.1 vvm).

A suitable fermentation duration may be a fermentation duration of between about 10 hours and about 500 hours (e.g., between 10 hours and 500 hours, 400 hours, 300 hours, 200 hours, 100 hours, 50 hours, 40 hours, 30 hours, or 20 hours; between 20 hours and 500 hours, 400 hours, 300 hours, 200 hours, 100 hours, 50 hours, 40 hours, or 30 hours; between 30 hours and 500 hours, 400 hours, 300 hours, 200 hours, 100 hours, 50 hours, or 40 hours; between 40 hours and 500 hours, 400 hours, 300 hours, 200 hours, 100 hours, or 50 hours; between 50 hours and 500 hours, 400 hours, 300 hours, 200 hours, or 100 hours; between 100 hours and 500 hours, 400 hours, 300 hours, or 200 hours; between 200 hours and 500 hours, 400 hours, or 300 hours; between 300 hours and 500 hours, or 400 hours; or between 400 hours and 500 hours).

A suitable fermentation volume may be between about 1 L and about 10,000,000 L (e.g., between 1 L and 10,000,000 L, 5,000,000 L, 1,000,000 L, 500,000 L, 100,000 L, 50,000 L, 10,000 L, 5,000 L, 1,000 L, 500 L, 100 L, 50 L, or 10 L; between 10 L and 10,000,000 L, 5,000,000 L, 1,000,000 L, 500,000 L, 100,000 L, 50,000 L, 10,000 L, 5,000 L, 1,000 L, 500 L, 100 L, or 50 L; between 50 L and 10,000,000 L, 5,000,000 L, 1,000,000 L, 500,000 L, 100,000 L, 50,000 L, 10,000 L, 5,000 L, 1,000 L, 500 L, or 100 L; between 100 L and 10,000,000 L, 5,000,000 L, 1,000,000 L, 500,000 L, 100,000 L, 50,000 L, 10,000 L, 5,000 L, 1,000 L, or 500 L; between 500 L and 10,000,000 L, 5,000,000 L, 1,000,000 L, 500,000 L, 100,000 L, 50,000 L, 10,000 L, 5,000 L, or 1,000 L; between 1,000 L and 10,000,000 L, 5,000,000 L, 1,000,000 L, 500,000 L, 100,000 L, 50,000 L, 10,000 L, or 5,000 L; between 5,000 L and 10,000,000 L, 5,000,000 L, 1,000,000 L, 500,000 L, 100,000 L, 50,000 L, or 10,000 L; between 10,000 L and 10,000,000 L, 5,000,000 L, 1,000,000 L, 500,000 L, 100,000 L, or 50,000 L; between 50,000 L and 10,000,000 L, 5,000,000 L, 1,000,000 L, 500,000 L, or 100,000 L; between 100,000 L and 10,000,000 L, 5,000,000 L, 1,000,000 L, or 500,000 L; between 500,000 L and 10,000,000 L, 5,000,000 L, or 1,000,000 L; between 1,000,000 L and 10,000,000 L, or 5,000,000 L; or between 5,000,000 L and 10,000,000 L).

A suitable fermentation vessel may be any fermentation vessel known in the art. Non-limiting examples of suitable fermentation vessels include culture plates, shake flasks, fermentors (e.g., stirred tank fermentors, airlift fermentors, bubble column fermentors, fixed bed bioreactors, laboratory fermentors, industrial fermentors, or any combination thereof), used at any suitable scale (e.g., small-scale, large-scale) and in any process (e.g., solid culture, submerged culture, batch, fed-batch, or continuous-flow).

### Purifying

Methods for purifying a recombinant protein (e.g., from a fermentation broth) to obtain a preparation comprising the recombinant protein are well-known in the art, and may be adapted to purify the recombinant protein according to any of the above.

The recombinant protein according to any of the above may be purified on the basis of its molecular weight, for example, by size exclusion/exchange chromatography, ultrafiltration through membranes, gel permeation chromatography (e.g., preparative disc-gel electrophoresis), or density centrifugation.

The recombinant protein according to any of the above also may be purified on the basis of its surface charge or hydrophobicity/hydrophilicity, for example, by isoelectric precipitation, anion/cation exchange chromatography, isoelectric focusing (IEF), or reverse phase chromatography.

The recombinant protein according to any of the above also may be purified on the basis of its solubility, for example, by ammonium sulfate precipitation, isoelectric precipitation, surfactants, detergents, or solvent extraction.

The recombinant protein according to any of the above also may be purified on the basis of its affinity to another molecule, for example, by affinity chromatography, reactive dyes, or hydroxyapatite. Affinity chromatography may include the use of an antibody having a specific binding affinity for the recombinant protein, or a lectin to bind to a sugar moiety on the recombinant protein, or any other molecule that specifically binds the recombinant protein. The recombinant protein may comprise a purification tag operably linked to its C-terminus, N-terminus, or both to facilitate affinity-based purification of the recombinant protein. Non-limiting examples of suitable purification tags include affinity tags (i.e., peptides or polypeptides that bind to certain agents or matrices), solubilization tags (i.e., peptides or polypeptides that assist in proper folding of proteins and prevent precipitation), chromatography tags (i.e., peptides or polypeptides that alter the chromatographic properties of a protein to afford different resolution across a particular separation techniques), epitope tags (i.e., peptides or polypeptides that are bound by antibodies), fluorescence tags, chromogenic tags, enzyme substrate tags (i.e., peptides or polypeptides that are the substrates for specific enzymatic reactions), chemical substrate tags (i.e., peptides or polypeptides that are the substrates for specific chemical modifications), self-cleaving tags (peptides or polypeptides that possess inducible proteolytic activity; e.g., Sortase tag, Npro tag, FrpC module, CPD), hydrophobic tags (proteins or polypeptides that are highly hydrophobic and direct the protein for inclusion body formation; e.g., KSI tag, TrpE tag), or combinations thereof. Non-limiting examples of suitable affinity tags include maltose binding protein (MBP) tag, glutathione-S-transferase (GST) tag, poly(His) tag, hexa(His) tag, SBP-tag, Strep-tag, and calmodulin-tag. Non-limiting examples of suitable solubility tags include thioredoxin (TRX) tag, poly(NANP) tag, MBP tag, SUMO tag, GB1 tag, NUSA CBD tag, and GST tag. Non-limiting examples of chromatography tags include polyanionic amino acid tags (e.g., FLAG-tag) and polyglutamate tag. Non-limiting examples of epitope tags include V5-tag, VSV-tag, E-tag, NE-tag, hemagglutinin (Ha)-tag, Myc-tag, and FLAG-tag. Non-limiting examples of fluorescence tags include green fluorescent protein (GFP) tag, blue fluorescent protein (BFP) tag, cyan fluorescent protein (CFP) tag, yellow fluorescent protein (YFP) tag, orange fluorescent protein (OFP) tag, red fluorescent protein (RFP) tag, and derivatives thereof. Non-limiting examples of enzyme substrate tags include peptides or polypeptides comprising a lysine within a sequence suitable for biotinilation (e.g., AviTag, Biotin Carboxyl Carrier Protein [BCCP]). Non-limiting examples of chemical substrate tags include substrates suitable for reaction with FIAsH-EDT2. The tag peptide or polypeptide may be removed following isolation of the recombinant protein (e.g., via protease cleavage).

In embodiments in which the recombinant protein according to any of the above is secreted by the recombinant host cell according to any of the above, the recombinant protein may be purified directly from the culture medium. In other embodiments, the recombinant protein may be purified from a cell lysate.

The recombinant protein may be purified to obtain a preparation comprising the recombinant protein at a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% relative to other components comprised in the fermentation broth; or to at least 2-fold, at least 3-fold, at least 4-fold, at least 5-fold, at least 6-fold, at least 7-fold, at least 8-fold, at least 9-fold, or at least 10-fold greater abundancy relative to other components comprised in the fermentation broth; or to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% by mass of total protein.

The identity of the recombinant protein may be confirmed and/or quantified by high performance liquid chromatography (HPLC), Western blot analysis, Eastern blot analysis, polyacrylamide gel electrophoresis, capillary electrophoresis, formation of an enzyme product, disappearance of an enzyme substrate, and 2-dimensional mass spectroscopy (2D-MS/MS) sequence identification.

### Post-Processing

Post-processing may alter certain chemical and/or physical properties of the recombinant protein, including but not limited to size, charge, hydrophobicity, hydrophilicity, solvation, protein folding, and chemical reactivity.

Post-processing may comprise refolding of the recombinant protein (e.g., by removing a denaturant); fragmenting of the recombinant protein (e.g., by chemical means or by exposure to proteases [e.g., trypsin, pepsin]); heating of the recombinant protein (e.g., to remove protein aggregates); removing reactive sites of the recombinant protein (e.g., removing reactive sites of methionine and/or tryptophan residues by oxidation); modulating of the recombinant protein (e.g., via chemical, photochemical, and/or enzymatic strategies); demineralizing of a preparation comprising the recombinant protein (by, e.g., dialysis, ultrafiltration, reverse osmosis, ion exchange chromatography); removing tags and/or fusion polypeptides from the recombinant protein (e.g., by exposure to site-specific proteases); biotinylating of the recombinant protein (i.e., attaching biotin); and/or conjugating of the recombinant protein to other elements (e.g., poly-ethylene-glycol, antibodies, liposomes, phospholipids, DNA, RNA, polynucleotides, sugars, disaccharides, polysaccharides, starches, cellulose, detergents, cell walls).

Post-processing may occur in a random manner or in a site-specific manner (e.g., at sulfhydryl groups of cysteine residues [e.g., for aminoethylation, formation of iodoacetamides, formation of maleimides, formation of Dha, covalent attachment via disulfide bonds, and desulfurization], at primary amine groups of lysine residues [e.g., for attachment of activated esters, sulfonyl chlorides, isothiocyanates, unsaturated aldehyde esters, and aldehydes], at phenolic hydroxyl groups of tyrosine residues, at specific allergenic epitopes [e.g., glycan groups]).

The recombinant protein may be dried (e.g., via spray drying or lyophilization) or concentrated (e.g., via precipitation or evaporation) (e.g., to obtain a powder).

### Method for Producing Casein

In various aspects, provided herein is a method for producing a casein (e.g., K-casein, β-casein, α-S1-casein, α-S2-casein), wherein the method comprises fermenting a recombinant host cell according to any of the above; wherein the recombinant host cell is capable of producing a recombinant protein having a structure (A), (B), or (C) according to any of the above; wherein at least one of the milk protein 1, the milk protein 2, the milk protein 3, and the milk protein 4 of the recombinant protein is a β-lactoglobulin; wherein at least one of the milk protein 1, the milk protein 2, the milk protein 3, and the milk protein 4 of the recombinant protein is the casein; and wherein the casein is adjacent to at least one linker peptide comprising a recognition or cleavage sequence for a protease (e.g., any of the protease recognition or cleavage sequences disclosed herein).

Without wishing to be limited by theory, it is believed that covalent linkage of the casein to the β-lactoglobulin may prevent aggregation of the casein and thereby provide for higher titer production of recombinant casein by the recombinant host cell.

Non-limiting examples of recombinant proteins suitable for use in the method according to the above include any of the following:
a structure (A) according to the above having the form: (β-lactoglobulin)-(linker peptide 1)-(casein),
a structure (B) according to the above having the form: (β-lactoglobulin)-(linker peptide 1)-(casein)-(linker peptide 2)-(β-lactoglobulin),
a structure (B) according to the above having the form: (β-lactoglobulin)-(linker peptide 1)-(casein 1)-(linker peptide 2)-(casein 2),
a structure (C) according to the above having the form: (β-lactoglobulin)-(linker peptide 1)-(casein 1)-(linker peptide 2)-(casein 2)-(linker peptide 3)-(β-lactoglobulin),
a structure (C) according to the above having the form: (β-lactoglobulin)-(linker peptide 1)-(casein 1)-(linker peptide 2)-(β-lactoglobulin)-(linker peptide 3)-(casein 2), and
a structure (C) according to the above having the form: (β-lactoglobulin)-(linker peptide 1)-(casein 1)-(linker peptide 2)-(casein 2)-(linker peptide 3)-(casein 3);
   wherein
linker peptide 1, the linker peptide 2, and the linker peptide 3 may comprise a recognition or cleavage sequence for a protease (e.g., any of the protease recognition or cleavage sequences disclosed herein),
the linker peptide 1, the linker peptide 2, and the linker peptide 3 may be identical to each other or differ from each other in length and/or amino acid sequence, and
the casein 1, the casein 2, and the casein 3 may be identical to each other or differ from each other in length and/or amino acid sequence.

The method may further comprise cleaving the linker peptide with the protease, and thereby releasing the casein.

### Polymer Comprising Recombinant Protein

In various aspects, provided herein is a polymer.

The polymer may include a protein polymer, wherein the protein polymer comprises protein monomers linked via disulfide bonds. The protein monomers may be one or more recombinant proteins according to any of the above. For example, the protein monomers may be a single recombinant protein. Alternatively, the protein monomers may be at least two different recombinant proteins.

The polymer provides advantages in production, use, and disposal, including but not limited to smaller negative impact on the environment due to the use of renewable natural resources for its production and the potential for biodegradability, as well as petroleum resource independence. Moreover, the polymer may comprise monomers derived from a subset of milk proteins (i.e., the polymer is essentially free of other milk proteins not comprised in the recombinant protein, and of impurities comprised in mammalian-derived milk). Moreover, the polymer may be produced from purified preparations of recombinant protein, providing greater control over polymerization processes and tailoring of polymer characteristics. Specifically, in contrast to producing polymers from mammal-derived milk comprising a mixture of various proteins, lipids, and other components that can impact process and outcome of a polymerization process (e.g., affect stability and/or other attributes of a polymer), production of a polymer from the recombinant protein according to any of the above constitutes a limited-component system that may provide better control over process and outcome.

The polymer may comprise between about 0.001% and about 100% (e.g., between 0.001% and 100%, 99%, 95%, 90%, 80%, 70%, 60%, 50%, 40%, 30%, 20%, 10%, 1%, 0.1%, or 0.01%; between 0.01% and 100%, 99%, 95%, 90%, 80%, 70%, 60%, 50%, 40%, 30%, 20%, 10%, 1%, or 0.1%; between 0.1% and 100%, 99%, 95%, 90%, 80%, 70%, 60%, 50%, 40%, 30%, 20%, 10%, or 1%; between 1% and 100%, 99%, 95%, 90%, 80%, 70%, 60%, 50%, 40%, 30%, 20%, or 10%; between 10% and 100%, 99%, 95%, 90%, 80%, 70%, 60%, 50%, 40%, 30%, or 20%; between 20% and 100%, 99%, 95%, 90%, 80%, 70%, 60%, 50%, 40%, or 30%; between 30% and 100%, 99%, 95%, 90%, 80%, 70%, 60%, 50%, or 40%; between 40% and 100%, 99%, 95%, 90%, 80%, 70%, 60%, or 50%; between 50% and 100%, 99%, 95%, 90%, 80%, 70%, or 60%; between 60% and 100%, 99%, 95%, 90%, 80%, or 70%; between 70% and 100%, 99%, 95%, 90%, or 80%; between 80% and 100%, 99%, 95%, or 90%; between 90% and 100%, 99%, or 95%; between 95% and 100% or 99%; or between 99% and 100%) by mass of the protein polymer.

The protein polymer may comprise between about 0.001% and about 100% (e.g., between 0.001% and 100%, 99%, 95%, 90%, 80%, 70%, 60%, 50%, 40%, 30%, 20%, 10%, 1%, 0.1%, or 0.01%; between 0.01% and 100%, 99%, 95%, 90%, 80%, 70%, 60%, 50%, 40%, 30%, 20%, 10%, 1%, or 0.1%; between 0.1% and 100%, 99%, 95%, 90%, 80%, 70%, 60%, 50%, 40%, 30%, 20%, 10%, or 1%; between 1% and 100%, 99%, 95%, 90%, 80%, 70%, 60%, 50%, 40%, 30%, 20%, or 10%; between 10% and 100%, 99%, 9%, 90%, 80%, 70%, 60%, 50%, 40%, 30%, or 20%; between 20% and 100%, 99%, 95%, 90%, 80%, 70%, 60%, 50%, 40%, or 30%; between 30% and 100%, 99%, 95%, 90%, 80%, 70%, 60%, 50%, or 40%; between 40% and 100%, 99%, 95%, 90%, 80%, 70%, 60%, or 50%; between 50% and 100%, 99%, 95%, 90%, 80%, 70%, or 60%; between 60% and 100%, 99%, 95%, 90%, 80%, or 70%; between 70% and 100%, 99%, 95%, 90%, or 80%; between 80% and 100%, 99%, 95%, or 90%; between 90% and 100%, 99%, or 95%; between 95% and 100% or 99%; or between 99% and 100%) by mass of the one or more recombinant proteins.

The repeated protein monomers may further be linked via intermediary molecules, and/or via other covalent bonds (e.g., amide bonds [e.g., lactam bridges, native chemical ligation bonds, Staudinger ligation bonds]) and/or via non-covalent interactions (e.g., electrostatic interactions, hydrogen bonds, steric interactions, Van der Waals interactions).

The polymer according to any of the above may be a polymer blend that is produced by mixing the recombinant protein according to any of the above with one or more other components capable of interacting with the recombinant protein (e.g., petroleum-derived compounds [e.g., ethylene, propylene, vinyl chloride], carbohydrates [e.g., alginate, chitosan, cellulose], non-milk proteins [e.g., plant proteins (e.g., any of the plant proteins disclosed herein), animal proteins (e.g., collagen, gelatin, silk fibroin, keratin, ovalalbumin), fungal proteins (e.g., any of the fungal proteins disclosed herein), and combinations thereof]). The mass ratio of the recombinant protein to the one or more other components may be between about 1 to 100 and about 100 to 1 (e.g., about 100 to 1, about 90 to 1, about 80 to 1, about 70 to 1, about 60 to 1, about 50 to 1, about 40 to 1, about 30 to 1, about 20 to 1, about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10, about 1 to 20, about 1 to 30, about 1 to 40, about 1 to 50, about 1 to 60, about 1 to 70, about 1 to 80, about 1 to 90, or about 1 to 100).

The polymer according to any of the above may resemble a conventional petroleum-derived polymer. Such resemblance may be due to any one or any combination of two or more of the attributes disclosed herein.

The polymer according to any of the above may be essentially free of any protein other than the recombinant protein or the recombinant proteins contained in the polymer according to any of the above.

The polymer according to any of the above may be essentially free of any recombinant protein other than the recombinant protein or the recombinant proteins contained in the polymer according to any of the above.

The polymer according to any of the above may be essentially free of any recombinant milk protein other than the recombinant protein or the recombinant proteins contained in the polymer according to any of the above.

The polymer according to any of the above may be essentially free of a component found in a mammal-produced milk (e.g., cow milk, goat milk, sheep milk, human milk, buffalo milk, yak milk, camel milk, llama milk, alpaca milk, horse milk, donkey milk), or may comprise a lower concentration of at least one component found in a mammal-produced milk. Non-limiting examples of components found in mammal-derived milk include lactose, saturated fat, cholesterol, native milk proteins, and native milk lipids. The polymer may be essentially free of any milk protein other than the milk protein comprised in the recombinant protein according to any of the above.

The polymer according to any of the above may be essentially free of a component obtained from an animal (i.e., a component that is native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk, milk lipid extracted from mammal-derived milk, animal lipids, animal proteins]), or comprise 2% or less by mass of such component.

The polymer according to any of the above may be essentially free of a component derived from petroleum, or comprise 2% or less by mass of a component derived from petroleum

### Method for Producing Polymer

In various aspects, provided herein is a method for producing a polymer according to any of the above, wherein the method comprises:
a) obtaining one or more recombinant proteins according to any of the above (e.g., via production by a recombinant host cell according to any of the above), and
b) mildly denaturing the one or more recombinant proteins such that one or more free thiol groups comprised in the one or more recombinant proteins form one or more intra- and/or inter-molecular disulfide bonds.

Mildly denaturing the recombinant protein (e.g., denaturing less than 20% of the recombinant protein) can be accomplished under conditions that expose free thiol group(s) comprised in the recombinant protein but do not break existing intra-molecular disulfide bonds in the recombinant protein. Such conditions can include heating the recombinant protein to a temperature (e.g., 10-20° C. below the Tm of the recombinant protein) at a pH at which the conformation of the recombinant protein is relatively stable (i.e., a pH at which less than 10% of the recombinant protein is denatured), or heating the recombinant protein to a temperature (e.g., 15-25° C. below the Tm of the recombinant protein) at a pH at which the conformation of the recombinant protein is slightly unstable (i.e., a pH at which between 10% and 50% of the recombinant protein is denatured), or heating the recombinant protein to a temperature (e.g., 20-30° C. below the Tm of the recombinant protein) at a pH at which the conformation of the recombinant protein is unstable (i.e., a pH at which more than 50% of the recombinant protein is denatured). The mildly denaturing condition can also be modulated by the duration of the heating.

Disulfide bond formation may occur under non-reducing conditions at a suitable pH and suitable ionic strength, at a suitable temperature, and at a suitable heating time (see, for example, Ramos et al. 2017 Crit Rev Food Sci Nutr. 57(7):1377-1393, Tavares et al. 2014 Trends in Food Science and Technology 37:5-20, Teng et al. 2015 RSC Adv. 5:35138, Bourbon et al. 2019 Gels 5:9, Ahmed et al. 2016 Colloids and Surfaces B: Biointerfaces 143:371-381, Remondetto and Subirade 2003 Biopolymers 69:461-469, Gunasekaran et al. 2007 Journal of Food Engineering 83:31-40). Non-limiting examples of suitable temperatures include temperatures of between about 0° C. and about 30° C. below the Tm of the recombinant protein (e.g., temperature of between 35° C. and 105° C., 100° C., 95° C., 90° C., 85° C., 80° C., 75° C., 70° C., 65° C., 60° C., 55° C., 50° C., 45° C., or 40° C.; between 40° C. and 105° C., 100° C., 95° C., 90° C., 85° C., 80° C., 75° C., 70° C., 65° C., 60° C., 55° C., 50° C., or 45° C.; between 45° C. and 105° C., 100° C., 95° C., 90° C., 85° C., 80° C., 75° C., 70° C., 65° C., 60° C., 55° C., or 50° C.; between 50° C. and 105° C., 100° C., 95° C., 90° C., 85° C., 80° C., 75° C., 70° C., 65° C., 60° C., or 55° C.; between 55° C. and 105° C., 100° C., 95° C., 90° C., 85° C., 80° C., 75° C., 70° C., 65° C., or 60° C.; between 60° C. and 105° C., 100° C., 95° C., 90° C., 85° C., 80° C., 75° C., 70° C., or 65° C.; between 65° C. and 105° C., 100° C., 95° C., 90° C., 85° C., 80° C., 75° C., or 70° C.; between 70° C. and 105° C., 100° C., 95° C., 90° C., 85° C., 80° C., or 75° C.; between 75° C. and 105° C., 100° C., 95° C., 90° C., 85° C., or 80° C.; between 80° C. and 105° C., 100° C., 95° C., 90° C., or 85° C.; between 85° C. and 105° C., 100° C., 95° C., or 90° C.; between 90° C. and 105° C., 100° C., or 95° C.; between 95° C. and 105° C. or 100° C.; or between 100° C. and 105° C.). Non-limiting examples of suitable pHs include pHs of less than 11, less than 10.5, less than 10, less than 9.5, less than 9, less than 8.5, less than 8, less than 7.5, less than 7, less than 6.5, less than 6, less than 5.5, less than 5, less than 4.5, or less than 4; or between a out 3.5 and about 11 (e.g., pH of between 3.5 and 11, 10.5, 10, 9.5, 9, 8.5, 8, 7.5, 7, 6.5, 6, 5.5, 5, 4.5, or 4; between 4 and 11, 10.5, 10, 9.5, 9, 8.5, 8, 7.5, 7, 6.5, 6, 5.5, 5, or 4.5; between 4.5 and 11, 10.5, 10, 9.5, 9, 8.5, 8, 7.5, 7, 6.5, 6, 5.5, or 5; between 5 and 11, 10.5, 10, 9.5, 9, 8.5, 8, 7.5, 7, 6.5, 6, or 5.5; between 5.5 and 11, 10.5, 10, 9.5, 9, 8.5, 8, 7.5, 7, 6.5, or 6; between 6 and 11, 10.5, 10, 9.5, 9, 8.5, 8, 7.5, 7, or 6.5; between 6.5 and 11, 10.5, 10, 9.5, 9, 8.5, 8, 7.5, or 7; between 7 and 11, 10.5, 10, 9.5, 9, 8.5, 8, or 7.5; between 7.5 and 11, 10.5, 10, 9.5, 9, 8.5, or 8; between 8 and 11, 10.5, 10, 9.5, 9, or 8.5; between 8.5 and 11, 10.5, 10, 9.5, or 9; between 9 and 11, 10.5, 10, or 9.5; between 9.5 and 11, 10.5, or 10; between 10 and 11, or 10.5; or between 10.5 and 11). Non-limiting examples of suitable heating times include heating times of between 5 min and 120 min, 110 min, 100 min, 90 min, 80 min, 70 min, 60 min, 50 min, 40 min, 30 min, 20 min, or 10 min; or between 10 min and 120 min, 110 min, 100 min, 90 min, 80 min, 70 min, 60 min, 50 min, 40 min, 30 min, or 20 min; between 20 min and 120 min, 110 min, 100 min, 90 min, 80 min, 70 min, 60 min, 50 min, 40 min, or 30 min; between 30 min and 120 min, 110 min, 100 min, 90 min, 80 min, 70 min, 60 min, 50 min, or 40 min; between 40 min and 120 min, 110 min, 100 min, 90 min, 80 min, 70 min, 60 min, or 50 min; between 50 min and 120 min, 110 min, 100 min, 90 min, 80 min, 70 min, or 60 min; between 60 min and 120 min, 110 min, 100 min, 90 min, 80 min, or 70 min; between 70 min and 120 min, 110 min, 100 min, 90 min, or 80 min; between 80 min and 120 min, 110 min, 100 min, or 90 min; between 90 min and 120 min, 110 min, or 100 min; between 100 min and 120 min, or 110 min; or between 110 min and 120 min.

In addition, polymerization may be modified using a variety of other methods for polymerizing protein monomers that are known in the art, such as, for example, extrusion of the recombinant protein (see, for example, Tavares et al. 2014 Trends in Food Science and Technology 37:5-20, Teng et al. 2015 RSC Adv. 5, 35138); encapsulating of active agents (see, for example, Tavares et al. 2014 Trends in Food Science and Technology 37:5-20); electrospinning (see, for example, Stie et al. 2020 ACS Appl. Nano Mater. 3:1910-1921, Sullivan, et al. 2014 Food Hydrocolloids 35:36-50, Akhmetova & Heinz 2021 Pharmaceutics 13:4, Ahmed et al. 2016 Colloids and Surfaces B: Biointerfaces 143: 371-381); processing to hydrogels, hydronanogels, nanofibrils, nanoparticles, nanotubes, nanogels, hydrogel beads, biopolymer particles, or microspheres (see, for example, Ramos et al. 2017 Crit Rev Food Sci Nutr. 57(7):1377-1393, Gunasekaran et al. 2007 Journal of Food Engineering 83: 31-40, McClements 2017 Advances in Colloid and Interface Science 240: 31-59), and processing to films and/or coatings (e.g., for producing edible films and coatings, and/or for delivering encapsulated nanoparticles, nanoemulsions, and gels; (see, for example, Ramos et al. 2012 Critical Reviews in Food Science and Nutrition, 52(6):533-552), processing to foams (see, for example, Ye et al. 2021 Adv. Sustainable Syst. 2100063), methods that employ crosslinking agents (i.e., chemicals that activate functional groups on proteins and thus connect proteins without incorporating a spacer), crosslinking enzymes (e.g., transferases [enzyme commission number (EC) 2; e.g., transglutaminases], hydrolases (EC 3)), oxidation (e.g., using oxidizing agents), radiation (e.g., using UV, gamma, electron beam), mechanical agitation, electrical field, pressure (e.g., extrusion), turbulence, friction, photo-oxidative treatment (e.g., using photo-reactive amino acid analogs), 3D-printing, electrospinning, and combinations thereof.

As would be familiar to the skilled person, the components can be blended in various ways, for example, dry blending or melt blending the components. For example, a polyethylene resin and protein polymer may be dry blended, for example, by adding the components as pellets in an extruder, wherein the components are heated and mixed together. Alternatively, the polyethylene resin and protein polymer may be melt blended, wherein each component is melted and mixed in a compounder or extruder and then pelletized. Other blending methodologies for mixing the components are contemplated herein.

### Composition Comprising Recombinant Protein

In various aspects, provided herein is a composition that comprises or consists essentially of the recombinant protein according to any of the above and/or the polymer according to any of the above.

The composition may comprise between about 0.1% and about 100% (e.g., between 0.1% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9% 8%, 7%, 6%, 5%, 4%, 3%, 2%, 1%, 0.9%, 0.8%, 0.7%, 0.6%, 0.5%, 0.4%, or 0.3%; between 0.3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 66%, 5%, 4%, 3%, 2%, 1%, 0.9%, 0.8%, 0.7%, 0.6%, 0.5%, or 0.4%; between 0.4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, or 0.8%; between 0.8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 100%, 95%, 90%, 85%, 80%, 75%, 70%, 65%, 60%, 55%, 50%, 45%, 440%, 35%, 30%, 25%, 20%, 15%, 14%, 13%, 12%, 11%, 10%, 9%, 8%, 7%, 6%, 5%, 4%, 3%, or 2%; between 2% and 100%, 95%, 90%, 85%, 80%, 75%, 70%, 65%, 60%, 55%, 50%, 45%, 440%, 35%, 330%, 25%, 20%, 15%, 14%, 13%, 12%, 11%, 10%, 9%, 8%, 7%, 6%, 5%, 4%, or 3%; between 3% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 100%, 95%, 90%, 85%, 80%, 75%, 70%, 65%, 60%, 55%, 50%, 45%, 440%, 35%, 330%, 25%, 20%, 15%, 14%, 13%, 12%, 11%, 10%, 9%, 8%, 7%, or 6%; between 6% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 100%, 95%, 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 100%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 100%, 95%, 90%, 85%, 80%, 75%, 70%, 65%, 60%, 55%, 50%, 45%, 40%, 35%, 30%, 25%, 20%, 15%, 14%, 13%, or 12%; between 12% and 100%, 95%, 90%, 85%, 80%, 75%, 70%, 65%, 60%, 55%, 50%, 45%, 40%, 35%, 30%, 25%, 20%, 15%, 14%, or 13%; between 13% and 100%, 95%, 90%, 85%, 80%, 75%, 70%, 65%, 60%, 55%, 50%, 45%, 40%, 35%, 30%, 25%, 20%, 15%, or 14%; between 14% and 100%, 95%, 90%, 85%, 80%, 75%, 70%, 65%, 60%, 55%, 50%, 45%, 40%, 35%, 30%, 25%, 20%, or 15%; between 15% and 100%, 95%, 90%, 85%, 80%, 75%, 70%, 65%, 60%, 55%, 50%, 45%, 40%, 35%, 30%, 25%, or 20%; between 20% and 100%, 95%, 90%, 85%, 80%, 75%, 70%, 65%, 60%, 55%, 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 100%, 95%, 90%, 85%, 80%, 75%, 70%, 65%, 60%, 55%, 50%, 45%, 40%, or 35%; between 35% and 100%, 95%, 90%, 85%, 80%, 75%, 70%, 65%, 60%, 55%, 50%, 45%, or 40%; between 40% and 100%, 95%, 90%, 85%, 80%, 75%, 70%, 65%, 60%, 55%, 50%, or 45%; between 45% and 100%, 95%, 90%, 85%, 80%, 75%, 70%, 65%, 60%, 55%, or 50%; between 50% and 100%, 95%, 90%, 85%, 80%, 75%, 70%, 65%, 60%, or 55%; between 55% and 100%, 95%, 90%, 85%, 80%, 75%, 70%, 65%, or 60%; between 60% and 100%, 95%, 90%, 85%, 80%, 75%, 70%, or 65%; between 65% and 100%, 95%, 90%, 85%, 80%, 75%, or 70%; between 70% and 100%, 95%, 90%, 85%, 80%, or 75%; between 75% and 100%, 95%, 90%, 85%, or 80%; between 80% and 100%, 95%, 90%, or 85%; or between 85% and 100%, 95%, 90%; between 90% and 100% or 95%, or between 95% and 100%) by dry mass of the recombinant protein and/or polymer.

At standard ambient temperature and conditions (i.e., 20-30° C. and 0.95-1.05 atm), the composition according to any of the above may be a fluid, semi-solid (e.g., gelatinous), solid, or powder. The powder may comprise a moisture content of less than 20%, less than 15%, less than 10%, less than 7%, less than 5%, less than 3%, or less than 1%; or between about 0.1% and about 20% (e.g., between 0.1% and 20%, 15%, 10%, 5%, or 1%; between 1% and 20%, 15%, 10%, or 5%; between 5% and 20%, 15%, or 10%; between 10% and 20%, or 15%; or between 15% and 20%). The powder may be used in powder form, or the powder may be reconstituted with a hydrating agent prior to use, or the powder may be mixed with other dry components (e.g., flour, sugar, minerals, pH or ionic strength adjusting agents) before a hydrating agent is added to the mixture.

The composition according to any of the above may further comprise an other ingredient (e.g., any of the other ingredients disclosed herein). For example, the composition may comprise between about 0.001% and about 50% (e.g., between 0.001% and 50%, 25%, 20%, 15%, 10%, 9%, 8%, 7%, 6%, 5%, 4%, 33%, 2%, 1%, 0.5%, 0.1%, or 0.01%; between 0.01% and 50%, 25%, 20%, 15%, 10%, 9%, 8%, 70%, 6%, 5%, 4%, 3%, 2%, 1%, 0.5%, or 0.1%; between 0.1% and 50%, 25%, 20%, 15%, 10%, 9%, 8%, 7%, 6%, 5%, 4%, 3%, 2%, 1%, or 0.5%; between 0.5% and 50%, 25%, 20%, 15%, 10%, 9%, 8%, 7%, 6%, 5%, 4%, 3%, 2%, or 1%; between 1% and 50%, 25%, 20%, 15%, 10%, 9%, 8%, 7%, 6%, 5%, 4%, 3%, or 2%; between 2% and 50%, 25%, 20%, 15%, 10%, 9%, 8%, 7%, 6%, 5%, 44%, or 3%; between 3% and 50%, 25%, 20%, 15%, 10%, 9%, 8%, 7%, 6%, 5%, or 4%; between 4% and 50%, 25%, 20%, 15%, 10%, 9%, 8%, 7%, 6%, or 5%; between 5% and 50%, 25%, 20%, 15%, 10%, 9%, 8%, 7%, or 6%; between 6% and 50%, 25%, 20%, 15%, 10%, 9%, 8%, or 7%; between 7% and 50%, 25%, 20%, 15%, 10%, 9%, or 8%; between 8% and 50%, 25%, 20%, 15%, 10%, or 9%; between 9% and 50%, 25%, 20%, 15%, or 10%; between 10% and 50%, 25%, 20%, or 15%; between 15% and 50%, 25%, or 20%; between 20% and 50% or 25%; or between 25% and 50%) by mass of any one such other ingredient, any combination of two or more such other ingredients, or all such other ingredients.

The composition according to any of the above may be a variety of products, including, for example, an encapsulate (e.g., a product that encapsulates a therapeutic or nutraceutical for delivery [e.g., a micro- or nano-particle (e.g., a bead, a micelle, a capsule, a hydrogel), a composition with industrial utility (e.g., a dielectric), an adhesive (i.e., a material that forms an adhesive bond; e.g., glue, wallpaper adhesive, wood adhesive, paper adhesive, cork adhesive, chipboard adhesive, surgical/medical glue, cement, mucilage, paste), a coating or facing (e.g., glossy coating, protective coating, varnish, coating for medical tablet, paper coating, painting, leather finishing, textile coating), a spray, a paint or ink or pigment binder for ink, a hard plastic (e.g., bottle, button, window, pen), a medium hard plastic (e.g., bottle, fiber [e.g., yarn]), a soft plastic (e.g., bag, wrap, edible film, waterproof film, contact lens, packaging material), a fabric (e.g., textile, carpet, curtain, clothing), an industrial polymer (i.e., compound used in the manufacture of synthetic industrial materials), a medical diagnostic (see, for example, J. Berger et al. 2004. Europ J of Pharm and Biopharm 57:19, respectively), a gel (e.g., hydrogel for controlled release of a therapeutic, hydrogel for immobilizing a protein (e.g., enzyme)), an implant (e.g., bone-replacing composite, material supporting nerve repair, scaffold for growing cells, prosthetic implant), an article of clothing (e.g., shoe), a lubricant, a piece of furniture, a paper (e.g., paper sheet, paper label, packaging paper, photographic support), a household item (e.g., pot, bowl, plate, cup), and a biological scaffold (i.e., a structure that mimics a biological matrix, sutures, bone-replacing material, material supporting nerve repair, scaffold for growing cells, prosthetic implant, membrane for promoting wound healing, wound dressing, tissue-engineering scaffolding).

### Other Ingredients

The other ingredient optionally comprised in the composition according to any of the above may be any other ingredient.

Non-limiting examples of suitable other ingredients include other proteins, bioactive agents, nutritional agents, and functional agents.

The optional other proteins may consist essentially of one or more native and/or recombinant other proteins derived from any source, as well as mixtures of native and/or recombinant other proteins derived from various sources. Non-limiting examples of suitable sources include animals, plants, algae, fungi, or bacteria. Non-limiting examples of animal proteins include structural animal proteins (e.g., collagen, tropoelastin, elastin, keratin, silk fibroin; see, for example, Akhmetova & Heinz, 2021 Pharmaceutics 13:4), milk proteins (e.g., any of the whey proteins or caseins disclosed herein in non-polymerized form), egg proteins (e.g., hereinovomucoid, ovalbumin, ovotransferrin, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y,), silk fibroin, growth factors (e.g., vascular endothelial growth factor), bioactive peptides, therapeutic peptides (e.g., antimicrobial peptides [e.g., magainins, cecropins], pore-forming peptides that are derived from the Bel-2 family of apoptosis mediators; see, for example, Boohaker et al. 2012 Curr Med Chem. 19(22):3794-804), and globular proteins (e.g., albumin). Non-limiting examples of plant proteins include pea proteins (e.g., legumin, vicillin, covicillin), soybean proteins, potato proteins (e.g., tuberin, protease inhibitor notate II), wheat protein, and zein. Non-limiting examples of fungal proteins include glucoamylase, xylanase, amylase, glucanase, member of the SUN family (Sim1p, Uth1p, Nca3p, Sun4p), elongation factor 1-alpha, mitochondrial leucyl-tRNA synthetase, alpha-amylase, alpha-galactosidase, cellulase, endo-1,4-beta-xylanase, endoglucanase, exo-1,4-beta-xylosidase, glucoamylase, peptidase, aspergillopepsin-1, 1,4-beta-D-glucan cellobiohydrolase A, alpha-galactosidase A, alpha-galactosidase B, alpha-galactosidase D, alpha-glucuronidase A, beta-galactosidase C, glucan 1,3-beta-glucosidase A, hydrophobin, and glucan endo-1,3-beta-glucosidase eglC. The optional other proteins may comprise a recombinant other protein (e.g., a recombinant other protein having a mammalian PTM, a non-mammalian PTM, or a mixture thereof, and/or lacking a mammalian PTM, and/or lacking an epitope that can elicit an immune response in a human or animal).

Non-limiting examples of bioactive agents include neutraceuticals (i.e., compounds that have physiological benefit or provide protection against disease), and therapeutics (i.e., compounds that treat disease).

Non-limiting examples of nutritional agents include nutritional supplements, prebiotics, probiotics, pro-vitamins, vitamins, minerals, antioxidants, carbohydrates, lipids, and essential and semi-essential amino acids.

Non-limiting examples of vitamins include lipid soluble vitamins, water soluble vitamins, thiamin (vitamin B1), riboflavin (vitamin B2), niacin (vitamin B3), pantothenic acid (vitamin B5, respectively), pyridoxine (vitamin B6), folic acid (vitamin B9), cobalamin (vitamin B12), vitamin C, folate, vitamin A, vitamin D2, vitamin D3, vitamin D, vitamin E, vitamin K, and derivatives and mixtures thereof.

Non-limiting examples of minerals include calcium, phosphorous, potassium, sodium, citrate, chloride, phosphate, sulfate, magnesium, potassium, zinc, iron, molybdenum, manganese, copper, and mixtures thereof.

Non-limiting examples of antioxidants include α-tocopherol (e.g., tocopherol comprised in Bos taurus milk), low molecular weight thiols (e.g., low molecular weight thiols comprised in Bos taurus milk), retinol (e.g., retinol comprised in Bos taurus milk), carotenoids (e.g., carotenoids comprised in cow milk, α-carotene, β-carotene, γ-carotene, lutein, zeaxanthin, astaxanthin), vitamin E, Azadirachta indica extract, riboflavin, rosemary extract, phenolic diterpenes (e.g., carnosol, carnosic acid) comprised in rosemary extract, sage extract, ascorbic acid (vitamin C) and its salts, lactic acid and its salts, grape residue silage, phenolic compounds (e.g., ferulic acid) comprised in grape residue silage, soybean (Glycine max) extract, isoflavones or polyphenolic compounds comprised in soybean extract, garlic (Allium sativum) extract, phenolic or flavonoid, or terpenoid compounds comprised in garlic extract, fennel (Foeniculum vulgare Mill.) extract, chamomile (Matricaria recutita L.) extract, fatty acids (e.g., alpha-lipoic acid), brown algae (e.g., Ascophyllum nodosum, Fucus vesiculosus), essential oils of green pink pepper (GEO), essential oils of mature pink pepper (MEO), green tea extract, butylated hydroxyanisole (E320), butylated hydroxytoluene (E321), polyphenols (e.g., curcumins, curcuminoids, desmethoxycurcumin (hydroxycirmamoyl feruloylmethane), bis-desmethoxycurcumin), catechins (e.g., epigallocatechin gallate, epicatechin gallate, epigallocatechin, epicatechin, C catechin, catechins comprised in green tea extract), and derivatives and mixtures thereof.

Non-limiting examples of carbohydrates include: monosaccharides, such as, for example, glucose, fructose, galactose, and mixtures thereof; disaccharides, such as, for example, maltose, lactose, sucrose, and mixtures thereof; polysaccharides, such as for example, starches (e.g., pectin, corn (maize) starch, oat starch, potato starch, sweet potato starch, rice starch, pea starch, wheat starch, azuki starch, green bean starch, kudzu starch, Katakuri starch, arrowroot starch, mung bean starch, sago starch, tapioca starch, plant starch (e.g., starch obtained from any of the plants disclosed herein), alginate, chitosan, and derivatives thereof, and mixtures of two or more thereof. In some embodiments, the starch is a modified starch (e.g., pregelatinized starch (e.g., corn, wheat, tapioca), pregelatinized high amylose content starch, pregelatinized hydrolyzed starches (e.g., maltodextrins, corn syrup solids, rice syrup solids, tapioca syrup solids), chemically modified starches such as pregelatinized substituted starches (e.g., octenyl succinate modified starches), alkaline modified starch, bleached starch, oxidized starch, monostarch phosphate, distarch phosphate, phosphated distarch phosphate, acetylated distarch phosphate, acetylated starch, mono starch acetate, acetylated starch, mono starch acetate, acetylated distarch adipate, distarch glycerine, hydroxy propyl starch, hydroxy propyl distarch glycerine, hydroxy propyl distarch phosphate, starch sodium octenyl succinate, acetylated oxidized starch, dextrin, sodium octenylsuccinate starch, and derivatives thereof, and mixtures of two or more thereof), flours (e.g., acorn flour, almond flour, amaranth flour, atta flour, barley flour, bean flour, buckwheat flour, cassava flour, chestnut flour, chufio flour, coconut flour, corn (maize) flour, durum flour, einkorn flour, emmer flour, fava bean flour, garbanzo flour, ground chia seeds, ground flaxseeds, hemp flour, khorasan flour, lentil flour, maida flour, malted barley flour, masa harina, mesquite flour, millet flour, nut flour, oat flour, pea flour, peanut flour, potato flour, quinoa flour, rice flour, rye flour, sorghum flour, soy flour, spelt flour, sweet rice flour, taro flour, teff flour, wheat flour, vital wheat gluten, ground chia seeds, ground flaxseed, and derivatives thereof, and mixtures of two or more thereof), gums (e.g., arrowroot flour, xanthan gum, acacia gum (gum arabic), gellan gum, guar gum, locust bean gum (carob gum), tragacanth gum, carrageenan, tara gum, wheat gum, konjac gum, agar gum, karaya gum, salep, modified cellulose (e.g., methylcellulose, methoxymethylcellulose, hydroxypropyl methylcellulose, carboxymethylcellulose, microcrystalline cellulose), and derivatives and mixtures thereof. In some embodiments, the gum is a modified gum (e.g., deacetylated, deacetylated clarified, partially deacetylated, partially deacetylated clarified, and derivatives thereof, and mixtures of two or more thereof)), edible fibers (e.g., acacia fiber, bamboo fiber, barley bran, carrot fiber, cellulose (e.g., wood pulp cellulose), citrus fiber, corn bran, soluble dietary fiber, insoluble dietary fiber, oat bran, pea fiber, rice bran, head husks, psyllium husk, konjac, soy fiber, soy polysaccharide, wheat bran, inulin, and derivatives thereof, and mixtures of two or more thereof), and mixtures of two or more thereof; and mixtures of two or more thereof.

Non-limiting examples of lipids include fats, oils, monoglycerides, diglycerides, triglycerides, phospholipids, and free fatty acids.

Non-limiting examples of oils include plant oils (e.g., sunflower oil, coconut oil, mustard seed oil, peanut oil, camelina sativa oil, canola oil, corn oil, cottonseed oil, cuphea oil, flax seed oil, olive oil, palm oil, rapeseed oil, safflower oil, sesame oil, soybean oil, almond oil, beech nut oil, brazil nut oil, cashew nut oil, hazelnut oil, macadamia nut oil, mongongo nut oil, pecan oil, pine nut oil, pistachio nut oil, walnut oil, avocado oil, grape oil), microbe-derived oils, algae-derived oils, fungus-derived oils, marine animal oils (e.g., Atlantic fish oil, Pacific fish oil, Mediterranean fish oil, light pressed fish oil, alkaline treated fish oil, heat treated fish oil, light and heavy brown fish oil, bonito oil, pilchard oil, tuna oil, sea bass oil, halibut oil, spearfish oil, barracuda oil, cod oil, menhaden oil, sardine oil, anchovy oil, capelin oil, Atlantic cod oil, Atlantic herring oil, Atlantic mackerel oil, Atlantic menhaden oil, salmonid oil, and shark oil, squid oil, cuttle fish oil, octopus oil, krill oil, seal oil, whale oil), non-essential oils, essential oils, natural oils, non-hydrogenated oils, partially hydrogenated oils, hydrogenated oils (e.g., hydrogenated coconut oil), crude oils, semi-refined (also called alkaline refined) oils, interesterified oils, and refined oils. In some embodiments, longer chain oils (e.g., sunflower oil, corn oil, olive oil, soy oil, peanut oil, walnut oil, almond oil, sesame oil, cottonseed oil, canola oil, safflower oil, flax seed oil, palm oil, palm kernel oil, palm fruit oil, coconut oil, babassu oil, shea butter, mango butter, cocoa butter, wheat germ oil, rice bran oil, engineered sunflower oil that over-expresses oleic acid by 400%) are combined with short-chain triglycerides to produce transesterified fatty acid esters (e.g., to create a specific flavor profile).

Non-limiting examples of monoglycerides and diglycerides include plant-derived monoglycerides and diglycerides, (e.g., monoglycerides and diglycerides derived from sunflower, coconut, peanut, cottonseed, olive, palm, rapeseed, safflower, sesame seed, soybean, almond, beech nut, Brazil nut, cashew, hazelnut, macadameia nut, mongongo nut, pecan, pine nut, pistachio, walnut, and avocado). The monoglycerides and diglycerides may comprise acyl chains of any free fatty acid known in the art, including acyl chains of any free fatty acid disclosed herein.

Non-limiting examples of free fatty acids include butyric acid, caproic acid, caprylic acid, capric acid, lauric acid, myristic acid, palmitic acid, stearic acid, arachidic acid, behenic acid, lignoceric acid, cerotic acid, myristoleic acid, pamitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, omega-fatty acids (e.g., arachidonic acid, omega-3-fatty acids, omega-6-fatty acids, omega-7-fatty acids, omega-9-fatty acids), fatty acids with even number of carbons of 4-16 carbons in length, monosaturated acids (particularly with 18 carbons), fatty acids with low interfacial tension (e.g., less than 20, less than 15, less than 11, less than 9, less than 7, less than 5, less than 3, less than 2, less than 1, or less than 0.5 dynes/cm, from 0.1 to 20, from 1 to 15, from 2 to 9, from 3 to 9, from 4 to 9, from 5 to 9, from 2 to 7, from 0.1 to 5, from 0.3 to 2, or from 0.5 to 1 dynes/cm, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0), butyric (4:0) acid or caproic (6:0) acid that is esterified at sn-3, medium-chain fatty acids (8:0-14:0) as well as 16:0 that are esterified at positions sn-1 and sn-2, fatty acids in which stearic acid (18:0) is placed at position sn-1, fatty acids in which oleic acid (18:1) is placed at positions sn-1 and sn-3, fatty acids that have a range of carbon atoms (e.g, from 8 to 40, from 10 to 38, from 12 to 36, from 14 to 34, from 16 to 32, from 18 to 30, or from 20 to 28 carbon atoms), fatty acids that comprise at least one unsaturated bond (i.e., a carbon carbon double or triple bond; e.g., at least 2, at least 3, at least 4, at least 5, at least 6, at least 7, or at least 8 carbon-carbon double bonds and/or triple bonds), fatty acids with conjugated unsaturated bonds (i.e., at least one pair of carbon-carbon double and/or triple bonds are bonded together, without a methylene (CH2) group between them (e.g., 4CH:CHi CH:CHi)), and derivatives of the above named fatty acids (e.g., esters (e.g., methyl and ethyl esters), salts (e.g., sodium and potassium salts), triglyceride derivatives, diglycerides derivatives, monoglyceride derivatives). The free fatty acids may be saturated or unsaturated. In some embodiments, the free fatty acids are not derived from or produced by a mammal.

Non-limiting examples of phospholipids include lecithin phospholipids (e.g., soy lecithin phospholipids, sunflower lecithin phospholipids, cotton lecithin phospholipids, rapeseed lecithin phospholipids, rice bran lecithin phospholipids, corn lecithin phospholipids, flour lecithin phospholipids), cardiolipin, ceramide phosphocholines, ceramide phosphoethanolamines, glycerophospholipids, phasphatidicacid, phosphatidylcholine, phosphatidylethanolamine, phosphatidylinositol, phosphospingolipids, and phsophatidylserine. In some embodiments, the phospholipids are not derived from or produced by a mammal.

Non-limiting examples of triglycerides include tributyrin, short-chain triglycerides, short-chain triglycerides comprising three oleic acids; short-chain triglycerides comprising hexanoic acid; short-chain triglycerides comprising hexanoic acid and butyric acid; short-chain triglycerides comprising hexanoic acid and decanoic acid; and short-chain triglycerides comprising one butyric, one hexanoic, and one octanoic acid.

Non-limiting examples of essential and semi-essential amino acids include cysteine, methionine, isoleucine, leucine, phenylalanine, tryptophan, and valine.

Non-limiting examples of functional agents include acidulants, buffering agents, shelf life extending agents, pH and/or ionic strength adjusting agents, anti-microbial agents, anti-oxidants, preservatives, emulsifiers, plasticizers, texturing/mouthfeel agents, coloring agents, taste/flavor agents, aroma agents, leavening agents, and flow agents.

Non-limiting examples of shelf life extending agents include carbon monoxide, nitrites, sodium metabisulfite, Bombal, and derivatives and mixtures thereof.

Non-limiting examples of preservatives include p-hydroxybenzoate derivatives, sorbic acid, benzoic acid, nisin, natamycin, and derivatives and mixtures thereof.

Non-limiting examples of emulsifiers include anionic emulsifiers, non-ionic emulsifiers, cationic emulsifiers, amphoteric emulsifiers, bioemulsifiers, steric emulsifiers, Pickering emulsifiers, glycolipids (e.g., trehalose lipids, sophorolipids, rhamnolipids, mannosylerythriol lipids), oligopeptides (e.g., gramicidin S, polymyxin), lipopeptides (e.g., surfactin), phospholipids, fatty acids, neutral lipids, polymeric biosurfactants, amphipathic polysaccharides, lipopolysaccharides, proteins (e.g., pea protein, soy protein, chickpea protein, algae protein, yeast protein, potato protein, lentil protein), mannoprotein, sodium phosphates, calcium stearoyl lactylate, mono- and diacetyl tartaric acid esters of monoglycerides, phospholipids, sorbitan monostearate, magnesium stearate, sodium/potassium/calcium salts of fatty acids, calcium stearoyl di lactate, poly-glycerol esters, sorbitan fatty acid esters, acetic acid esters of monoglycerides, lactic acid esters of monoglycerides, citric acid esters of monoglycerides, polyglycerol esters of fatty acids, polyglycerol polyricinoleate, propane-1,2-diol esters of fatty acids, sugar esters, sucrose esters of fatty acids, monoglycerides, acetylated monoglycerides, lactylated monoglycerides, diglycerides, phosphate monoglycerides, diacetyl tartaric acid esters, sodium/calcium stearoyl-2-lactylate, ammonium phosphatide, polysorbates, polysorbate-80, carboxymethylcellulose (CMC), modulated cellulose, citric acid esters, locust bean gum, guar gum, liposan, emulsan, lecithins (e.g., plant-based lecithins, garbanzo lecithin, fava bean lecithin, soy lecithin, sunflower lecithin, canola lecithin), surfactants (e.g., sorbitan trioleate (Span 85, respectively), sorbitan tristearate (Span 65, respectively), sorbitan sesquioleate (Arlacel 83), glyceryl monostearate, sorbitan monooleate (Span 80), sorbitan monostearate (Span 60), sorbitan monopalmitate (Span 40), sorbitan monolaurate (Span 20), polyoxyethylene sorbitan tristearate (Tween 65, respectively), polyoxyethylene sorbitan trioleate (Tween 85, respectively), polyethylene glycol 400 monostearate, polysorbate 60 (Tween 60), polyoxyethylene monostearate, polysorbate 80 (Tween 80), polysorbate 40 (Tween 40), polysorbate 20 (Tween 20), PEG 20 tristearate, PEG 20 trioleate, PEG 20 monostearate, PEG 20 monooleate, PEG 20 monopalmitate, and PEG 20 monolaurate sorbitan), and derivatives and mixtures thereof.

Non-limiting examples of plasticizers include diethanolamin, triethanolamine, glycerol, sorbitol, PEG-300, PEG-600, urea, octanoic acid, palmitic acid, dibutyl tartrate and phthalate, mono-, di-, or triglycerids esters, fructose, caproic acid, hydrocaproic acid, di-, tri-, or tetra-ethylene glycol, glycerol, 1,3-propane diol, 1,4-butane diol, 1,5-pentane diol, sucrose, and derivatives and mixtures thereof.

Non-limiting examples of texturing/mouthfeel agents include gums (e.g., guar gum, carob gum, wheat gum, xanthan gum), bulking agents, fillers, anti-adherent compounds, dispersing agents, moisture absorbing compounds, chemesthetic agents, film-forming agents, thickening agents, hardening agents, softening agents, stabilizers, anti-caking agents, anti-foaming agents, and derivatives and mixtures thereof.

Non-limiting examples of flavor/aroma agents include ethyl butyrate, 2-furyl methyl ketone, 2,3-pentanedione, γ-undecalactone, 6-undecalactone, propylene glycol, glycerol, ethyl alcohol, dimethylsulfide, 2-methylbutanol, 4-cis-heptenal 2-trans-nonenal, acetone, 2-undecanone, 2-butanone, amyl alcohol, b-decalactone, 2-heptanone, 6-dodecalactone, 2-nonanone, 6-tetradecalactone, hydrogen sulfide, dimethyl sulfone, benzothiazole, 2-pentanone, 2-tridecanone, 6-octalactone, 2-pentadecanone, natural favors, artificial flavors (e.g., chocolate flavoring, coffee flavoring, strawberry flavoring, almond flavoring, hazelnut flavoring, vanilla flavoring, green tea flavoring, Irish cream flavoring, coconut flavoring), sweetening agents (e.g., non-protein sweetening agents, protein-based sweetening agents), and derivatives and mixtures thereof.

Non-limiting examples of non-protein sweetening agents include sugars, modified sugars, natural sweeteners, sweet proteins, artificial sweeteners, sugar alcohols, sugar fibers, sugar extracts including: sucrose, cane juice, corn sugar, high fructose corn syrup, corn sweetener, agave syrup, barley malt syrup, birch syrup, blackstrap molasses, brown rice syrup, caramel, corn sugar, dextrose, douxmatok syrup, coconut palm sugar, fructose, galactose, glucose, glucose fructose syrup, golden syrup, acesulfame potassium, advantame, alitame, aspartame, aspartame-acesulfame salt, cyclamates (e.g., sodium cyclamate), erythritol, fructooligosaccharides, allulose, glucitol (sorbitol), glycerol (glycerin), glycyrrhizin, golden syrup, HFCS-42, HFCS-55, HFCS-90, high maltose corn syrup, honey, date syrup, HSH, hydrogenated starch hydrolysate (HSH), isomaltulose, isomalto-oligosaccharide (IMO), isoglucose, inulin, inverted sugar, isomalt, lactitol, lactose, levulose (fructose), luo han guo (aka monk fruit), maltitol, maltodextrin, maltose, mannitol, maple syrup, molasses, monatin, monellin, neohesperidin dihyrdochalcone, neotame, oligofructose, palm sugar, polydextrose, rapadura, refiners syrup, saccharin, saccharose, sorghum Syrup, stevia, RebM, RebA, RebD, stevioside, sucralose, sucrose, tagatose, osladin, dulcin, glucin, asulfame potassium, L-aspartyl-L-phenylalanine, P-4000, mogrosides, trehalose, xylitol, yacon syrup, and derivatives and mixtures thereof.

Non-limited examples of protein-based sweetening agents include brazzein (UniProt sequence P56552), curculin (UniProt sequence P19667 amino acids 23 to 136, Q6F495 amino acids 23 to 135, respectively), mabinlin (UniProt sequences P80351 amino acids 1 to 32, P80351 amino acids 33 to 104, P30233 amino acids 36 to 68, P30233 amino acids 83 to 154, P80352 amino acids 1 to 32, P80352 amino acids 33 to 104, P80353 amino acids 1 to 28, P80353 amino acids 29 to 100), miraculin (UniProt sequence P13087 amino acids 30 to 220), monelin (UniProt sequences P02881, P02882), pentadin, and thaumatin (UniProt sequences P02883 amino acids 23 to 229, P02884 amino acids 23 to 229, respectively), and homologs and fragments and mixtures thereof.

### Food Product

The composition according to any of the above may be a food product.

The food product may be a supplemented food product (i.e., a conventional food product that is supplemented with the recombinant protein according to any of the above), or may be a substitute food product (i.e., a food product that resembles a conventional food product and that can be used in place of the conventional food product), selected from any of the food product categories defined by the National Health and Nutrition Examination Survey (NHANES).

Non-limiting examples of NHANES food product categories include snack foods and gums (e.g., snack bars, crackers, salty snacks from grain products, chewing gums); breads, grains, and pastas (e.g., oat breads and rolls, cornbread, corn muffins, tortillas, flour and dry mixes, biscuits, multi-grain breads and rolls, whole wheat breads and rolls, pastas, rye breads and rolls, cracked wheat breads and rolls, white breads and rolls); beverages (e.g., beers and ales, beverage concentrates, beverages, energy drinks, sports drinks, fluid replacements, soft drinks, carbonated beverages, juices, wines, beers, cocktails, nutrition drinks, nutrition powders, protein-enriched beverages, coffee, tea); sweets and desserts (e.g., cakes, candies, chips, cookies, cobblers, pastries, ices or popsicles, muffins, pies, sugar replacements or substitutes, syrups, honey, jellies, jams, preserves, salads, crepes, Danish, breakfast pastries, doughnuts); breakfast foods (e.g., cereal grains, cereal, rice, French toast, pancakes, waffles, coffee cake); salad dressings, oils, sauces, condiments (e.g., cooking fats, vegetable oils, salad dressings, tomato sauces, gravies); potatoes (e.g., potato salad, potato soups, chips and sticks, fried potatoes, mashed potatoes, stuffed potatoes, puffs); and soups (e.g., vegetable soups, vegetable broths), meals, main dishes, proteins (e.g., meat substitutes), and seafoods.

The food product according to any of the above may be a supplemented dairy product (i.e., a conventional dairy product that is supplemented with the recombinant protein according to any of the above) or a substitute dairy product (i.e., a food product that resembles a conventional dairy product). The term “dairy product” as used herein refers to milk (e.g., whole milk [at least 3.25% milk fat], partly skimmed milk [from 1% to 2% milk fat], skim milk [less than 0.2% milk fat], cooking milk, condensed milk, flavored milk, goat milk, sheep milk, dried milk, evaporated milk, milk foam), and products derived from milk, including but not limited to yogurt (e.g., whole milk yogurt [at least 6 grams of fat per 170 g], low-fat yogurt [between 2 and 5 grams of fat per 170 g], nonfat yogurt [0.5 grams or less of fat per 170 g], greek yogurt [strained yogurt with whey removed], whipped yogurt, goat milk yogurt, Labneh [labne], sheep milk yogurt, yogurt drinks [e.g., whole milk Kefir, low-fat milk Kefir], Lassi), cheese (e.g., whey cheese such as ricotta; pasta filata cheese such as mozzarella; semi-soft cheese such as Havarti and Muenster; medium-hard cheese such as Swiss and Jarlsberg and halloumi; hard cheese such as Cheddar and Parmesan; washed curd cheese such as Colby and Monterey Jack; soft ripened cheese such as Brie and Camembert; fresh cheese such as cottage cheese, feta cheese, cream cheese, paneer, and curd), processed cheese, processed cheese food, processed cheese product, processed cheese spread, enzyme-modulated cheese; cold-pack cheese), dairy-based sauces (e.g., salad dressing, bechamel sauce, fresh sauces, frozen sauces, refrigerated sauces, shelf stable sauces), dairy spreads (e.g., low-fat spread, low-fat butter), cream (e.g., dry cream, heavy cream, light cream, whipping cream, half-and-half, coffee whitener, coffee creamer, sour cream, crème fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (e.g., milk protein concentrate, whey protein concentrate, demineralized whey protein concentrate, β-lactoglobulin concentrate, α-lactalbumin concentrate, glycomacropeptide concentrate, casein concentrate), milk protein isolate (e.g., milk protein isolate, whey protein isolate, demineralized whey protein isolate, β-lactoglobulin protein isolate, α-lactalbumin protein isolate, glycomacropeptide protein isolate, casein protein isolate), nutritional supplements, texturizing blends, flavoring blends, coloring blends, ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, bars (e.g., nutrition bars, protein bars), and fermented dairy products (e.g., yoghurt, cheese, sour cream, cultured buttermilk, cultured butter, cultured butter oil).

The food product according to any of the above may be a supplemented animal meat or animal meat product (i.e., a conventional animal meat or animal meat product that is supplemented with the recombinant protein according to any of the above produced by the recombinant host cell according to any of the above and/or a method according to any of the above) or a substitute animal meat or animal meat product (i.e., a food product that resembles a conventional animal meat or animal meat product). Non-limiting examples of animal meats and animal meat products include flesh obtained from skeletal muscle or from other organs (e.g., kidney, heart, liver, gallbladder, intestine, stomach, bone marrow, brain, thymus, lung, tongue), or parts thereof, obtained from an animal. The animal meat may be dark or white meat. Non-limiting examples of animals from which animal meat or animal meat product can be obtained include cattle, lamb, mutton, horse, poultry (e.g., chicken, duck, goose, turkey), fowl (e.g., pigeon, dove, grouse, partridge, ostrich, emu, pheasant, quail), fresh or salt water fish (e.g., catfish, tuna, spearfish, shark, halibut, sturgeon, salmon, bass, muskie, pike, bowfin, gar, eel, paddlefish, bream, carp, trout, walleye, snakehead, crappie, sister, mussel, scallop, abalone, squid, octopus, sea urchin, cuttlefish, tunicate), crustacean (e.g., crab, lobster, shrimp, barnacle), game animal (e.g., deer, fox, wild pig, elk, moose, reindeer, caribou, antelope, zebra, squirrel, marmot, rabbit, bear, beaver, muskrat, opossum, raccoon, armadillo, porcupine, bison, buffalo, boar, lynx, bobcat, bat), reptile (e.g., snakes, turtles, lizards, alligators, crocodiles), any insect or other arthropod, rodent (nutria, guinea pig, rat, mice, vole, groundhog, capybara), kangaroo, whale, and seal. The animal meat or animal meat product may be ground, chopped, shredded, or otherwise processed, and uncooked, cooking, or cooked.

The food product according to any of the above may be a supplemented egg product (i.e., a conventional egg or egg product that is supplemented with the recombinant protein according to any of the above) or a substitute egg or egg product (i.e., a food product that resembles a conventional egg or egg product). Non-limiting examples of eggs or egg products include whole egg (e.g., liquid whole egg, spray-dried whole egg, frozen whole egg), egg white (e.g., liquid egg white, spray-dried egg white, frozen egg white), egg yolk, egg dishes, egg soups, mixtures made with egg whites, mixtures made with egg substitutes, mayonnaise, custard, and salad dressings.

The food product according to any of the above may be a pet food or animal feed.

The food product according to any of the above may be essentially free of any protein other than the recombinant protein or the recombinant proteins contained in the composition according to any of the above.

The food product according to any of the above may be essentially free of any recombinant protein other than the recombinant protein or the recombinant proteins contained in the composition according to any of the above.

The food product according to any of the above may be essentially free of any recombinant milk protein other than the recombinant protein or the recombinant proteins contained in the composition according to any of the above.

The food product according to any of the above may be essentially free of a component found in a mammal-produced milk (e.g., cow milk, goat milk, sheep milk, human milk, buffalo milk, yak milk, camel milk, llama milk, alpaca milk, horse milk, donkey milk), or may comprise a lower concentration of at least one component found in a mammal-produced milk. Non-limiting examples of components found in mammal-derived milk include lactose, saturated fat, cholesterol, native milk proteins, and native milk lipids. The food product may be essentially free of any milk protein other than the milk protein comprised in the recombinant protein or the recombinant proteins contained in the composition according to any of the above.

The food product according to any of the above may be essentially free of a component obtained from an animal (i.e., a component that is native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk, milk lipid extracted from mammal-derived milk, animal lipids, animal proteins]), or comprise 2% or less by mass of such component.

### Cosmetic or Personal Care Product

The composition according to any of the above may be a cosmetic or personal care composition.

The term “cosmetic or personal care composition” as used herein refers to a composition that upon application to a body surface (i.e., an exposed area of a human body, such as skin, hair, nail, tooth, and tissues of the oral cavity [e.g., gums]) confers a perceived or actual beautifying or hygienizing effect. Non-limiting examples of cosmetic or personal care compositions include anti-wrinkling treatments (i.e., compositions used for tensioning [e.g., smoothing out of skin, reducing wrinkles in skin, removing tine lines in skin]), anti-aging treatments (i.e., compositions used for removing signs of aging [e.g., wrinkles, fine lines, manifestations of photodamage (e.g., sun spots)]), sun protection (i.e., compositions used to protect against UV exposure), anti-burn treatments (i.e., compositions used for soothing burns [e.g., sunburns]), anti-acne treatments (i.e., compositions that are effective in the treatment of acne and/or the symptoms associated therewith), skin cleansers (i.e., compositions used for cleaning skin and/or skin pores [e.g., nose strips for pore cleaning]), anti-dandruff treatments (i.e., compositions used for reducing or eliminating dandruff), anti-body odor treatments (i.e., compositions used for reducing or eliminating body odor), self-tanning treatments (i.e., compositions used for darkening skin color), skin whitening treatments (i.e., compositions used for bleaching/depigmenting skin color), hair coloring treatments (i.e., compositions used for coloring hair), lotions (e.g., skin lotions, body care lotions, wash lotions, moisture retention lotions, pre-shave lotions, after-shave lotions), pastes (e.g., washing pastes), ointments, balms, salves, masks, creams (e.g., water in oil creams, oil in water creams, day creams, night creams, eye creams, skin creams, face creams, anti-wrinkle creams, sun protection creams, moisture retention creams, after-shave creams, skin bleaching creams, self-tanning creams, vitamin creams, moisturizing creams, massage creams), milks (e.g., body milks, cleansing milks), gels (e.g., anhydrous gels, shower gels), Eau de Toilette, soaps (e.g., transparent soaps, luxurious soaps, deodorant soaps, cream soaps, baby soaps, skin protection soaps, abrasive soaps, syndets, pasty soaps, soft soaps, peeling soaps), skin peeling treatments, liquid washes, shower and bath preparations (e.g., wash lotions, shower baths, shower gels, foam baths, oil baths, scrub preparations), foams (e.g., shaving foams, foam baths), deodorants, hair care products (e.g., shampoos, conditioners, hair mousses, hair colorants, hair sprays, rinse-off lotions, hair gels, hair emulsions, hair laquers, hair tonics), lip glosses, sprays (e.g., hair sprays, pump sprays, sprays containing blowing agents), treatments for skin defects (e.g., dermatitis, weals, chaps, blemishes, cracks, scars, freckles, moles, rashes, blisters, pustules), toners, cleaning tissues, sanitary towels, tampons, nappies, repellents, make-up products (e.g., studio pigments, mascara, eye shadows, eyeliners, eye liner pens, rouges, face powders, eyebrow pencils, lipsticks, foundations, tinted creams, concealer sticks, blemish sticks, blushes), sticks (e.g., lipsticks, concealer sticks, blemish sticks), hair removing agents, hand cleaning products, intimate hygiene products, foot care products, baby care products, and oral hygiene products (e.g., chewing gums, mouthwashes, toothpastes, gum-cleaning agents, denture adhesives, denture fixatives).

The cosmetic or personal care composition according to any of the above may be essentially free of any protein other than the recombinant protein or the recombinant proteins contained in the composition according to any of the above.

The cosmetic or personal care composition according to any of the above may be essentially free of any recombinant protein other than the recombinant protein or the recombinant proteins contained in the composition according to any of the above.

The cosmetic or personal care composition according to any of the above may be essentially free of any recombinant milk protein other than the recombinant protein or the recombinant proteins contained in the composition according to any of the above.

The cosmetic or personal care composition according to any of the above may be essentially free of a component found in a mammal-produced milk (e.g., cow milk, goat milk, sheep milk, human milk, buffalo milk, yak milk, camel milk, llama milk, alpaca milk, horse milk, donkey milk), or may comprise a lower concentration of at least one component found in a mammal-produced milk. Non-limiting examples of components found in mammal-derived milk include lactose, saturated fat, cholesterol, native milk proteins, and native milk lipids. The cosmetic or personal care composition may be essentially free of any milk protein other than the milk protein comprised in the recombinant protein or the recombinant proteins contained in the composition according to any of the above.

The cosmetic or personal care composition according to any of the above may be essentially free of a component obtained from an animal (i.e., a component that is native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk, milk lipid extracted from mammal-derived milk, animal lipids, animal proteins]), or comprise 2% or less by mass of such component.

The cosmetic or personal care composition according to any of the above may be essentially free of a component derived from petroleum.

### Method for Producing Composition

In various aspects, provided herein is a method for producing a composition according to any of the above (e.g., food product according to any of the above, cosmetic or personal care composition according to any of the above), wherein the method comprises: obtaining the recombinant protein according to any of the above.

When the composition is a food product (e.g., the food product according to any of the above), a variety of recipes are known in the art may be used to prepare the food product. The recombinant protein according to any of the above may be used in such recipes in purified form or comprised in a fermentation broth or preparation obtained in a method according to any of the above.

### Attributes

The recombinant protein, polymer, or composition according to any of the above may have various attributes.

The attribute may be crystallinity (e.g., crystallinity that provides for a specific translucence, opaqueness, or transparency). For example, a higher crystallinity generally allows less light to pass through a polymer, affecting translucence or opaqueness of the polymer, as well as mechanical strength, stiffness, chemical resistance, and stability. Methods for measuring crystallinity are known in the art, and include differential scanning calorimetry (DSC) (see, for example, B. Wunderlich, Thermal Analysis, Academic Press, 1990, pp. 417-431.; TN 48, “Polymer Heats of Fusion”, TA Instruments, New Castle, DE; Ramos et al. 2012 Critical Reviews in Food Science and Nutrition 52(6):533-552).

The attribute may be viscosity and/or density. Methods for measuring viscosity and density are known in the art, and include viscometric methods, rotational viscometric methods, capillary viscometric methods, vibratory viscometric methods, ultrasonic pulse echo methods, pycnometric methods, densymetric methods, and areometric methods (see, for example, Kazys & Rekuviene 2011 Ultragarsas (Ultrasound) 66(4):20-25).

The attribute may be solubility (i.e., amount of polymer that is dispersed and colloidally stable under given conditions and in various solvents). Methods for measuring solubility are known in the art, and include optical measurement of destabilization over time, measurement of sedimentation layer thickness, and measurements of quantitating amount of dispersed protein after gravimetric separation.

The attribute may be foaming/leavening capacity. Methods for measuring foaming capacity are known in the art, and include measurement of percentage of air incorporated in a foam formed after whipping at a specified speed and for a specified amount of time under defined conditions (e.g., temperature, pH, ionic strength, protein concentration, carbohydrate concentration), measurement of how long it takes for a given mass of foam to destabilize in the form of liquid draining or seeping, and measurement of yield stress under shear or the amount of stress required to initiate flow in a sample (see, for example, PCT publication WO2020219595, published Oct. 29, 2020).

The attribute may be gelling/thickening/coagulating capacity. Methods for measuring gelling/thickening capacity are known in the art, and include measurement of the time required to form a gel under defined conditions (e.g., temperature, pH, ionic strength, protein concentration, carbohydrate concentration), measurement of storage and elastic moduli and phase angle obtained in frequency sweeps on a rheometer, and measurement of resistance of a gel to a physical force and/or chemical condition (e.g., agitation, temperature, pH, ionic strength, protein concentration, sugar concentration) (see, for example, PCT publication WO2020219595, published Oct. 29, 2020).

The attribute may be gelling profile. Methods for measuring gelling profile are known in the art (see, for example, Stable Micro Systems Ltd. 2020. How to Measure the Physical Properties of Gels & Films. AZoM, https://www.azom.com/article.aspx?ArticleID=19349).

The attribute may be water binding capacity. The water holding capacity may be between about 80% and about 120% of that of a casein. Methods for measuring water holding capacity are known in the art, and include capillary viscometry, measurement of amount of water exuded after centrifugation, or development of moisture sorption isotherms based on mass of water bound per mass of protein as a function of vapor pressure).

The attribute may be emulsifying capacity. Methods for measuring emulsifying capacity are known in the art, and include measurement of surface excess concentration, measurement amount of oil emulsified by a given mass of protein using gravimetric methods to determine the, measurement of stability over time of phase separation in a mixture of lipid and water, measurement of rate of creaming or sedimentation, measurement of change in opacity over time, measurement of change of dispersed phase particle size over time, and measurement of interfacial properties.

The attribute may be shelf life (i.e., maintenance/stability of color and appearance over time [e.g., 2 years]). Methods for measuring color and appearance maintenance capacity are known in the art (see, for example, Ramos et al. 2012 Critical Reviews in Food Science and Nutrition 52(6):533-552).

The attribute may be heat stability. The heat stability may be a heat stability of at least 80° C., and/or a heat stability that is similar to that of the milk protein 1, the milk protein 2, the milk protein 3, or the milk protein 4 unit with the lowest melting temperature (Tm). Methods for measuring stability are known in the art, and include measurement of changes during/after heating, such as changes in particle size, changes in particle properties, changes in optical properties (e.g., opacity, turbidity, clarity), changes in solubility, changes in resistance to gravimetric separation, changes in viscosity, and/or changes in resistance to fouling/film formation. Methods for measuring thermal stability of a foam can be studied are known in the art (see, for example, Ye et al. 2021 Adv. Sustainable Syst. 2100063)

The attribute may be elasticity (i.e., ability to return to original shape after stretching or other alteration as a result of application of force). Methods for measuring elasticity are known in the art (see, for example, Cao and Mezzenga, 2020 Nat Food 1:106-118).

The attribute may be tensile strength (i.e., maximum load that a material can support without fracture when being stretched, divided by original cross-sectional area of the material). Methods or measuring tensile strength are known in the art (see, for example, Ramos et al. 2012 Critical Reviews in Food Science and Nutrition 52(6):533-552, Mundada et al. 2011 Iran J Pharm Res. 10(1):35-42).

The attribute may be elongation (i.e., maximum change in length before breaking, expressed as percent change of the original length of the material). Methods or measuring elongation are known in the art (see, for example, Olivas and Barbosa-Canovas, 2005 Crit Rev Food Sci Nutr. 45(7-8):657-670).

The attribute may be Young modulus (i.e., ratio of stress to strain, in the initial linear part of the stress/strain curve). Methods or measuring Young modulus are known in the art (see, for example, McHugh and Krochta, 1994 J. Agric. Food Chem. 42:41-45).

The attribute may be tear resistance (i.e., ability to withstand effects of tearing). Methods or measuring tear resistance are known in the art (see, for example, Krochta 2002 Protein-based Films and Coatings, pp. 1-41).

The attribute may be biodegradability (i.e., able to be metabolize into harmless product). Methods for measuring biodegradability are known in the art, and include OECD 306 Biodegradability in Sea Water, OECD 311 (ASTM E2170) Anaerobic Biodegradability/Biochemical Methane Potential, ASTM D5338 Aerobic biodegradability/composting assay, ASTM D5511 Anaerobic biodegradability/“landfill simulation”, ASTM D5988 (ISO17556) Biodegradation in soil.

When the composition is a food product, the attribute may be eating quality (e.g., flavor, mouthfeel, fattiness, creaminess, richness, greasiness, thickness, hardness/firmness, crispiness, crumbliness, crunchiness, chewiness, chewdown, tenderness, compactness, cohesiveness, adhesiveness, graininess, smoothness, juiciness, wetness, mouthcoating, slipperiness on tongue, roughness, abrasiveness, uniformity of bite and/or chew, springiness, texture, airiness, effort required to draw sample from spoon over tongue), nutrient content (e.g., types and/or amounts of proteins, types and/or amounts of amino acids [e.g., branched amino acids], PDCAAS, BV, types and/or amounts of lipids, types and/or amounts of minerals, types and/or amounts of vitamins), spreadability, taste imparting capacity, gastro-intestinal absorption, gastro-intestinal digestibility, oxidative stability, ability to form micelles (i.e., generally or roughly spherical supramolecular structure that exist as a dispersion within a composition and that may encapsulate one or more biomolecules [e.g., water, minerals, vitamins)), interaction with other proteins, hunger and/or satiety regulation capacity, and use versatility (i.e., potential for varied use and production of a diversity of food products; e.g., ability to produce food products that resemble substitute dairy products [e.g., any of the substitute dairy products disclosed herein]).

When the composition is a film or coating, the attribute may be cohesiveness of surface morphology and/or adhesiveness to a surface (e.g., a surface of a food product). Methods for measuring cohesiveness and adhesiveness are known in the art, and include scanning electron microscopy (see, for example, Choi et al. 2002 J. Food. Sci. 66:2668-2672) and confocal Raman microspectrometry, surface enhanced Raman scattering, and Fourier transform Raman spectrometry (see, for example, Hsu et al. 2005 Mat. Sci. Eng. 25:282-290).

When the composition is a film or coating, the attribute may be brittleness and/or stiffness (e.g., dart strength, puncture resistance, peel strength). Methods for measuring brittleness and/or stiffness are known in the art (see, for example, Krochta, 2002 Protein-based Films and Coatings, pp. 1-41).

When the composition is a film or coating, the attribute may be gas permeability (i.e., rate of gas transmission through a unit area of flat material of unit thickness induced by a unit pressure difference between the two sides of the material, under specified temperature and humidity conditions). Methods for measuring gas permeability are known in the art, and include ASTM, 1988 for measurement of oxygen permeability, and modifications of the method employed to measure water vapor permeability for measurement of CO2 permeability (see, for example, Ayranci et al., 1999 J. Sci. Food Agric. 79:1033-1037).

When the composition is a film or coating, the attribute may be water vapor permeability (i.e., rate of water vapor transmission through a unit area of flat material of unit thickness induced by a unit pressure difference between the two sides of the material, under specified temperature and humidity conditions). Methods for measuring water vapor permeability are known in the art, and include ASTM E96-95 (ASTM, 1996).

When the composition is a film or coating, the attribute may be aroma compound permeability (i.e., rate of aroma compound transmission (e.g., 8-limonene transmission) through a unit area of flat material of unit thickness induced by a unit pressure difference between the two sides of the material, under specified temperature and humidity conditions). Methods for measuring aroma compound permeability are known in the art (see, for example, Chalier et al. 2006 Developments Food Science 43:437-440, Perez-Gago and Krochta 2002 Protein-Based Films and Coatings, pp. 159-180).

When the composition is a film or coating, the attribute may be optical property such as a color or a transparency (i.e., % light transmission, haze, and/or transparency). Methods for measuring optical properties are known in the art (see, for example, Abbot 1999 Postharvest Biol. Technol. 15:207-225, Sakai 1998 Konica Minolta Sensing, pp. 3-91). The transparency of films is often determined according to ASTM D1746 (ASTM, 1997, Annual Book of ASTM Standards, pp. 389-391).

When the composition is a gel, the attribute may be swelling rate (e.g., upon immersion in liquids). Methods for measuring swelling rate are known in the art (see, for example, Gunasekaran et al. 2007 Journal of Food Engineering 83:31-40).

When the composition is a gel, the attribute may be gel rheology. The gel rheology may be between about 80% and about 120% of that of gelatin. Methods for measuring gel rheology are known in the art, and include measuring viscoelasticity, storage moduli, complex moduli, phase angle, loss moduli, time dependent behavior, creep, relaxation, and recovery (see, for example, Tien et al. 2000 J. Agric. Food Chem. 48:5566-5575, Mundada et al. 2011 Iran J Pharm Res. 10(1):35-42).

When the composition is a gel, the attribute may be gel strength. Methods for measuring gel strength are known in the art, and include penetrometry and oscillatory rheology.

When the composition is a foam, the attribute may be foam density/porosity and/or foam heat stability. Methods for measuring foam density/porosity and/or heat stability are known in the art (see, for example, Ye et al. 2021 Adv. Sustainable Syst. 2100063, Gunasekaran et al. 2007 Journal of Food Engineering 83:31-40).

When the composition is a foam, the attribute may be foam stability. Methods for measuring foam stability are known in the art, and include measurement of how long it takes for a given mass of foam to destabilize in the form of liquid draining or seeping, and measurement of yield stress under shear or the amount of stress required to initiate flow in a sample (see, for example, PCT publication WO2020219595, published Oct. 29, 2020).

When the composition is an emulsion, the attribute may be emulsion stability (i.e., half-life of an emulsion produced under given conditions, such as, for example, a given protein concentration, lipid concentration, pH, ionic strength, or preparation method). Methods for measuring emulsion stability are known in the art, and include visual inspection over time, measurement of particle size over time, gravimetric methods (creaming or sedimentation over time), and optical methods (e.g., measurement of light transmission or backscattering over time).

The attribute may also be selected from a color, shape (e.g., length, width, uniformity), shape retention, structure (e.g., molecular structure [e.g., protein folding/conformation], air cell average size, air cell size distribution, air cell wall thickness), plasticity, allergenicity, texture, thickness, smoothness, chemical reactivity, melting temperature, toughness, creep or cold flow, porosity, impact resistance, electrical conductivity, thermal conductivity, flexibility, surface active properties (e.g., surface exposed functional groups, surface hydrophobicity, surface charge, surface tension), strength-at-break, glass transition temperature, release of an associated/bound compound, non-toxicity, and/or shaping temperature.

### EXAMPLES

The following examples are included to illustrate specific embodiments of this disclosure. The techniques disclosed in the examples represent techniques discovered by the inventors to function well in the methods and processes of this disclosure; however, those of skill in the art should, in light of the present disclosure, appreciate that many changes can be made in the specific embodiments that are disclosed and still obtain a like or similar result without departing from the spirit and scope of the disclosure. Therefore, all matter set forth or shown in the examples is to be interpreted as illustrative and not in a limiting sense.

### Example 1: Production of Recombinant Protein

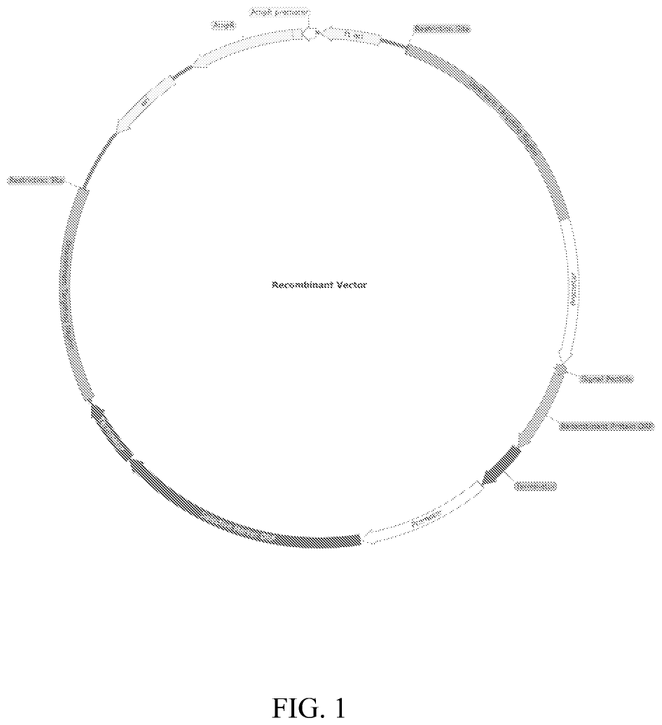

For recombinant protein expression in Trichoderma (e.g., Trichoderma reesei), Aspergillus (e.g., Aspergillus niger), and Pichia (e.g., Pichia pastoris [Komagataella phaffii], strain BG12 [(Biogrammatics, Carlsbad, CA]) host cells, the recombinant vector shown in FIG. 1 is constructed using genetic engineering methods known in the art. The recombinant vector comprises a recombinant expression construct comprising a protein coding sequence encoding a recombinant protein according to any of the above (“recombinant protein ORF”), codon-optimized for expression in the host cells, and operably linked to a N-terminal secretion signal sequence (e.g., pre or pre-pro signal peptide of proteins CBH1 or XYN1 for Trichoderma host cells; pre or pre-pro signal peptide of protein GLAA for Aspergillus host cells; pre or pre-pro signal peptide of Saccharomyces cerevisiae alpha mating factor for Pichia host cells); and under control of a suitable promoter (e.g., promoter sequence of cbh1, xyn1, or tef1 gene for Trichoderma host cells; promoter sequence of glaA or gpdA gene for Aspergillus host cells; promoter sequence of gap or aox1 gene for Pichia host cells) and a terminator (e.g., terminator sequence of pdc1 gene for Trichoderma host cells; terminator sequence of glaA or gpdA gene for Aspergillus host cells; terminator sequence of aox1 gene for Pichia host cells). The recombinant vector further comprises a polynucleotide that can direct integration of the expression construct into the genome of the host cell (e.g., into the cbh1 or egl1 locus for Trichoderma host cells; into the glaA locus for Aspergillus host cells; into the aox1 locus for Pichia host cells), selection markers for selection of bacterial and/or fungal transformants, and a bacterial origin of replication. The bacterial selection markers and origin of replication are removed from the recombinant vector via restriction enzyme digestion prior to transformation of the recombinant vector into the host cells.

The recombinant vector is transformed into Trichoderma, Aspergillus, or Pichia host cells, and transformants are selected by growth on minimal media or antibiotics for positive selection. The transformants are cultured in 24-well plates, and culture supernatants are harvested for identification of recombinant host cells comprising an integrated copy of the expression construct and secreting a recombinant protein by SDS-PAGE gel analyses.

The recombinant host cells are fermented in a stirred fermentation vessel under conditions that permit cell growth and production of the recombinant protein. The fermentation is harvested after at least 100 hours, at a biomass concentration of between about 20 g and about 50 g dry cell weight (DCW) per L. The biomass is removed from the broth by centrifugation at 5,000×g.

The culture supernatant is concentrated over a membrane with suitable molecular weight cutoff. The concentrate retentate is diafiltered over 5 kda MWCO membranes into 50 mM Imidazole, pH 6.8. The concentrated retentate is passed over a Q sepharose FF column. The mobile phase is 50 mM Imidazole, pH 6.8, and the recombinant protein is eluted on a 2M NaCl gradient. The gradient is run from 0-30% over 30 column volumes. Peak fractions are collected and analyzed on RP-HPLC. Peaks containing recombinant protein with a purity of greater than 85% are pooled for final diafiltration into water.

### Example 2: Production of Recombinant Casein

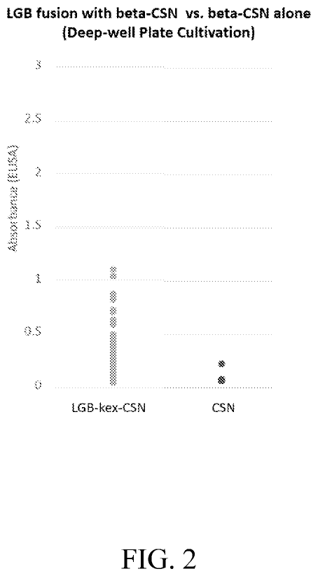

The recombinant vector shown in FIG. 1 was constructed using genetic engineering methods known in the art. The recombinant vector comprised an expression construct comprising a protein coding sequence encoding either Bos taurus β-casein (amino acids 16 to 224 of UniProt sequence P02666), or encoding a recombinant protein having a structure (A) according to the above wherein the milk protein 1 was Bos taurus β-lactoglobulin (amino acids 17-178 of UniProt sequence P02754), the linker peptide 1 had an amino acid sequence of NVISKR (comprising a kex2 endoprotease recognition or cleavage site), and the milk protein 2 was Bos taurus β-casein (amino acids 16 to 224 of UniProt sequence P02666), codon-optimized for expression in Aspergillus niger, operably linked to an N-terminal secretion signal (e.g., pre or pre-pro signal peptide of protein GLAA); and under control of a suitable promoter (e.g., promoter sequence of glaA or gpdA gene) and a suitable terminator (e.g., terminator sequence of glaA or gpdA gene). The recombinant vector further comprised a polynucleotide that can direct integration of the expression construct into the genome of an Aspergillus niger host cell (e.g., at the glaA locus), selection markers for selection of bacterial and/or fungal transformants, and a bacterial origin of replication. The bacterial selection markers and origin of replication were removed from the recombinant vector via restriction enzyme digestion prior to transformation of the recombinant vector into the host cell. The recombinant vector was transformed into Aspergillus niger host cells, and transformants were selected by growth on minimal media or antibiotics for positive selection. The transformants were cultured in deep-well plates, and culture supernatants were analyzed using ELISA with an anti-β-casein antibody. As shown in FIG. 2, when produced as a recombinant fusion protein with β-lactoglobulin, production and/or stability of β-casein was increased.

### Example 3: Determination of Melting Temperature (Tm) of Recombinant Protein

The melting temperature (Tm) of the recombinant protein of Example 1 is determined by differential scanning calorimetry (DSC). To this end, a cell comprising the recombinant protein is heated, and its thermogram is compared to that of a reference sample to deduce insight into the thermal stability of the recombinant protein (as bond interactions (e.g. noncovalent, ionic, Van der Waals, hydrostatic) in the recombinant protein will absorb a certain amount of energy before finally breaking), and to define the Tm of the recombinant protein (i.e., thermal midpoint transition).

### Example 4: Production of Polymer Comprising Recombinant Protein Monomers

The recombinant protein of any of Example 1 is combined with a weak acid (e.g., 5% acetic acid) or base (e.g., 5% sodium bicarbonate, sodium hydroxide, potassium hydroxide, calcium hydroxide) alone or in combination with an other ingredient (e.g., any of the other ingredients disclosed herein) to a final concentration of between about 2% and about 18% by mass of the recombinant protein, a final pH of between about 4 and about 11, and a final conductivity of between about 10 ms/cm and about 300 mS/cm. The mixture is heated for 20 minutes at a temperature and a pH at which the recombinant protein is mildly denatured (e.g., at which less than 20% of the recombinant protein is denatured), such that the free thiol group(s) of the recombinant protein are exposed but existing intra-molecular disulfide bonds are not broken (e.g., at between 10° C. and 20° C. below the Tm of the recombinant protein [as determined in Example 3]). The mixture is then cooled to 21° C. or ambient temperature. The polymer is captured by centrifugation (e.g., at 4,000 g for 20 min), filtration, solvent extractions, chromatography, or other method. The polymer is dried to a moisture content that still permits shaping. After shaping, the polymer is further dried to set a final form.

### Example 5: Extrusion of Recombinant Protein

The recombinant protein of any of Example 1 is heated as described in Example 4 to obtain mildly denatured recombinant protein. The recombinant protein is then compounded with 0%, 10%, or 30% by weight of Al₂O₃ in a co-rotating twin-screw extruder (e.g., Berstorff ZE 25(CL) 40D) in the presence of 10% by weight of glycerol and 30% by weight of water. Further other ingredients include TCP and/or coupling agent NZ12 (e.g., at 1% by weight). The extruded compound (in the form of pellets) is molded into ASTM tensile test bars (24 mm\hat{ }2 cross section), after being conditioned (60° C. for 24 hours) until the respective moisture content reaches 10-12%. The specimen is molded using, for example, a DEMAG D25 NC 4, under optimized processing conditions. The injection-molded sample is conditioned at 23° C. and 60% relative humidity (RH) for at least 1 week.

### Example 6: Production of Gel Comprising Recombinant Protein

A cylindrical (10-mm inner diameter, 40-mm length) stainless steel tube is filled with a solution comprising between about 2% and about 18% by mass of the recombinant protein at a final pH of between about 4 and about 11. The tube is closed with rubber stoppers, sealed with vinyl electrical tape, and placed vertically in a water bath. The solution is heated as described in Example 4 to obtain mildly denatured recombinant protein. The gel is cooled to room temperature, and then left at 4° C. overnight, before being removed from the tube and cut into 10-mm diameter, 2-mm thick tablets, and dried in an enclosed desiccator until the tablets reach a constant mass (i.e., within ±0.001 g).

### Example 7: Production of Film/Sheet Comprising Recombinant Protein

A solution comprising between about 20% and about 40% by weight of the recombinant protein of Example 1 is heated as described in Example 4 to obtain mildly denatured recombinant protein. The mildly denatured recombinant protein is optionally combined with a synthetic co-polymer (e.g., polyvinyl acetate [PVAC], polyvinyl alcohol [PVA], polyvinyl pyrollidone [PVP]). The solution is then rapidly cooled in an ice water bath. The solution is kept overnight at 4° C. to remove air bubbles. Finally, the solution is cast on the center of 27×27 cm² glass plates then dried for 72 hours at ambient conditions (25° C.).

1. A protein, wherein the protein is a recombinant protein having a structure:
(A) (milk protein 1)-(linker peptide 1) 6-(milk protein 2),
or
(B) (milk protein 1)-(linker peptide 1)-(milk protein 2)-(linker peptide 2)-(milk protein 3),
or
(C) (milk protein 1)-(linker peptide 1)-(milk protein 2)-(linker peptide 2)-(milk protein 3)-(linker peptide 3)-(milk protein 4);
wherein:
(i) the milk protein 1, the milk protein 2, the milk protein 3, the milk protein 4, the linker peptide 1, the linker peptide 2, and the linker peptide 3 are linked via peptide bonds; and
(ii) at least one of the milk protein 1, the milk protein 2, the milk protein 3, and the milk protein 4 is a milk protein comprising a free thiol group.

2. The recombinant protein of claim 1, wherein
(i) the recombinant protein has a structure (A),
(ii) the milk protein 1 is a milk protein comprising a free thiol group, and
(iii) the milk protein 2 is a milk protein not comprising a free thiol group.

3. The recombinant protein of claim 1, wherein
(i) the recombinant protein has a structure (B),
(ii) the milk protein 1 is a milk protein comprising a free thiol group, and
(iii) each of the milk protein 2 and the milk protein 3 is a milk protein not comprising a free thiol group.

4. The recombinant protein of claim 1, wherein
(i) the recombinant protein has a structure (B),
(ii) the milk protein 2 is a milk protein comprising a free thiol group, and
(iii) each of the milk protein 1 and the milk protein 3 is a milk protein not comprising a free thiol group.

5. The recombinant protein of claim 1, wherein
(i) the recombinant protein has a structure (C),
(ii) the milk protein 1 is a milk protein comprising a free thiol group, and
(iii) each of the milk protein 2, the milk protein 3, and the milk protein 4 is a milk protein not comprising a free thiol group.

6. The recombinant protein of claim 1, wherein
(i) the recombinant protein has a structure (C),
(ii) the milk protein 2 is a milk protein comprising a free thiol group, and
(iii) each of the milk protein 1, the milk protein 3, and the milk protein 4 is a milk protein not comprising a free thiol group.

7. The recombinant protein of claim 1, wherein
(i) the recombinant protein has a structure (A), and
(ii) each of the milk protein 1 and the milk protein 2 is a milk protein comprising a free thiol group.

8. The recombinant protein of claim 1, wherein
(i) the recombinant protein has a structure (B),
(ii) each of the milk protein 1 and the milk protein 2 is a milk protein comprising a free thiol group, and
(iii) the milk protein 3 is a milk protein not comprising a free thiol group.

9. The recombinant protein of claim 1, wherein
(i) the recombinant protein has a structure (B),
(ii) each of the milk protein 1 and the milk protein 3 is a milk protein comprising a free thiol group, and
(iii) the milk protein 2 is a milk protein not comprising a free thiol group.

10. The recombinant protein of claim 1, wherein
(i) the recombinant protein has a structure (C),
(ii) each of the milk protein 1 and the milk protein 2 is a milk protein comprising a free thiol group, and
(iii) each of the milk protein 3 and the milk protein 4 is a milk protein not comprising a free thiol group.

11. The recombinant protein of claim 1, wherein
(i) the recombinant protein has a structure (C),
(ii) each of the milk protein 1 and the milk protein 3 is a milk protein comprising a free thiol group, and
(iii) each of the milk protein 2 and the milk protein 4 is a milk protein not comprising a free thiol group.

12. The recombinant protein of claim 1, wherein
(i) the recombinant protein has a structure (C),
(ii) each of the milk protein 1 and the milk protein 4 is a milk protein comprising a free thiol group, and
(iii) each of the milk protein 2 and the milk protein 3 is a milk protein not comprising a free thiol group.

13. The recombinant protein of claim 1, wherein
(i) the recombinant protein has a structure (C),
(ii) each of the milk protein 2 and the milk protein 3 is a milk protein comprising a free thiol group, and
(iii) each of the milk protein 1 and the milk protein 4 is a milk protein not comprising a free thiol group.

14. The recombinant protein of claim 1, wherein
(i) the recombinant protein has a structure (B), and
(ii) each of the milk protein 1, the milk protein 2, and the milk protein 3 is a milk protein comprising a free thiol group.

15. The recombinant protein of claim 1, wherein
(i) the recombinant protein has a structure (C),
(ii) each of the milk protein 1, the milk protein 2, and the milk protein 3 is a milk protein comprising a free thiol group, and
(iii) the milk protein 4 is a milk protein not comprising a free thiol group.

16. The recombinant protein of claim 1, wherein
(i) the recombinant protein has a structure (C),
(ii) each of the milk protein 1, the milk protein 2, the milk protein 4 is a milk protein comprising a free thiol group, and
(iii) the milk protein 3 is a milk protein not comprising a free thiol group.

17. The recombinant protein of claim 1, wherein
(i) the recombinant protein has a structure (C), and
(ii) each of the milk protein 1, the milk protein 2, the milk protein 3, and the milk protein 4 is a milk protein comprising a free thiol group.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method for producing a casein wherein the method comprises:
a) fermenting a recombinant host cell capable of producing a recombinant protein having a structure
(A) (milk protein 1)-(linker peptide 1)-(milk protein 2),
or
(B) (milk protein 1)-(linker peptide 1)-(milk protein 2)-(linker peptide 2)-(milk protein 3),
or
(C) (milk protein 1)-(linker peptide 1)-(milk protein 2)-(linker peptide 2)-(milk protein 3)-(linker peptide 3)-(milk protein 4);
wherein:
(i) the milk protein 1, the milk protein 2, the milk protein 3, the milk protein 4, the linker peptide 1, the linker peptide 2, and the linker peptide 3 are linked via peptide bonds,
(ii) at least one of the milk protein 1, the milk protein 2, the milk protein 3, and the milk protein 4 is a β-lactoglobulin,
(iii) at least one of the milk protein 1, the milk protein 2, the milk protein 3, and the milk protein 4 is the casein, and
(iv) wherein the casein is adjacent to one or more linker peptides comprising a recognition or cleavage sequence for a protease.

32. The method of claim 31, wherein
(i) the recombinant protein has a structure (A),
(ii) the milk protein 1 is a β-lactoglobulin,
(iii) the milk protein 2 is the casein, and
(iv) the linker peptide 1 comprises a recognition or cleavage sequence for a protease.

33. The method of claim 31, wherein
(i) the recombinant protein has a structure (B),
(ii) at least one of the milk protein 1 and the milk protein 2 is a β-lactoglobulin,
(iii) the milk protein 2 is the casein, and
(iv) the linker peptide 1 and the linker peptide 2 comprise a recognition or cleavage sequence for a protease.

34. The method of claim 31, wherein
(i) the recombinant protein has a structure (C),
(ii) at least one of milk protein 1 and milk protein 4 is a β-lactoglobulin,
(iii) the milk protein 2 and the milk protein 3 is the casein, and
(iv) the linker peptide 1, the linker peptide 2, and the linker peptide 3 comprise a recognition or cleavage sequence for a protease.

35. The method of claim 31, wherein
(i) the recombinant protein has a structure (C),
(ii) at least one of milk protein 1 and milk protein 3 is a β-lactoglobulin,
(iii) the milk protein 2 and the milk protein 4 is the casein, and
(iv) the linker peptide 1, the linker peptide 2, and the linker peptide 3 comprise a recognition or cleavage sequence for a protease.

36. The method of claim 31, wherein
(i) the recombinant protein has a structure (C),
(ii) the milk protein 1 is a β-lactoglobulin,
(iii) the milk protein 2, the milk protein 3, and the milk protein 4 is the casein, and
(iv) the linker peptide 1, the linker peptide 2, and the linker peptide 3 comprise a recognition or cleavage sequence for a protease.

37. The method of claim 31, wherein the casein is k-casein.

38. The method of claim 31, wherein the casein is b-casein.

39. The method of claim 31, wherein the casein is a-S1-casein.

40. The method of claim 31, wherein the casein is a-S2-casein.

41. The method of claim 31, wherein the protease is selected from the group consisting of chymosin, trypsin, pepsin, matrix metalloproteinases, GI proteases, subtilisin, kex2 endoprotease, and TEV protease.

42. The method of claim 31, wherein the method further comprises cleaving the one or more linker peptides comprising the recognition or cleavage sequence for a protease with the protease, thereby releasing the casein.

43. The method of claim 31, wherein the recombinant host cell is derived from a bacterium.

44. The method of claim 31, wherein the recombinant host cell is derived from a fungus.

45. The method of claim 31, wherein the fungus is a yeast.

46. The method of claim 31, wherein the fungus is a filamentous fungus.

47. The method of claim 31, wherein the recombinant host cell is derived from a plant.

48. The method of claim 47, wherein the plant is soybean.

49. (canceled)

50. (canceled)

51. (canceled)

52. (canceled)

53. (canceled)

54. (canceled)

55. A composition comprising a recombinant protein having a structure:
(A) (milk protein 1)-(linker peptide 1) 6-(milk protein 2),
or
(B) (milk protein 1)-(linker peptide 1)-(milk protein 2)-(linker peptide 2)-(milk protein 3),
or
(C) (milk protein 1)-(linker peptide 1)-(milk protein 2)-(linker peptide 2)-(milk protein 3)-(linker peptide 3)-(milk protein 4);
wherein:
(i) the milk protein 1, the milk protein 2, the milk protein 3, the milk protein 4, the linker peptide 1, the linker peptide 2, and the linker peptide 3 are linked via peptide bonds; and
(ii) at least one of the milk protein 1, the milk protein 2, the milk protein 3, and the milk protein 4 is a milk protein comprising a free thiol group.

56. The composition of claim 55, wherein the composition is a food product.

57. The composition of claim 56, wherein the food product is a supplemented food product.

58. The composition of claim 57, wherein the supplemented food product is a supplemented dairy product.

59. The composition of claim 57, wherein the supplemented food product is a supplemented animal meat or animal meat product.

60. The composition of claim 57, wherein the supplemented food product is a supplemented egg or egg product.

61. The composition of claim 56, wherein the food product is a substitute food product.

62. The composition of claim 61, wherein the substitute food product is a substitute dairy product.

63. The composition of claim 61, wherein the substitute food product is a substitute animal meat or animal meat product.

64. The composition of claim 61, wherein the substitute food product is a substitute egg or egg product.

65. The composition of claim 55, wherein the composition is a cosmetic or personal care composition.

66. The composition of claim 56, wherein the composition is a film.

67. The composition of claim 56, wherein the composition is a coating.

68. The composition of claim 56, wherein the composition is a spray.

69. The composition of claim 56, wherein the composition is a gel, nanogel, hydrogel, hydronanogel, or hydrogel bead.

70. The composition of claim 56, wherein the composition is an encapsulate.

71. The composition of claim 56, wherein the composition is a microsphere, nanoparticle, nanofibril, or nanotube.

72. The composition of claim 56, wherein the composition is a nano fibril.

73. The composition of claim 56, wherein the composition is a hydro fibril.

74. The composition of claim 56, wherein the composition is a nanotube.

75. The composition of claim 56, wherein the composition is a foam.

76. The composition of claim 56, wherein the composition is a biological scaffold.

77. The composition of claim 56, wherein the composition is an encapsulate
